(19) United States
(12) Patent Application Publication
(10) Pub. No.: US 2025/0002868 A1
(43) Pub. Date: Jan. 2, 2025

(54) ADENO-ASSOCIATED VIRUS SEPARATION ON A CATION EXCHANGER

(71) Applicant: TAKEDA PHARMACEUTICAL COMPANY LIMITED, Osaka (JP)

(72) Inventors: Christian Fiedler, Vienna (AT); Dominik MITERGRADNEGGER, Vienna (AT); Meinhard HASSLACHER, Vienna (AT); Thomas GATTERNIG, Vienna (AT); Daniela VASINA, Vienna (AT); Michael FELDHOFER, Vienna (AT); Renate PFANDL, Bad Deutsch-Altenburg (AT)

(73) Assignee: TAKEDA PHARMACEUTICAL COMPANY LIMITED, Osaka (JP)

(21) Appl. No.: 18/294,941

(22) PCT Filed: Aug. 3, 2022
(86) PCT No.: PCT/IB2022/000436

§ 371 Date: Feb. 2, 2024

Related U.S. Application Data
(60) Provisional application No. 63/229,303, filed on Aug. 4, 2021.

(51) Int. Cl. C12N 7/00

(52) U.S. Cl. CPC C12N 7/00; C12N 2750/14151

(57) ABSTRACT

The present disclosure provides for a method of purifying adeno-associated virus comprising purifying full AAV capsids from a concentrated AAV fraction or preparation comprising empty AAV capsids and full AAV capsids. The present disclosure also provides for a method of purifying adeno-associated virus comprising purifying empty AAV capsids from a concentrated AAV fraction or preparation comprising empty AAV capsids and full AAV capsids. The method utilizes one or more monovalent and one or more divalent cations to effect the separation resulting in purified full AAV capsids or empty AAV capsids.

### CROSS-REFERENCE TO RELATED APPLICATIONS

The present application claims priority to U.S. Provisional Application No. 63/229,303, filed Aug. 4, 2021, which is hereby incorporated by reference in its entirety.

### BACKGROUND

Adeno-associated virus (AAV) is a small, non-enveloped virus that packages a linear single-stranded DNA genome. AAV belongs to the family Parvoviridae and the genus Dependovirus, since productive infection by AAV occurs only in the presence of a helper virus, such as, for example, adenovirus or herpes virus. Even in the absence of a helper virus, AAV (serotype 2) can achieve latency by integrating into chromosome 19q13.4 of a host human genome. It is the only mammalian DNA virus known to be capable of site-specific integration (Daya and Berns, Clinical Microbiology Reviews, pages 583-593 (2008)).

For AAV to be safely used in the clinic, AAV has been genetically modified at several locations within its genome. For example, the Rep gene, which is required for viral replication, and the element required for site-specific integration have been eliminated from the AAV genome in many viral vectors. These recombinant AAV (rAAV), exists in an extrachromosomal state and have very low integration efficiency into the genomic DNA. The possibility of rAAV inducing random mutagenesis in a host cell is thus reduced, if not eliminated altogether. Because of these properties and the lack of pathogenicity, rAAV has shown great promise as a gene therapy vector in multiple aspects of pre-clinical and clinical applications. New serotypes and self-complementary vectors are being tested in the clinic. Alongside these ongoing vector developments, continued effort has focused on scalable manufacturing processes that can efficiently generate high titer quantities of rAAV vectors with high purity and potency.

Though the effort to design efficient, large-scale methods to purify an AAV product suitable for human administration has been great, there still remains a need for better AAV purification methods. For example, current methods of generating AAV in cell culture result in the formation of “empty” capsids which have been shown to lead to T-cell-mediated immune responses against capsid antigen, leading to low-grade hepatotoxicity and partial loss of expression (Wright, Molec Therapy 22(1): 1-2 (2014)). AAV purification methods which include steps for removing empty AAV capsids from the final AAV product are therefore desired.

### SUMMARY OF THE INVENTION

A feature of AAV vector generation in cell culture is the formation of an excess of “empty” capsids, which lack the vector genome. Such empty capsids are unable to provide a therapeutic benefit associated with transgene production. The effect of the empty capsids on clinical outcome is not clear. However, there is a potential for increasing innate or adaptive immune responses to the vector, which then renders empty capsids a concern in gene therapy contexts. Wright, Molec Therapy 22(1): 1-2 (2014).

Provided herein is a method for purifying full AAV capsids from an AAV preparation comprising full AAV capsids and empty AAV capsids, to provide an AAV product substantially free of empty AAV capsids comprising the steps of:
(a) providing a first solution comprising empty AAV capsids, full AAV capsids, one or more monovalent cations, and one or more divalent cations;
(b) loading said first solution onto a cation exchange column under conditions whereby said full AAV capsids and said empty AAV capsids bind to the column; and
(c) adding a second solution comprising one or more monovalent cations and one or more divalent cations to the cation exchange column under conditions wherein the full AAV capsids are purified from the empty AAV capsids.

Provided herein is a method of separating full AAV capsids and empty AAV capsids in an AAV preparation comprising the steps of:
(a) providing a first solution comprising empty AAV capsids, full AAV capsids, one or more monovalent cations, and one or more divalent cations;
(b) loading said first solution onto a cation exchange column under conditions whereby said full AAV capsids and said empty AAV capsids bind to the column; and
(c) adding a second solution comprising one or more monovalent cations and one or more divalent cations to the cation exchange column under conditions wherein the full AAV capsids are separated from the empty AAV capsids.

Provided herein is a method of separating empty AAV capsids and full AAV capsids in an AAV preparation comprising the steps of:
(a) providing a first solution comprising empty AAV capsids, full AAV capsids, one or more monovalent cations, and one or more divalent cations;
(b) loading said first solution onto a cation exchange column under conditions whereby said empty AAV capsids and said full AAV capsids bind to the column; and
(c) adding a second solution comprising one or more monovalent cations and one or more divalent cations to the cation exchange column under conditions wherein the empty AAV capsids are purified from the full AAV capsids.

Provided herein is a method of separating empty AAV capsids and full AAV capsids in an AAV preparation comprising the steps of:
(a) providing a first solution comprising empty AAV capsids, full AAV capsids, one or more monovalent cations, and one or more divalent cations;
(b) loading said first solution onto a cation exchange column under conditions whereby said empty AAV capsids and said full AAV capsids bind to the column; and
(c) adding a second solution comprising one or more monovalent cations and one or more divalent cations to the cation exchange column under conditions wherein the empty AAV capsids are separated from the full AAV capsids

In some aspects, the disclosed method is also useful for purifying AdV particles, lentiviral particles, gammaretroviral vector particles, herpes simples virus (HSV) particles, simian virus 40 (SV40) particles, alphavirus particle, togavirdae particles, Ross river virus particles, and Vaccinia virus particles. In some aspects, the disclosed method is also useful for producing vector vaccines.

In some embodiments, the one or more monovalent cations of the first solution is selected from the group consisting of Na⁺, K⁺, NH₄⁺, Li⁺, Cs⁺, and combinations thereof. In some embodiments, the one or more monovalent cation of the first solution is Na⁺.

In some embodiments, the one or more monovalent cations of the first solution is in a total concentration of about 5 mM to about 1500 mM. In some embodiments, the one or more monovalent cations of the first solution is in a total concentration of about 30 mM.

In some embodiments, the one or more divalent cations of the first solution is selected from the group consisting of Ca²⁺, Mg²⁺, Zn²⁺, Mn²⁺, Cu²⁺, Fe²⁺, Ba²⁺, Sr²⁺, Co²⁺, and combinations thereof. In some embodiments, the one or more divalent cation of the first solution is Ca²⁺.

In some embodiments, the one or more divalent cations of the first solution is in a total concentration of about 1 mM to about 30 mM. In some embodiments, the one or more divalent cations of the first solution is in a total concentration of about 2 mM.

In some embodiments, the first solution has a pH of about 5.0 to about 8.5. In some embodiments, the first solution has a pH of about 6.0.

In some embodiments, the first solution further comprises one or more surfactants. In some embodiments, the one or more surfactants is selected from the group consisting of polysorbate 20, polysorbate 40, polysorbate 65, polysorbate 80, polyoxyethylene glycol tert-octylphenol ether, sorbitan monolaurate, sorbitan monopalmitate, sorbitan monostearate, sorbitan tristearate, sorbitan monooleate, sorbitan trioleate, polyoxyethylene (20) sorbitan monopalmitate, polyoxyethylene (20) sorbitan monostearate, polyoxyethylene (20) sorbitan tristearate, polyoxyethylene (20) sorbitan trioleate, polyoxyethylen(20)-sorbitan-monooleate (Tween 80/Polysorbate 80)), poloxamer 124, poloxamer 188, poloxamer 407, cremophor, Triton N-101 reduced, Triton X-100, and combinations thereof. In some embodiments, surfactant is polysorbate 80.

In some embodiments, the one or more surfactants is in a total amount of about 0.0025 w/w % to about 0.0075 w/w %. In some embodiments, the one or more surfactants is in a total amount of about 0.005 w/w %.

In some embodiments, the cation exchange column comprises a resin with a charged group wherein the charged group is sulfonate, sulfate, sulfopropyl, carboxyl, phosphate, or combinations thereof. In some embodiments, the cation exchange column comprises a resin wherein it the resin is Capto S, Eshmuno S, Mustang S, Poros 50HS, Poros 50 XS, S-Sepharose FF, Source S, Capto MMC, Toyopearl Gigacap S, Gigacap CM, Toyopearl SP, Toyopearl CM, MacroPrep S, UNOsphereS, MacroprepCM, Fractogel EMD SO3, Fractogel EMD COO, Fractogel EMD SE Hicap, Cellufine Sulfate, CM and SP Trisacryl, CM and S HyperD, S and CM Sepharose CL, CM Sepharose FF, S and CM CAPTO™, MonoS, Nuvia S, Cellufine phosphat, Cellufine MAX-S r, Cellufine MAX-S h, Cellufine MAX DexS-HbP, Cellufine MAX DexS-VirS, Toyopearl Sulfate 650, or Heparin Sepharose High Performance. In some embodiments, CaptoS. In some embodiments, Eshmuno S. In some embodiments, Mustang S.

In some embodiments, the one or more monovalent cations of the second solution is selected from the group consisting of Na⁺, K⁺, NH₄⁺, Li⁺, Cs⁺, and combinations thereof. In some embodiments, the monovalent cation of the second solution is Na⁺.

In some embodiments, the one or more divalent cations of the second solution is selected from the group consisting of Ca²⁺, Mg²⁺, Zn²⁺, Mn²⁺, Cu²⁺, Fe²⁺, Ba²⁺, Sr²⁺, and combinations thereof. In some embodiments, the one or more divalent cation of the second solution is Ca²⁺.

In some embodiments, the second solution has a pH of about 5.0 to about 8.5. In some embodiments, the second solution has a pH of about 6.0.

In some embodiments, the second solution further comprises one or more surfactants. In some embodiments, the one or more surfactants is selected from the group consisting of polysorbate 20, polysorbate 40, polysorbate 65, polysorbate 80, polyoxyethylene glycol tert-octylphenol ether, sorbitan monolaurate, sorbitan monopalmitate, sorbitan monostearate, sorbitan tristearate, sorbitan monooleate, sorbitan trioleate, polyoxyethylene (20) sorbitan monopalmitate, polyoxyethylene (20) sorbitan monostearate, polyoxyethylene (20) sorbitan tristearate, polyoxyethylene (20) sorbitan trioleate, polyoxyethylen(20)-sorbitan-monooleate (Tween 80/Polysorbate 80)), poloxamer 124, poloxamer 188, poloxamer 407, cremophor, Triton N-101 reduced, Triton X-100,and combinations thereof. In some embodiments, the surfactant is polysorbate 80.

In some embodiments, the one or more surfactants is in a total amount of about 0.00 w/w25% to about 0.0075 w/w %. In some embodiments, the one or more surfactants is in a total amount of about 0.005 w/w %.

In some aspects, adding the second solution is carried out at a constant concentration of the one or more monovalent cations.

In some embodiments, the one or more monovalent cations of the second solution is in a constant total concentration of about 5 mM to about 1500 mM. In some embodiments, the one or more monovalent cations of the second solution is in a constant total concentration of about 30 mM.

In some aspects, adding the second solution is carried out at a constant concentration of the one or more divalent cations.

In some embodiments, the one or more divalent cations of the second solution is in a constant total concentration of about 1 mM to about 30 mM. In some embodiments, the one or more divalent cations of the second solution is in a constant total concentration of about 2 mM.

In some aspects, adding the second solution comprises a stepwise increase of the concentration of the one or more monovalent cations.

In some embodiments, the initial total concentration of the one or more monovalent cations of the second solution is about 15 mM to about 60 mM. In some embodiments, the initial total concentration of the one or more monovalent cations of the second solution is about 30 mM. In some embodiments, the intermediate total concentration of the one or more monovalent cations of the second solution is about 100 mM to about 300 mM. In some embodiments, the intermediate total concentration of the one or more monovalent cations of the second solution is about 200 mM. In some embodiments, the final total concentration of the one or more monovalent cations of the second solution is about 500 mM to about 1500 mM. In some embodiments, the final total concentration of the one or more monovalent cations of the second solution is about 1000 mM.

In some aspects, adding the second solution comprises a stepwise increase of the total concentration of the one or more divalent cations.

In certain embodiments, the initial total concentration of the one or more divalent cations of the second solution is about 1 mM to about 10 mM. In certain embodiments, the intermediate total concentration of the one or more divalent cations of the second solution is about 10 mM to about 20 mM. In certain embodiments, the final total concentration of the one or more divalent cations of the second solution is about 20 mM to about 30 mM.

In some aspects, adding the second solution comprises a continuous linear increase of the total concentration of the one or more monovalent cations.

In certain embodiments, the continuous linear increase of the total concentration of the one or more monovalent cations of the second solution is about 30 mM to about 200 mM in 40 column volumes. In certain embodiments, the continuous linear increase of the total concentration of the one or more monovalent cations of the second solution is about 30 mM to about 80 mM in 40 column volumes. In certain embodiments, the continuous linear increase of the total concentration of the one or more monovalent cations of the second solution is about 30 mM to about 40 mM in 40 column volumes. In certain embodiments, the continuous linear increase of the total concentration of the one or more monovalent cations of the second solution is about 30 mM to about 200 mM in 5 column volumes. In certain embodiments, the continuous linear increase of the total concentration of the one or more monovalent cations of the second solution is about 30 mM to about 80 mM in 5 column volumes. In certain embodiments, the continuous linear increase of the total concentration of the one or more monovalent cations of the second solution is about 30 mM to about 80 mM in 5 column volumes.

In some aspects, adding the second solution comprises a continuous linear increase of the total concentration of the one or more divalent cations.

In certain embodiments, the continuous linear increase of the total concentration of the one or more divalent cations of the second solution is about 1 mM to about 30 mM in 40 column volumes. In certain embodiments, the continuous linear increase of the total concentration of the one or more divalent cations of the second solution is about 1 mM to about 15 mM in 40 column volumes. In certain embodiments, the continuous linear increase of the total concentration of the one or more divalent cations of the second solution is about 1 mM to about 5 mM in 40 column volumes. In certain embodiments, the continuous linear increase of the total concentration of the one or more divalent cations of the second solution is about 1 mM to about 30 mM in 5 column volumes. In certain embodiments, the continuous linear increase of the total concentration of the one or more divalent cations of the second solution is about 1 mM to about 15 mM in 5 column volumes. In certain embodiments, the continuous linear increase of the total concentration of the one or more divalent cations of the second solution is about 1 mM to about 5 mM in 5 column volumes.

In certain embodiments, the AAV capsid is derived from the group consisting of AAV2, AAV3, AAV3b, AAV4, AAV5, AAV6, AAV7, AAV8, AAV9, AAV10, AAV11, AAV12, genetical modified AAV, chemical modified AAV, genetical and chemical modified AAV, and combinations thereof. In certain embodiments, the AAV capsid is derived from AAV8. In certain embodiments, the AAV capsid is derived from AAV9. In certain embodiments, the AAV capsid is derived from AAV6.

In certain aspects, the methods further comprise preparing an immune absorption column comprising the steps of
(a) concentrating the empty AAV capsids by ultrafiltration, anion exchanger, and/or cation exchanger;
(b) applying a buffer exchange into an amine free buffer; and
(c) immobilizing the empty AAV capsids on an activated resin.

Also provided herein is an AAV formulation comprising full AAV capsids purified according to the method as described herein. In certain embodiments, the AAV formulation further comprises a pharmaceutically acceptable carrier. In certain embodiments, the AAV formulation is substantially free of empty AAV capsids.

Also provided herein is a pharmaceutical composition comprising an AAV product, formulation, or composition produced by a method as described herein. In certain embodiments, the AAV pharmaceutical composition further comprises a pharmaceutically acceptable carrier. In certain embodiments, the AAV pharmaceutical composition is substantially free of empty AAV capsids.

### BRIEF DESCRIPTION OF THE DRAWINGS

FIG. 1 depicts the complete chromatogram of Example 2.

FIG. 2 depicts the elution zone chromatograph of Example 2.

FIG. 3 depicts the area under curve (AUC) profile for fraction E2.

FIG. 4 depicts the AUC profile for fraction E3.

FIG. 5 depicts the AUC profile for fraction E4.

FIG. 6 depicts the AUC profile for fraction E5.

FIG. 7 depicts the complete chromatogram of Example 3.

FIG. 8 depicts the elution zone chromatograph of Example 3.

FIG. 9 depicts the AUC profile for fraction E2.

FIG. 10 depicts the AUC profile for fraction E3.

FIG. 11 depicts the AUC profile for fraction E5.

FIG. 12 depicts the complete chromatogram of Example 4.

FIG. 13 depicts the elution zone chromatograph of Example 4.

FIG. 14 depicts AUC profile for fraction E2.

FIG. 15 depicts the AUC profile for fraction E3.

FIG. 16 depicts the AUC profile for fraction E4.

FIG. 17 depicts the AUC profile for fraction E5.

FIG. 18 depicts the complete chromatogram of Example 5.

FIG. 19 depicts the elution zone chromatograph of Example 5.

FIG. 20 depicts AUC profile for fraction E1.

FIG. 21 depicts the AUC profile for fraction E2.

FIG. 22 depicts the AUC profile for fraction E3.

FIG. 23 depicts the AUC profile for fraction E4.

FIG. 24 depicts the AUC profile for fraction E5.

FIG. 25 depicts the complete chromatogram of Example 6.

FIG. 26 depicts the elution zone chromatograph of Example 6.

FIG. 27 depicts AUC profile for fraction E1.

FIG. 28 depicts the AUC profile for fraction E2.

FIG. 29 depicts the AUC profile for fraction E3.

FIG. 30 depicts the AUC profile for fraction E4.

FIG. 31 depicts the AUC profile for fraction E5.

FIG. 32 depicts the AUC profile for fraction E6.

FIG. 33 depicts the elution zone chromatograph of Example 7.

FIG. 34 depicts AUC profile for fraction E1.

FIG. 35 depicts the AUC profile for fraction E2.

FIG. 36 depicts the AUC profile for fraction E3.

FIG. 37 depicts the AUC profile for fraction E4.

FIG. 38 depicts the AUC profile for fraction E5.

### DEFINITIONS

Before the present compositions and methods are described, it is to be understood that this invention is not limited to the particular processes, compositions, or methodologies described, as these may vary. It is also to be understood that the terminology used in the description is for the purpose of describing the particular versions or embodiments only and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

As used herein, the terms below have the meanings indicated.

The use of the terms “a,” “an” and “the”, and similar referents in the context of describing the disclosure (especially in the context of the following claims), are to be construed to cover both the singular and the plural, unless otherwise indicated herein or clearly contradicted by context.

In some embodiments, if aspects of the disclosure are described as “comprising”, or versions thereof (e.g., comprises), a feature, embodiments also are contemplated “consisting of” or “consisting essentially of” the feature

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean plus or minus 10% of the numerical value of the number with which it is being used. Therefore, about 50% means in the range of 45%-55%.

The term “alkyl,” as used herein, alone or in combination, refers to a straight-chain or branched-chain alkyl radical. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, nonyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

As used herein, the terms “capsid”, “capsid particle”, and “particle” are used interchangeably and refer to an AAV particle composed of at least one intact AAV capsid shell.

As used herein, the term “empty” with regard to AAV or AAV capsids or AAV particles refers to those that lack the complete (i.e., full) vector genome. Empty AAV or empty AAV capsids or empty AAV particles are unable to provide a therapeutic benefit. As used herein, the term “full” or “full AAV capsids” with regard to AAV or AAV capsids or AAV particles refer to those containing a majority of the complete vector genome. Full AAV capsids can provide a therapeutic benefit to recipient patients. In certain embodiments, “full” can also include “incomplete vector DNA” or “truncated vector DNA”. In certain embodiments, complete versus incomplete and/or truncated vector DNA can be differentiated with additional analytic methods. Such methods include, without limitation, DNA sizing by capillary electrophoresis, AUC (analytical ultracentrifugation), % Agarose DNA (native or alkaline), gel, southern blot, dot-blot hybridization, UV spectrophotometry, weak anion exchange chromatography, and mass spectrometry (See Resolving Adeno-Associated Viral Particle Diversity with Charge Detection Mass Spectrometry Elizabeth E. Pierson et. al Anal. Chem., 2016, 88 (13), pp 6718-6725, which is incorporated herein in its entirety for all purposes).

The terms “patient” and “subject” are used interchangeably and are used in their conventional sense to refer to a living organism suffering from or prone to a condition that can be prevented or treated by administration of an AAV product, formulation, or composition of the present disclosure, and includes both humans and non-human animals. Examples of subjects include, but are not limited to, humans, chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, adult, juvenile and newborn individuals are of interest.

An AAV product, an AAV formulation, or any AAV containing composition is “substantially free” of empty AAV capsids when it comprises less than about 30% of empty AAV capsids.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. For example, it is specifically understood that any numerical value recited herein includes all values from the lower value to the upper value, i.e., all possible combinations of numerical values between the lowest value and the highest value enumerated are to be considered to be expressly stated in this application. For example, if a concentration range is stated as about 1% to 50%, it is intended that values such as 2% to 40%, 10% to 30%, or 1% to 3%, etc., are expressly enumerated in this specification. The values listed above are only examples of what is specifically intended.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

### DETAILED DESCRIPTION

Provided herein are methods of producing an adeno-associated virus (AAV) product, formulation, or composition, methods of purifying AAV, methods of purifying full AAV capsids from an AAV preparation or fraction comprising full AAV capsids and empty AAV capsids, methods of purifying empty AAV capsids from a AAV preparation or fraction comprising empty AAV capsids and full AAV capsids, and methods of preparing an immune absorption column with both purified empty AAV capsids and purified full AAV capsids.

In certain embodiments, the method for purifying full AAV capsids from an AAV preparation or fraction comprising full AAV capsids and empty AAV capsids, to provide an AAV product, formulation, or composition substantially free of empty AAV capsids comprising the steps of
(a) providing a first solution comprising full AAV capsids, empty AAV capsids, one or more monovalent cations, and one or more divalent cations;
(b) loading said first solution onto a cation exchange column under conditions whereby said full AAV capsids and said empty AAV capsids bind to the column; and
(c) adding a second solution comprising one or more monovalent cations and one or more divalent cations to the cation exchange column under conditions wherein the full AAV capsids are purified from the empty AAV capsids.

In certain embodiments, the method of separating full AAV capsids and empty AAV capsids in an AAV preparation or fraction comprising the steps of:
(a) providing a first solution comprising full AAV capsids, empty AAV capsids, one or more monovalent cations, and one or more divalent cations;
(b) loading said first solution onto a cation exchange column under conditions whereby said full AAV capsids and said empty AAV capsids bind to the column; and
(c) adding a second solution comprising one or more monovalent cations and one or more divalent cations to the cation exchange column under conditions wherein the full AAV capsids are separated from the empty AAV capsids.

In some embodiments, the one or more monovalent cations of the first solution is selected from the group consisting of Na⁺, K⁺, NH₄⁺, N(C₁₋₅alkyl)₄⁺, Li⁺, Cs⁺, Cu⁺, Ag⁺, Au⁺, and combinations thereof.

In some embodiments, the one or more monovalent cations of the first solution is selected from the group consisting of Na⁺, K⁺, NH₄⁺, Li⁺, Cs⁺, and combinations thereof.

In some embodiments, the one or more monovalent cations of the first solution is selected from the group consisting of Na⁺, K⁺, NH₄⁺, and combinations thereof.

In some embodiments, the one or more monovalent cations of the first solution is Na⁺.

In some embodiments, the one or more monovalent cations of the first solution is K⁺.

In some embodiments, the one or more monovalent cations of the first solution is NH₄⁺.

In some embodiments, the one or more monovalent cations of the first solution is in a total concentration of about 5 mM to about 1500 mM, about 50 mM to about 1500 mM, about 100 mM to about 1500 mM, about 200 mM to about 1500 mM, about 300 mM to about 1500 mM, about 400 mM to about 1500 mM, about 500 mM to about 1500 mM, about 600 mM to about 1500 mM, about 700 mM to about 1500 mM, about 800 mM to about 1500 mM, about 900 mM to about 1500 mM, about 1000 mM to about 1500 mM, about 1100 mM to about 1500 mM, about 1200 mM to about 1500 mM, about 1300 mM to about 1500 mM, about 1400 mM to about 1500 mM, about 5 mM to about 1400 mM, about 5 mM to about 1300 mM, about 5 mM to about 1200 mM, about 5 mM to about 1100 mM, about 5 mM to about 1000 mM, about 5 mM to about 900 mM, about 5 mM to about 800 mM, about 5 mM to about 700 mM, about 5 mM to about 600 mM, about 5 mM to about 500 mM, about 5 mM to about 400 mM, about 5 mM to about 300 mM, about 5 mM to about 200 mM, about 5 mM to about 100 mM, about 5 mM to about 50 mM, 30 mM to about 200 mM, 30 mM to about 80 mM, 30 mM to about 60 mM, or a value within one of these ranges. Specific examples may include about 5 mM, about 10 mM, about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, about 600 mM, about 700 mM, about 800 mM, about 900 mM, about 1000 mM, about 1100 mM, about 1200 mM, about 1300 mM, about 1400 mM, about 1500 mM, or a range between any two of these values.

In some embodiments, the one or more monovalent cations of the first solution is in a total concentration of about 5 mM to about 1500 mM.

In some embodiments, the one or more monovalent cations of the first solution is in a total concentration of about 30 mM to about 200 mM.

In some embodiments, the one or more monovalent cations of the first solution is in a total concentration of about 30 mM to about 80 mM.

In some embodiments, the one or more monovalent cations of the first solution is in a total concentration of about 30 mM to about 60 mM.

In some embodiments, the one or more monovalent cations of the first solution is in a total concentration of about 200 mM.

In some embodiments, the one or more monovalent cations of the first solution is in a total concentration of about 80 mM.

In some embodiments, the one or more monovalent cations of the first solution is in a total concentration of about 60 mM.

In some embodiments, the one or more monovalent cations of the first solution is in a total concentration of about 30 mM.

In some embodiments, the one or more monovalent cations of the first solution is Na⁺ and is in a total concentration of about 5 mM to about 1500 mM, about 50 mM to about 1500 mM, about 100 mM to about 1500 mM, about 200 mM to about 1500 mM, about 300 mM to about 1500 mM, about 400 mM to about 1500 mM, about 500 mM to about 1500 mM, about 600 mM to about 1500 mM, about 700 mM to about 1500 mM, about 800 mM to about 1500 mM, about 900 mM to about 1500 mM, about 1000 mM to about 1500 mM, about 1100 mM to about 1500 mM, about 1200 mM to about 1500 mM, about 1300 mM to about 1500 mM, about 1400 mM to about 1500 mM, about 5 mM to about 1400 mM, about 5 mM to about 1300 mM, about 5 mM to about 1200 mM, about 5 mM to about 1100 mM, about 5 mM to about 1000 mM, about 5 mM to about 900 mM, about 5 mM to about 800 mM, about 5 mM to about 700 mM, about 5 mM to about 600 mM, about 5 mM to about 500 mM, about 5 mM to about 400 mM, about 5 mM to about 300 mM, about 5 mM to about 200 mM, about 5 mM to about 100 mM, about 5 mM to about 50 mM, 30 mM to about 200 mM, 30 mM to about 80 mM, 30 mM to about 60 mM, or a value within one of these ranges. Specific examples may include about 5 mM, about 10 mM, about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, about 600 mM, about 700 mM, about 800 mM, about 900 mM, about 1000 mM, about 1100 mM, about 1200 mM, about 1300 mM, about 1400 mM, about 1500 mM, or a range between any two of these values.

In some embodiments, the one or more monovalent cations of the first solution is Na⁺ and is in a total concentration of about 5 mM to about 1500 mM.

In some embodiments, the one or more monovalent cations of the first solution is Na⁺ and is in a total concentration of about 30 mM to about 200 mM.

In some embodiments, the one or more monovalent cations of the first solution is Na⁺ and is in a total concentration of about 30 mM to about 80 mM.

In some embodiments, the one or more monovalent cations of the first solution is Na⁺ and is in a total concentration of about 30 mM to about 60 mM.

In some embodiments, the one or more monovalent cations of the first solution is Na⁺ and is in a total concentration of about 200 mM.

In some embodiments, the one or more monovalent cations of the first solution is Na⁺ and is in a total concentration of about 80 mM.

In some embodiments, the one or more monovalent cations of the first solution is Na⁺ and is in a total concentration of about 60 mM.

In some embodiments, the one or more monovalent cations of the first solution is Na⁺ and is in a total concentration of about 30 mM.

In some embodiments, the one or more monovalent cations of the first solution is K⁺ and is in a total concentration of about 5 mM to about 1500 mM, about 50 mM to about 1500 mM, about 100 mM to about 1500 mM, about 200 mM to about 1500 mM, about 300 mM to about 1500 mM, about 400 mM to about 1500 mM, about 500 mM to about 1500 mM, about 600 mM to about 1500 mM, about 700 mM to about 1500 mM, about 800 mM to about 1500 mM, about 900 mM to about 1500 mM, about 1000 mM to about 1500 mM, about 1100 mM to about 1500 mM, about 1200 mM to about 1500 mM, about 1300 mM to about 1500 mM, about 1400 mM to about 1500 mM, about 5 mM to about 1400 mM, about 5 mM to about 1300 mM, about 5 mM to about 1200 mM, about 5 mM to about 1100 mM, about 5 mM to about 1000 mM, about 5 mM to about 900 mM, about 5 mM to about 800 mM, about 5 mM to about 700 mM, about 5 mM to about 600 mM, about 5 mM to about 500 mM, about 5 mM to about 400 mM, about 5 mM to about 300 mM, about 5 mM to about 200 mM, about 5 mM to about 100 mM, about 5 mM to about 50 mM, 30 mM to about 200 mM, 30 mM to about 80 mM, 30 mM to about 60 mM, or a value within one of these ranges. Specific examples may include about 5 mM, about 10 mM, about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, about 600 mM, about 700 mM, about 800 mM, about 900 mM, about 1000 mM, about 1100 mM, about 1200 mM, about 1300 mM, about 1400 mM, about 1500 mM, or a range between any two of these values.

In some embodiments, the one or more monovalent cations of the first solution is K⁺ and is in a total concentration of about 5 mM to about 1500 mM.

In some embodiments, the one or more monovalent cations of the first solution is K⁺ and is in a total concentration of about 30 mM to about 200 mM.

In some embodiments, the one or more monovalent cations of the first solution is K⁺ and is in a total concentration of about 30 mM to about 80 mM.

In some embodiments, the one or more monovalent cations of the first solution is K⁺ and is in a total concentration of about 30 mM to about 60 mM.

In some embodiments, the one or more monovalent cations of the first solution is K⁺ and is in a total concentration of about 200 mM.

In some embodiments, the one or more monovalent cations of the first solution is K⁺ and is in a total concentration of about 80 mM.

In some embodiments, the one or more monovalent cations of the first solution is K⁺ and is in a total concentration of about 60 mM.

In some embodiments, the one or more monovalent cations of the first solution is K⁺ and is in a total concentration of about 30 mM.

In some embodiments, the one or more monovalent cations of the first solution is NH₄⁺ and is in a total concentration of about 5 mM to about 1500 mM, about 50 mM to about 1500 mM, about 100 mM to about 1500 mM, about 200 mM to about 1500 mM, about 300 mM to about 1500 mM, about 400 mM to about 1500 mM, about 500 mM to about 1500 mM, about 600 mM to about 1500 mM, about 700 mM to about 1500 mM, about 800 mM to about 1500 mM, about 900 mM to about 1500 mM, about 1000 mM to about 1500 mM, about 1100 mM to about 1500 mM, about 1200 mM to about 1500 mM, about 1300 mM to about 1500 mM, about 1400 mM to about 1500 mM, about 5 mM to about 1400 mM, about 5 mM to about 1300 mM, about 5 mM to about 1200 mM, about 5 mM to about 1100 mM, about 5 mM to about 1000 mM, about 5 mM to about 900 mM, about 5 mM to about 800 mM, about 5 mM to about 700 mM, about 5 mM to about 600 mM, about 5 mM to about 500 mM, about 5 mM to about 400 mM, about 5 mM to about 300 mM, about 5 mM to about 200 mM, about 5 mM to about 100 mM, about 5 mM to about 50 mM, 30 mM to about 200 mM, 30 mM to about 80 mM, 30 mM to about 60 mM, or a value within one of these ranges. Specific examples may include about 5 mM, about 10 mM, about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, about 600 mM, about 700 mM, about 800 mM, about 900 mM, about 1000 mM, about 1100 mM, about 1200 mM, about 1300 mM, about 1400 mM, about 1500 mM, or a range between any two of these values.

In some embodiments, the one or more monovalent cations of the first solution is NH₄⁺ and is in a total concentration of about 5 mM to about 1500 mM.

In some embodiments, the one or more monovalent cations of the first solution is NH₄⁺ and is in a total concentration of about 30 mM to about 200 mM.

In some embodiments, the one or more monovalent cations of the first solution is NH₄⁺ and is in a total concentration of about 30 mM to about 80 mM.

In some embodiments, the one or more monovalent cations of the first solution is NH₄⁺ and is in a total concentration of about 30 mM to about 60 mM.

In some embodiments, the one or more monovalent cations of the first solution is NH₄⁺ and is in a total concentration of about 200 mM.

In some embodiments, the one or more monovalent cations of the first solution is NH₄⁺ and is in a total concentration of about 80 mM.

In some embodiments, the one or more monovalent cations of the first solution is NH₄⁺ and is in a total concentration of about 60 mM.

In some embodiments, the one or more monovalent cations of the first solution is NH₄⁺ and is in a total concentration of about 30 mM.

In some embodiments, the one or more divalent cations of the first solution is selected from the group consisting of Ca²⁺, Mg²⁺, Zn²⁺, Mn²⁺, Cu²⁺, Fe²⁺, Ba²⁺, Sr²⁺, Co²⁺, Be²⁺, Ga²⁺, Pb²⁺, Sr²⁺, Ti²⁺, Sr²⁺, and combinations thereof.

In some embodiments, the one or more divalent cations of the first solution is selected from the group consisting of Ca²⁺, Mg²⁺, Zn²⁺, Mn²⁺, Cu²⁺, Fe²⁺, Ba²⁺, Sr²⁺, Co²⁺, and combinations thereof.

In some embodiments, the one or more divalent cations of the first solution is selected from the group consisting of Ca²⁺, Mg²⁺, Zn²⁺, Mn²⁺, Cu²⁺, and combinations thereof.

In some embodiments, the one or more divalent cations of the first solution is Ca²⁺.

In some embodiments, the one or more divalent cations of the first solution is Mg²⁺.

In some embodiments, the one or more divalent cations of the first solution is in a total concentration of about 1 mM to about 30 mM, about 5 mM to about 30 mM, about 10 mM to about 30 mM, about 15 mM to about 30 mM, about 20 mM to about 30 mM, about 25 mM to about 30 mM, about 1 mM to about 25 mM, about 1 mM to about 20 mM, about 1 mM to about 15 mM, about 1 mM to about 10 mM, about 1 mM to about 5 mM, or a value within one of these ranges. Specific examples may include about 1 mM, about 2 mM, about 3 mM, about 4 mM, about 5 mM, about 6 mM, about 7 mM, about 8 mM, about 9 mM, 10 mM, about 11 mM, about 12 mM, about 13 mM, about 14 mM, about 15 mM, about 16 mM, about 17 mM, about 18 mM, about 19 mM, 20 mM, about 21 mM, about 22 mM, about 23 mM, about 24 mM, about 25 mM, about 26 mM, about 27 mM, about 28 mM, about 29 mM, about 30 mM, or a range between any two of these values.

In some embodiments, the one or more divalent cations of the first solution is in a total concentration of about 1 mM to about 30 mM.

In some embodiments, the one or more divalent cations of the first solution is in a total concentration of about 1 mM to about 10 mM.

In some embodiments, the one or more divalent cations of the first solution is in a total concentration of about 2 mM.

In some embodiments, the one or more divalent cations of the first solution is Ca²⁺ and is in a total concentration of about 1 mM to about 30 mM, about 5 mM to about 30 mM, about 10 mM to about 30 mM, about 15 mM to about 30 mM, about 20 mM to about 30 mM, about 25 mM to about 30 mM, about 1 mM to about 25 mM, about 1 mM to about 20 mM, about 1 mM to about 15 mM, about 1 mM to about 10 mM, about 1 mM to about 5 mM, or a value within one of these ranges. Specific examples may include about 1 mM, about 2 mM, about 3 mM, about 4 mM, about 5 mM, about 6 mM, about 7 mM, about 8 mM, about 9 mM, 10 mM, about 11 mM, about 12 mM, about 13 mM, about 14 mM, about 15 mM, about 16 mM, about 17 mM, about 18 mM, about 19 mM, 20 mM, about 21 mM, about 22 mM, about 23 mM, about 24 mM, about 25 mM, about 26 mM, about 27 mM, about 28 mM, about 29 mM, about 30 mM, or a range between any two of these values.

In some embodiments, the one or more divalent cations of the first solution is Ca²⁺ and is in a total concentration of about 1 mM to about 30 mM.

In some embodiments, the one or more divalent cations of the first solution is Ca²⁺ and is in a total concentration of about 1 mM to about 10 mM.

In some embodiments, the one or more divalent cations of the first solution is Ca²⁺ and is in a total concentration of about 2 mM.

In some embodiments, the one or more divalent cations of the first solution is Mg²⁺ and is in a total concentration of about 1 mM to about 30 mM, about 5 mM to about 30 mM, about 10 mM to about 30 mM, about 15 mM to about 30 mM, about 20 mM to about 30 mM, about 25 mM to about 30 mM, about 1 mM to about 25 mM, about 1 mM to about 20 mM, about 1 mM to about 15 mM, about 1 mM to about 10 mM, about 1 mM to about 5 mM, or a value within one of these ranges. Specific examples may include about 1 mM, about 2 mM, about 3 mM, about 4 mM, about 5 mM, about 6 mM, about 7 mM, about 8 mM, about 9 mM, 10 mM, about 11 mM, about 12 mM, about 13 mM, about 14 mM, about 15 mM, about 16 mM, about 17 mM, about 18 mM, about 19 mM, 20 mM, about 21 mM, about 22 mM, about 23 mM, about 24 mM, about 25 mM, about 26 mM, about 27 mM, about 28 mM, about 29 mM, about 30 mM, or a range between any two of these values.

In some embodiments, the one or more divalent cations of the first solution is Mg²⁺ and is in a total concentration of about 1 mM to about 30 mM.

In some embodiments, the one or more divalent cations of the first solution is Mg²⁺ and is in a total concentration of about 1 mM to about 10 mM.

In some embodiments, the one or more divalent cations of the first solution is Mg²⁺ and is in a total concentration of about 2 mM.

In some embodiments, the one or more divalent cations of the first solution is Mg²⁺ and is in a total concentration of about 1 mM to about 30 mM, about 5 mM to about 30 mM, about 10 mM to about 30 mM, about 15 mM to about 30 mM, about 20 mM to about 30 mM, about 25 mM to about 30 mM, about 1 mM to about 25 mM, about 1 mM to about 20 mM, about 1 mM to about 15 mM, about 1 mM to about 10 mM, about 1 mM to about 5 mM, or a value within one of these ranges. Specific examples may include about 1 mM, about 2 mM, about 3 mM, about 4 mM, about 5 mM, about 6 mM, about 7 mM, about 8 mM, about 9 mM, 10 mM, about 11 mM, about 12 mM, about 13 mM, about 14 mM, about 15 mM, about 16 mM, about 17 mM, about 18 mM, about 19 mM, 20 mM, about 21 mM, about 22 mM, about 23 mM, about 24 mM, about 25 mM, about 26 mM, about 27 mM, about 28 mM, about 29 mM, about 30 mM, or a range between any two of these values.

In some embodiments, the one or more divalent cations of the first solution is Mg²⁺ and is in a total concentration of about 1 mM to about 30 mM.

In some embodiments, the one or more divalent cations of the first solution is Mg²⁺ and is in a total concentration of about 1 mM to about 10 mM.

In some embodiments, the one or more divalent cations of the first solution is Mg²⁺ and is in a total concentration of about 2 mM.

In some embodiments, the one or more divalent cations of the first solution is Zn²⁺ and is in a total concentration of about 1 mM to about 30 mM, about 5 mM to about 30 mM, about 10 mM to about 30 mM, about 15 mM to about 30 mM, about 20 mM to about 30 mM, about 25 mM to about 30 mM, about 1 mM to about 25 mM, about 1 mM to about 20 mM, about 1 mM to about 15 mM, about 1 mM to about 10 mM, about 1 mM to about 5 mM, or a value within one of these ranges. Specific examples may include about 1 mM, about 2 mM, about 3 mM, about 4 mM, about 5 mM, about 6 mM, about 7 mM, about 8 mM, about 9 mM, 10 mM, about 11 mM, about 12 mM, about 13 mM, about 14 mM, about 15 mM, about 16 mM, about 17 mM, about 18 mM, about 19 mM, 20 mM, about 21 mM, about 22 mM, about 23 mM, about 24 mM, about 25 mM, about 26 mM, about 27 mM, about 28 mM, about 29 mM, about 30 mM, or a range between any two of these values.

In some embodiments, the one or more divalent cations of the first solution is Zn²⁺ and is in a total concentration of about 1 mM to about 30 mM.

In some embodiments, the one or more divalent cations of the first solution is Zn²⁺ and is in a total concentration of about 1 mM to about 10 mM.

In some embodiments, the one or more divalent cations of the first solution is Zn²⁺ and is in a total concentration of about 2 mM.

In some embodiments, the one or more divalent cations of the first solution is Mn²⁺ and is in a total concentration of about 1 mM to about 30 mM, about 5 mM to about 30 mM, about 10 mM to about 30 mM, about 15 mM to about 30 mM, about 20 mM to about 30 mM, about 25 mM to about 30 mM, about 1 mM to about 25 mM, about 1 mM to about 20 mM, about 1 mM to about 15 mM, about 1 mM to about 10 mM, about 1 mM to about 5 mM, or a value within one of these ranges. Specific examples may include about 1 mM, about 2 mM, about 3 mM, about 4 mM, about 5 mM, about 6 mM, about 7 mM, about 8 mM, about 9 mM, 10 mM, about 11 mM, about 12 mM, about 13 mM, about 14 mM, about 15 mM, about 16 mM, about 17 mM, about 18 mM, about 19 mM, 20 mM, about 21 mM, about 22 mM, about 23 mM, about 24 mM, about 25 mM, about 26 mM, about 27 mM, about 28 mM, about 29 mM, about 30 mM, or a range between any two of these values.

In some embodiments, the one or more divalent cations of the first solution is Mn²⁺ and is in a total concentration of about 1 mM to about 30 mM.

In some embodiments, the one or more divalent cations of the first solution is Mn²⁺ and is in a total concentration of about 1 mM to about 10 mM.

In some embodiments, the one or more divalent cations of the first solution is Mn²⁺ and is in a total concentration of about 2 mM.

In some embodiments, the one or more divalent cations of the first solution is Cu²⁺ and is in a total concentration of about 1 mM to about 30 mM, about 5 mM to about 30 mM, about 10 mM to about 30 mM, about 15 mM to about 30 mM, about 20 mM to about 30 mM, about 25 mM to about 30 mM, about 1 mM to about 25 mM, about 1 mM to about 20 mM, about 1 mM to about 15 mM, about 1 mM to about 10 mM, about 1 mM to about 5 mM, or a value within one of these ranges. Specific examples may include about 1 mM, about 2 mM, about 3 mM, about 4 mM, about 5 mM, about 6 mM, about 7 mM, about 8 mM, about 9 mM, 10 mM, about 11 mM, about 12 mM, about 13 mM, about 14 mM, about 15 mM, about 16 mM, about 17 mM, about 18 mM, about 19 mM, 20 mM, about 21 mM, about 22 mM, about 23 mM, about 24 mM, about 25 mM, about 26 mM, about 27 mM, about 28 mM, about 29 mM, about 30 mM, or a range between any two of these values.

In some embodiments, the one or more divalent cations of the first solution is Cu²⁺ and is in a total concentration of about 1 mM to about 30 mM.

In some embodiments, the one or more divalent cations of the first solution is Cu²⁺ and is in a total concentration of about 1 mM to about 10 mM.

In some embodiments, the one or more divalent cations of the first solution is Cu²⁺ and is in a total concentration of about 2 mM.

In some embodiments, the first solution has a pH of about 5.0 to about 8.5, about 5.5 to about 8.5, about 6.0 to about 8.5, about 6.5 to about 8.5, about 7.0 to about 8.5, about 7.5 to about 8.5, about 8.0 to about 8.5, about 5.0 to about 8.0, about 5.0 to about 7.5, about 5.0 to about 7.0, about 5.0 to about 6.5, about 5.0 to about 6.0, about 5.0 to about 5.5, or a value within one of these ranges. Specific examples may include about 5.0, about 5.5, about 6.0, about 6.5, about 7.0, about 7.5, about 8.0, about 8.5, or a range between any two of these values.

In some embodiments, the first solution has a pH of about 6.0.

In some embodiments, the first solution further comprises one or more surfactants.

In some embodiments, the cation exchange column comprises a resin with a charged group wherein the charged group is sulfonate, sulfate, sulfopropyl, carboxyl, phosphate, or combinations thereof.

In some embodiments, the cation exchange column comprises a resin wherein it the resin is CaptoS, Eshmuno S, Mustang S, Poros 50HS, Poros 50 XS, S-Sepharose FF, Source S, Capto MMC, Toyopearl Gigacap S, Gigacap CM, Toyopearl SP, Toyopearl CM, MacroPrep S, UNOsphereS, MacroprepCM, Fractogel EMD SO3, Fractogel EMD COO, Fractogel EMD SE Hicap, Cellufine Sulfate, CM and SP Trisacryl, CM and S HyperD, S and CM Sepharose CL, CM Sepharose FF, S and CM CAPTO™, MonoS, Nuvia S, Cellufine phosphat, Cellufine MAX-S r, Cellufine MAX-S h, Cellufine MAX DexS-HbP, Cellufine MAX DexS-VirS, Toyopearl Sulfate 650, or Heparin Sepharose High Performance.

In some embodiments, the resin is CaptoS.

In some embodiments, the resin is Eshmuno S.

In some embodiments, the resin is Mustang S.

In some embodiments, the one or more monovalent cations of the second solution is selected from the group consisting of Na⁺, K⁺, NH₄⁺, N(C₁₋₅alkyl)₄⁺, Li⁺, Cs⁺, Cu⁺, Ag⁺, Au⁺, and combinations thereof.

In some embodiments, the one or more monovalent cations of the second solution is selected from the group consisting of Na⁺, K⁺, NH₄⁺, Li⁺, Cs⁺, and combinations thereof.

In some embodiments, the monovalent cation of the second solution is Na⁺.

In some embodiments, the one or more divalent cations of the second solution is selected from the group consisting of Ca²⁺, Mg²⁺, Zn²⁺, Mn²⁺, Cu²⁺, Fe²⁺, Ba²⁺, Sr²⁺, Co²⁺, Be²⁺, Ga²⁺, Pb²⁺, Sr²⁺, Ti²⁺, Sr²⁺, and combinations thereof.

In some embodiments, the one or more divalent cations of the second solution is selected from the group consisting of Ca²⁺, Mg²⁺, Zn²⁺, Mn²⁺, Cu²⁺, Fe²⁺, Ba²⁺, Sr²⁺, and combinations thereof.

In some embodiments, the one or more divalent cations of the second solution is selected from the group consisting of Ca²⁺, Mg²⁺, Zn²⁺, Mn²⁺, and combinations thereof.

In some embodiments, the divalent cation of the second solution is Ca²⁺.

In some embodiments, the divalent cation of the second solution is Mg²⁺.

In some embodiments, the divalent cation of the second solution is Zn²⁺.

In some embodiments, the divalent cation of the second solution is Mn²⁺.

In some embodiments, the second solution has a pH of about 5.0 to about 8.5, about 5.5 to about 8.5, about 6.0 to about 8.5, about 6.5 to about 8.5, about 7.0 to about 8.5, about 7.5 to about 8.5, about 8.0 to about 8.5, about 5.0 to about 8, about 5.0 to about 7.5, about 5.0 to about 7.0, about 5.0 to about 6.5, about 5.0 to about 6, about 5.0 to about 5.5, or a value within one of these ranges. Specific examples may include about 5, about 5.5, about 6.0, about 6.5, about 7.0, about 7.5, about 8.0, about 8.5, or a range between any two of these values.

In some embodiments, the second solution has a pH of about 6.

In some embodiments, the second solution further comprises one or more surfactants.

In some embodiments, the adding the second solution is carried out at a constant total concentration of the one or more monovalent cations.

In some embodiments, the one or more monovalent cations of the second solution is in a constant total concentration of about 5 mM to about 1500 mM, about 50 mM to about 1500 mM, about 100 mM to about 1500 mM, about 200 mM to about 1500 mM, about 300 mM to about 1500 mM, about 400 mM to about 1500 mM, about 500 mM to about 1500 mM, about 600 mM to about 1500 mM, about 700 mM to about 1500 mM, about 800 mM to about 1500 mM, about 900 mM to about 1500 mM, about 1000 mM to about 1500 mM, about 1100 mM to about 1500 mM, about 1200 mM to about 1500 mM, about 1300 mM to about 1500 mM, about 1400 mM to about 1500 mM, about 5 mM to about 1400 mM, about 5 mM to about 1300 mM, about 5 mM to about 1200 mM, about 5 mM to about 1100 mM, about 5 mM to about 1000 mM, about 5 mM to about 900 mM, about 5 mM to about 800 mM, about 5 mM to about 700 mM, about 5 mM to about 600 mM, about 5 mM to about 500 mM, about 5 mM to about 400 mM, about 5 mM to about 300 mM, about 5 mM to about 200 mM, about 5 mM to about 100 mM, about 5 mM to about 50 mM, or a value within one of these ranges. Specific examples may include about 5 mM, about 10 mM, about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, about 600 mM, about 700 mM, about 800 mM, about 900 mM, about 1000 mM, about 1100 mM, about 1200 mM, about 1300 mM, about 1400 mM, about 1500 mM, or a range between any two of these values.

In some embodiments, the one or more monovalent cations of the second solution is in a constant total concentration of about 5 mM to about 1500 mM.

In some embodiments, the one or more monovalent cations of the second solution is in a constant total concentration of about 30 mM.

In some embodiments, the adding the second solution is carried out at a constant total concentration of the Na⁺.

In some embodiments, the Na⁺ of the second solution is in a constant total concentration of about 5 mM to about 1500 mM, about 50 mM to about 1500 mM, about 100 mM to about 1500 mM, about 200 mM to about 1500 mM, about 300 mM to about 1500 mM, about 400 mM to about 1500 mM, about 500 mM to about 1500 mM, about 600 mM to about 1500 mM, about 700 mM to about 1500 mM, about 800 mM to about 1500 mM, about 900 mM to about 1500 mM, about 1000 mM to about 1500 mM, about 1100 mM to about 1500 mM, about 1200 mM to about 1500 mM, about 1300 mM to about 1500 mM, about 1400 mM to about 1500 mM, about 5 mM to about 1400 mM, about 5 mM to about 1300 mM, about 5 mM to about 1200 mM, about 5 mM to about 1100 mM, about 5 mM to about 1000 mM, about 5 mM to about 900 mM, about 5 mM to about 800 mM, about 5 mM to about 700 mM, about 5 mM to about 600 mM, about 5 mM to about 500 mM, about 5 mM to about 400 mM, about 5 mM to about 300 mM, about 5 mM to about 200 mM, about 5 mM to about 100 mM, about 5 mM to about 50 mM, or a value within one of these ranges. Specific examples may include about 5 mM, about 10 mM, about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, about 600 mM, about 700 mM, about 800 mM, about 900 mM, about 1000 mM, about 1100 mM, about 1200 mM, about 1300 mM, about 1400 mM, about 1500 mM, or a range between any two of these values.

In some embodiments, the Na⁺ of the second solution is in a constant total concentration of about 5 mM to about 1500 mM.

In some embodiments, the Na⁺ of the second solution is in a constant total concentration of about 30 mM.

In some embodiments, the adding the second solution is carried out at a constant total concentration of the one or more divalent cations.

In some embodiments, the one or more divalent cations of the second solution is in a constant total concentration of about 1 mM to about 30 mM, about 5 mM to about 30 mM, about 10 mM to about 30 mM, about 15 mM to about 30 mM, about 20 mM to about 30 mM, about 25 mM to about 30 mM, about 1 mM to about 25 mM, about 1 mM to about 20 mM, about 1 mM to about 15 mM, about 1 mM to about 10 mM, about 1 mM to about 5 mM, or a value within one of these ranges. Specific examples may include about 1 mM, about 2 mM, about 3 mM, about 4 mM, about 5 mM, about 6 mM, about 7 mM, about 8 mM, about 9 mM, 10 mM, about 11 mM, about 12 mM, about 13 mM, about 14 mM, about 15 mM, about 16 mM, about 17 mM, about 18 mM, about 19 mM, 20 mM, about 21 mM, about 22 mM, about 23 mM, about 24 mM, about 25 mM, about 26 mM, about 27 mM, about 28 mM, about 29 mM, about 30 mM, or a range between any two of these values.

In some embodiments, the one or more divalent cations of the second solution is in a constant total concentration of about 1 mM to about 30 mM.

In some embodiments, the one or more divalent cations of the second solution is in a constant total concentration of about 2 mM.

In some embodiments, the adding the second solution is carried out at a constant total concentration of the Ca²⁺.

In some embodiments, the Ca²⁺ of the second solution is in a constant total concentration of about 1 mM to about 30 mM, about 5 mM to about 30 mM, about 10 mM to about 30 mM, about 15 mM to about 30 mM, about 20 mM to about 30 mM, about 25 mM to about 30 mM, about 1 mM to about 25 mM, about 1 mM to about 20 mM, about 1 mM to about 15 mM, about 1 mM to about 10 mM, about 1 mM to about 5 mM, or a value within one of these ranges. Specific examples may include about 1 mM, about 2 mM, about 3 mM, about 4 mM, about 5 mM, about 6 mM, about 7 mM, about 8 mM, about 9 mM, 10 mM, about 11 mM, about 12 mM, about 13 mM, about 14 mM, about 15 mM, about 16 mM, about 17 mM, about 18 mM, about 19 mM, 20 mM, about 21 mM, about 22 mM, about 23 mM, about 24 mM, about 25 mM, about 26 mM, about 27 mM, about 28 mM, about 29 mM, about 30 mM, or a range between any two of these values.

In some embodiments, the Ca²⁺ of the second solution is in a constant total concentration of about 1 mM to about 30 mM.

In some embodiments, the Ca²⁺ of the second solution is in a constant total concentration of about 2 mM.

In some embodiments, the adding the second solution comprises a stepwise increase of the total concentration of the one or more monovalent cations.

In some embodiments, the initial total concentration of the one or more monovalent cations of the second solution is about 15 mM to about 60 mM, about 20 mM to about 60 mM, about 25 mM to about 60 mM, about 30 mM to about 60 mM, about 35 mM to about 60 mM, about 40 mM to about 60 mM, about 45 mM to about 60 mM, about 50 mM to about 60 mM, about 55 mM to about 60 mM, about 15 mM to about 55 mM, about 15 mM to about 50 mM, about 15 mM to about 45 mM, about 15 mM to about 40 mM, about 15 mM to about 35 mM, about 15 mM to about 30 mM, about 15 mM to about 25 mM, about 15 mM to about 20 mM, or a value within one of these ranges. Specific examples may include about 15 mM, about 20 mM, about 25 mM, about 30 mM, about 35 mM, about 40 mM, about 45 mM, about 50 mM, about 55 mM, about 60 mM, or a range between any two of these values.

In some embodiments, the initial total concentration of the one or more monovalent cations of the second solution is about 15 mM to about 60 mM.

In some embodiments, the initial total concentration of the one or more monovalent cations of the second solution is about 30 mM.

In some embodiments, the intermediate total concentration of the one or more monovalent cations of the second solution is about 100 mM to about 300 mM, about 125 mM to about 300 mM, about 150 mM to about 300 mM, about 175 mM to about 300 mM, about 200 mM to about 300 mM, about 225 mM to about 300 mM, about 250 mM to about 300 mM, about 275 mM to about 300 mM, about 100 mM to about 275 mM, about 100 mM to about 250 mM, about 100 mM to about 225 mM, about 100 mM to about 200 mM, about 100 mM to about 175 mM, about 100 mM to about 150 mM, about 100 mM to about 125 mM, or a value within one of these ranges. Specific examples may include about 100 mM, about 125 mM, about 150 mM, about 175 mM, about 200 mM, about 225 mM, about 250 mM, about 275 mM, about 300 mM, or a range between any two of these values.

In some embodiments, the intermediate total concentration of the one or more monovalent cations of the second solution is about 100 mM to about 300 mM.

In some embodiments, the intermediate total concentration of the one or more monovalent cations of the second solution is about 200 mM.

In some embodiments, the final total concentration of the one or more monovalent cations of the second solution is about 500 mM to about 1500 mM, about 600 mM to about 1500 mM, about 700 mM to about 1500 mM, about 800 mM to about 1500 mM, about 900 mM to about 1500 mM, about 1000 mM to about 1500 mM, about 1100 mM to about 1500 mM, about 1200 mM to about 1500 mM, about 1300 mM to about 1500 mM, about 1400 mM to about 1500 mM, about 500 mM to about 1400 mM, about 500 mM to about 1300 mM, about 500 mM to about 1200 mM, about 500 mM to about 1100 mM, about 500 mM to about 1000 mM, about 500 mM to about 900 mM, about 500 mM to about 800 mM, about 500 mM to about 700 mM, about 500 mM to about 600 mM, or a value within one of these ranges. Specific examples may include about 500 mM, about 600 mM, about 700 mM, about 800 mM, about 900 mM, about 1000 mM, about 1100 mM, about 1200 mM, about 1300 mM, about 1400 mM, about 1500 mM, or a range between any two of these values.

In some embodiments, the final total concentration of the one or more monovalent cations of the second solution is about 500 mM to about 1500 mM.

In some embodiments, the final total concentration of the one or more monovalent cations of the second solution is about 1000 mM.

In some embodiments, the adding the second solution comprises a stepwise increase of the total concentration of the Na⁺.

In some embodiments, the initial total concentration of the Na⁺ of the second solution is about 15 mM to about 60 mM, about 20 mM to about 60 mM, about 25 mM to about 60 mM, about 30 mM to about 60 mM, about 35 mM to about 60 mM, about 40 mM to about 60 mM, about 45 mM to about 60 mM, about 50 mM to about 60 mM, about 55 mM to about 60 mM, about 15 mM to about 55 mM, about 15 mM to about 50 mM, about 15 mM to about 45 mM, about 15 mM to about 40 mM, about 15 mM to about 35 mM, about 15 mM to about 30 mM, about 15 mM to about 25 mM, about 15 mM to about 20 mM, or a value within one of these ranges. Specific examples may include about 15 mM, about 20 mM, about 25 mM, about 30 mM, about 35 mM, about 40 mM, about 45 mM, about 50 mM, about 55 mM, about 60 mM, or a range between any two of these values.

In some embodiments, the initial total concentration of the Na⁺ of the second solution is about 15 mM to about 60 mM.

In some embodiments, the initial total concentration of the Na⁺ of the second solution is about 30 mM.

In some embodiments, the intermediate total concentration of the Na⁺ of the second solution is about 100 mM to about 300 mM, about 125 mM to about 300 mM, about 150 mM to about 300 mM, about 175 mM to about 300 mM, about 200 mM to about 300 mM, about 225 mM to about 300 mM, about 250 mM to about 300 mM, about 275 mM to about 300 mM, about 100 mM to about 275 mM, about 100 mM to about 250 mM, about 100 mM to about 225 mM, about 100 mM to about 200 mM, about 100 mM to about 175 mM, about 100 mM to about 150 mM, about 100 mM to about 125 mM, or a value within one of these ranges. Specific examples may include about 100 mM, about 125 mM, about 150 mM, about 175 mM, about 200 mM, about 225 mM, about 250 mM, about 275 mM, about 300 mM, or a range between any two of these values.

In some embodiments, the intermediate total concentration of Na⁺ of the second solution is about 100 mM to about 300 mM.

In some embodiments, the intermediate total concentration of the Na⁺ of the second solution is about 200 mM.

In some embodiments, the final total concentration of the Na⁺ of the second solution is about 500 mM to about 1500 mM, about 600 mM to about 1500 mM, about 700 mM to about 1500 mM, about 800 mM to about 1500 mM, about 900 mM to about 1500 mM, about 1000 mM to about 1500 mM, about 1100 mM to about 1500 mM, about 1200 mM to about 1500 mM, about 1300 mM to about 1500 mM, about 1400 mM to about 1500 mM, about 500 mM to about 1400 mM, about 500 mM to about 1300 mM, about 500 mM to about 1200 mM, about 500 mM to about 1100 mM, about 500 mM to about 1000 mM, about 500 mM to about 900 mM, about 500 mM to about 800 mM, about 500 mM to about 700 mM, about 500 mM to about 600 mM, or a value within one of these ranges. Specific examples may include about 500 mM, about 600 mM, about 700 mM, about 800 mM, about 900 mM, about 1000 mM, about 1100 mM, about 1200 mM, about 1300 mM, about 1400 mM, about 1500 mM, or a range between any two of these values.

In some embodiments, the final total concentration of the Na⁺ of the second solution is about 500 mM to about 1500 mM.

In some embodiments, the final total concentration of the Na⁺ of the second solution is about 1000 mM.

In some embodiments, the adding the second solution comprises a stepwise increase of the total concentration of the one or more divalent cations.

In some embodiments, the initial total concentration of the one or more divalent cations of the second solution is about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, about 9 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, about 1 mM to about 2 mM, or a value within one of these ranges. Specific examples may include about 1 mM, about 2 mM, about 3 mM, about 4 mM, about 5 mM, about 6 mM, about 7 mM, about 8 mM, about 9 mM, about 10 mM, or a range between any two of these values.

In some embodiments, the initial total concentration of the one or more divalent cations of the second solution is about 1 mM to about 10 mM.

In some embodiments, the intermediate total concentration of the one or more divalent cations of the second solution is about 10 mM to about 20 mM, about 11 mM to about 20 mM, about 12 mM to about 20 mM, about 13 mM to about 20 mM, about 14 mM to about 20 mM, about 15 mM to about 20 mM, about 16 mM to about 20 mM, about 17 mM to about 20 mM, about 18 mM to about 20 mM, about 19 mM to about 20 mM, about 10 mM to about 19 mM, about 10 mM to about 18 mM, about 10 mM to about 17 mM, about 10 mM to about 16 mM, about 10 mM to about 15 mM, about 10 mM to about 14 mM, about 10 mM to about 13 mM, about 10 mM to about 12 mM, about 10 mM to about 11 mM, or a value within one of these ranges. Specific examples may include about 10 mM, about 11 mM, about 12 mM, about 13 mM, about 14 mM, about 15 mM, about 16 mM, about 17 mM, about 18 mM, about 19 mM, about 20 mM, or a range between any two of these values.

In some embodiments, the intermediate total concentration of the one or more divalent cations of the second solution is about 10 mM to about 20 mM.

In some embodiments, the final total concentration of the one or more divalent cations of the second solution is about 20 mM to about 30 mM, about 21 mM to about 30 mM, about 22 mM to about 30 mM, about 23 mM to about 30 mM, about 24 mM to about 30 mM, about 25 mM to about 30 mM, about 26 mM to about 30 mM, about 27 mM to about 30 mM, about 28 mM to about 30 mM, about 29 mM to about 30 mM, about 20 mM to about 29 mM, about 20 mM to about 28 mM, about 20 mM to about 27 mM, about 20 mM to about 26 mM, about 20 mM to about 25 mM, about 20 mM to about 24 mM, about 20 mM to about 23 mM, about 20 mM to about 22 mM, about 20 mM to about 21 mM, or a value within one of these ranges. Specific examples may include about 20 mM, about 21 mM, about 22 mM, about 23, about 24 mM, about 25 mM, about 26 mM, about 27 mM, about 28 mM, about 29 mM, about 30 mM, or a range between any two of these values.

In some embodiments, the final total concentration of the one or more divalent cations of the second solution is about 20 mM to about 30 mM.

In some embodiments, the adding the second solution comprises a stepwise increase of the total concentration of the Ca²⁺.

In some embodiments, the initial total concentration of the Ca²⁺ of the second solution is about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, about 9 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, about 1 mM to about 2 mM, or a value within one of these ranges. Specific examples may include about 1 mM, about 2 mM, about 3 mM, about 4 mM, about 5 mM, about 6 mM, about 7 mM, about 8 mM, about 9 mM, about 10 mM, or a range between any two of these values.

In some embodiments, the initial total concentration of the Ca²⁺ of the second solution is about 1 mM to about 10 mM.

In some embodiments, the intermediate total concentration of the Ca²⁺ of the second solution is about 10 mM to about 20 mM, about 11 mM to about 20 mM, about 12 mM to about 20 mM, about 13 mM to about 20 mM, about 14 mM to about 20 mM, about 15 mM to about 20 mM, about 16 mM to about 20 mM, about 17 mM to about 20 mM, about 18 mM to about 20 mM, about 19 mM to about 20 mM, about 10 mM to about 19 mM, about 10 mM to about 18 mM, about 10 mM to about 17 mM, about 10 mM to about 16 mM, about 10 mM to about 15 mM, about 10 mM to about 14 mM, about 10 mM to about 13 mM, about 10 mM to about 12 mM, about 10 mM to about 11 mM, or a value within one of these ranges. Specific examples may include about 10 mM, about 11 mM, about 12 mM, about 13 mM, about 14 mM, about 15 mM, about 16 mM, about 17 mM, about 18 mM, about 19 mM, about 20 mM, or a range between any two of these values.

In some embodiments, the intermediate total concentration of the Ca²⁺ of the second solution is about 10 mM to about 20 mM.

In some embodiments, the final total concentration of the Ca²⁺ of the second solution is about 20 mM to about 30 mM, about 21 mM to about 30 mM, about 22 mM to about 30 mM, about 23 mM to about 30 mM, about 24 mM to about 30 mM, about 25 mM to about 30 mM, about 26 mM to about 30 mM, about 27 mM to about 30 mM, about 28 mM to about 30 mM, about 29 mM to about 30 mM, about 20 mM to about 29 mM, about 20 mM to about 28 mM, about 20 mM to about 27 mM, about 20 mM to about 26 mM, about 20 mM to about 25 mM, about 20 mM to about 24 mM, about 20 mM to about 23 mM, about 20 mM to about 22 mM, about 20 mM to about 21 mM, or a value within one of these ranges. Specific examples may include about 20 mM, about 21 mM, about 22 mM, about 23, about 24 mM, about 25 mM, about 26 mM, about 27 mM, about 28 mM, about 29 mM, about 30 mM, or a range between any two of these values.

In some embodiments, the final total concentration of the Ca²⁺ of the second solution is about 20 mM to about 30 mM.

In some embodiments, the adding the second solution comprises a continuous linear increase of the total concentration of the one or more monovalent cations.

In some embodiments, the continuous linear increase of the total concentration of the one or more monovalent cations of the second solution is about 30 mM to about 200 mM in 40 column volumes, about 40 mM to about 200 mM in 40 column volumes, about 50 mM to about 200 mM in 40 column volumes, about 75 mM to about 200 mM in 40 column volumes, about 100 mM to about 200 mM in 40 column volumes, about 125 mM to about 200 mM in 40 column volumes, about 150 mM to about 200 mM in 40 column volumes, about 175 mM to about 200 mM in 40 column volumes, about 30 mM to about 175 mM in 40 column volumes, about 30 mM to about 200 mM in 150 column volumes, about 30 mM to about 125 mM in 40 column volumes, about 30 mM to about 100 mM in 40 column volumes, about 30 mM to about 80 mM in 40 column volumes, about 30 mM to about 75 mM in 40 column volumes, about 30 mM to about 50 mM in 40 column volumes, about 30 mM to about 40 mM in 40 column volumes, or a value within one of these ranges.

In some embodiments, the continuous linear increase of the total concentration of the one or more monovalent cations of the second solution is about 30 mM to about 200 mM in 30 column volumes, about 40 mM to about 200 mM in 30 column volumes, about 50 mM to about 200 mM in 30 column volumes, about 75 mM to about 200 mM in 30 column volumes, about 100 mM to about 200 mM in 30 column volumes, about 125 mM to about 200 mM in 30 column volumes, about 150 mM to about 200 mM in 30 column volumes, about 175 mM to about 200 mM in 30 column volumes, about 30 mM to about 175 mM in 30 column volumes, about 30 mM to about 200 mM in 150 column volumes, about 30 mM to about 125 mM in 30 column volumes, about 30 mM to about 100 mM in 30 column volumes, about 30 mM to about 80 mM in 30 column volumes, about 30 mM to about 75 mM in 30 column volumes, about 30 mM to about 50 mM in 30 column volumes, about 30 mM to about 40 mM in 30 column volumes, or a value within one of these ranges.

In some embodiments, the continuous linear increase of the total concentration of the one or more monovalent cations of the second solution is about 30 mM to about 200 mM in 20 column volumes, about 40 mM to about 200 mM in 20 column volumes, about 50 mM to about 200 mM in 20 column volumes, about 75 mM to about 200 mM in 20 column volumes, about 100 mM to about 200 mM in 20 column volumes, about 125 mM to about 200 mM in 20 column volumes, about 150 mM to about 200 mM in 20 column volumes, about 175 mM to about 200 mM in 20 column volumes, about 30 mM to about 175 mM in 20 column volumes, about 30 mM to about 200 mM in 150 column volumes, about 30 mM to about 125 mM in 20 column volumes, about 30 mM to about 100 mM in 20 column volumes, about 30 mM to about 80 mM in 20 column volumes, about 30 mM to about 75 mM in 20 column volumes, about 30 mM to about 50 mM in 20 column volumes, about 30 mM to about 40 mM in 20 column volumes, or a value within one of these ranges.

In some embodiments, the continuous linear increase of the total concentration of the one or more monovalent cations of the second solution is about 30 mM to about 200 mM in 10 column volumes, about 40 mM to about 200 mM in 10 column volumes, about 50 mM to about 200 mM in 10 column volumes, about 75 mM to about 200 mM in 10 column volumes, about 100 mM to about 200 mM in 10 column volumes, about 125 mM to about 200 mM in 10 column volumes, about 150 mM to about 200 mM in 10 column volumes, about 175 mM to about 200 mM in 10 column volumes, about 30 mM to about 175 mM in 10 column volumes, about 30 mM to about 200 mM in 150 column volumes, about 30 mM to about 125 mM in 10 column volumes, about 30 mM to about 100 mM in 10 column volumes, about 30 mM to about 80 mM in 10 column volumes, about 30 mM to about 75 mM in 10 column volumes, about 30 mM to about 50 mM in 10 column volumes, about 30 mM to about 40 mM in 10 column volumes, or a value within one of these ranges.

In some embodiments, the continuous linear increase of the total concentration of the one or more monovalent cations of the second solution is about 30 mM to about 200 mM in 5 column volumes, about 40 mM to about 200 mM in 5 column volumes, about 50 mM to about 200 mM in 5 column volumes, about 75 mM to about 200 mM in 5 column volumes, about 100 mM to about 200 mM in 5 column volumes, about 125 mM to about 200 mM in 5 column volumes, about 150 mM to about 200 mM in 5 column volumes, about 175 mM to about 200 mM in 5 column volumes, about 30 mM to about 175 mM in 5 column volumes, about 30 mM to about 200 mM in 150 column volumes, about 30 mM to about 125 mM in 5 column volumes, about 30 mM to about 100 mM in 5 column volumes, about 30 mM to about 80 mM in 5 column volumes, about 30 mM to about 75 mM in 5 column volumes, about 30 mM to about 50 mM in 5 column volumes, about 30 mM to about 40 mM in 5 column volumes, or a value within one of these ranges.

In some embodiments, the continuous linear increase of the total concentration of the one or more monovalent cations of the second solution is about 30 mM to about 200 mM in 40 column volumes.

In some embodiments, the continuous linear increase of the total concentration of the one or more monovalent cations of the second solution is about 30 mM to about 80 mM in 40 column volumes.

In some embodiments, the continuous linear increase of the total concentration of the one or more monovalent cations of the second solution is about 30 mM to about 40 mM in 40 column volumes.

In some embodiments, the continuous linear increase of the total concentration of the one or more monovalent cations of the second solution is about 30 mM to about 200 mM in 30 column volumes.

In some embodiments, the continuous linear increase of the total concentration of the one or more monovalent cations of the second solution is about 30 mM to about 80 mM in 30 column volumes.

In some embodiments, the continuous linear increase of the total concentration of the one or more monovalent cations of the second solution is about 30 mM to about 40 mM in 30 column volumes.

In some embodiments, the continuous linear increase of the total concentration of the one or more monovalent cations of the second solution is about 30 mM to about 200 mM in 20 column volumes.

In some embodiments, the continuous linear increase of the total concentration of the one or more monovalent cations of the second solution is about 30 mM to about 80 mM in 20 column volumes.

In some embodiments, the continuous linear increase of the total concentration of the one or more monovalent cations of the second solution is about 30 mM to about 40 mM in 20 column volumes.

In some embodiments, the continuous linear increase of the total concentration of the one or more monovalent cations of the second solution is about 30 mM to about 200 mM in 10 column volumes.

In some embodiments, the continuous linear increase of the total concentration of the one or more monovalent cations of the second solution is about 30 mM to about 80 mM in 10 column volumes.

In some embodiments, the continuous linear increase of the total concentration of the one or more monovalent cations of the second solution is about 30 mM to about 40 mM in 10 column volumes.

In some embodiments, the continuous linear increase of the total concentration of the one or more monovalent cations of the second solution is about 30 mM to about 200 mM in 5 column volumes.

In some embodiments, the continuous linear increase of the total concentration of the one or more monovalent cations of the second solution is about 30 mM to about 80 mM in 5 column volumes.

In some embodiments, the continuous linear increase of the total concentration of the one or more monovalent cations of the second solution is about 30 mM to about 40 mM in 5 column volumes.

In some embodiments, the amount of column volumes can be reduced once the exact separation properties of a specific construct (e.g., AAV construct) is evaluated. By way of example, but not limitation, the column volumes of the gradient can be reduced to 5 column volumes (or can be below 5 column volumes) if the difference of the molarity of the monovalent cation between equilibration buffer and elution buffer is for example 5 mM to 10 mM (e.g., gradient from 30 mM Na⁺ to 40 mM Na⁺ in 5 column volumes).

In some embodiments, the adding the second solution comprises a continuous linear increase of the total concentration of the Na⁺.

In some embodiments, the continuous linear increase of the total concentration of the Na⁺ of the second solution is about 30 mM to about 200 mM in 40 column volumes, about 40 mM to about 200 mM in 40 column volumes, about 50 mM to about 200 mM in 40 column volumes, about 75 mM to about 200 mM in 40 column volumes, about 100 mM to about 200 mM in 40 column volumes, about 125 mM to about 200 mM in 40 column volumes, about 150 mM to about 200 mM in 40 column volumes, about 175 mM to about 200 mM in 40 column volumes, about 30 mM to about 175 mM in 40 column volumes, about 30 mM to about 200 mM in 150 column volumes, about 30 mM to about 125 mM in 40 column volumes, about 30 mM to about 100 mM in 40 column volumes, about 30 mM to about 80 mM in 40 column volumes, about 30 mM to about 75 mM in 40 column volumes, about 30 mM to about 50 mM in 40 column volumes, about 30 mM to about 40 mM in 40 column volumes, or a value within one of these ranges.

In some embodiments, the continuous linear increase of the total concentration of the Na⁺ of the second solution is about 30 mM to about 200 mM in 30 column volumes, about 40 mM to about 200 mM in 30 column volumes, about 50 mM to about 200 mM in 30 column volumes, about 75 mM to about 200 mM in 30 column volumes, about 100 mM to about 200 mM in 30 column volumes, about 125 mM to about 200 mM in 30 column volumes, about 150 mM to about 200 mM in 30 column volumes, about 175 mM to about 200 mM in 30 column volumes, about 30 mM to about 175 mM in 30 column volumes, about 30 mM to about 200 mM in 150 column volumes, about 30 mM to about 125 mM in 30 column volumes, about 30 mM to about 100 mM in 30 column volumes, about 30 mM to about 80 mM in 30 column volumes, about 30 mM to about 75 mM in 30 column volumes, about 30 mM to about 50 mM in 30 column volumes, about 30 mM to about 40 mM in 30 column volumes, or a value within one of these ranges.

In some embodiments, the continuous linear increase of the total concentration of the Na⁺ of the second solution is about 30 mM to about 200 mM in 20 column volumes, about 40 mM to about 200 mM in 20 column volumes, about 50 mM to about 200 mM in 20 column volumes, about 75 mM to about 200 mM in 20 column volumes, about 100 mM to about 200 mM in 20 column volumes, about 125 mM to about 200 mM in 20 column volumes, about 150 mM to about 200 mM in 20 column volumes, about 175 mM to about 200 mM in 20 column volumes, about 30 mM to about 175 mM in 20 column volumes, about 30 mM to about 200 mM in 150 column volumes, about 30 mM to about 125 mM in 20 column volumes, about 30 mM to about 100 mM in 20 column volumes, about 30 mM to about 80 mM in 20 column volumes, about 30 mM to about 75 mM in 20 column volumes, about 30 mM to about 50 mM in 20 column volumes, about 30 mM to about 40 mM in 20 column volumes, or a value within one of these ranges.

In some embodiments, the continuous linear increase of the total concentration of the Na⁺ of the second solution is about 30 mM to about 200 mM in 10 column volumes, about 40 mM to about 200 mM in 10 column volumes, about 50 mM to about 200 mM in 10 column volumes, about 75 mM to about 200 mM in 10 column volumes, about 100 mM to about 200 mM in 10 column volumes, about 125 mM to about 200 mM in 10 column volumes, about 150 mM to about 200 mM in 10 column volumes, about 175 mM to about 200 mM in 10 column volumes, about 30 mM to about 175 mM in 10 column volumes, about 30 mM to about 200 mM in 150 column volumes, about 30 mM to about 125 mM in 10 column volumes, about 30 mM to about 100 mM in 10 column volumes, about 30 mM to about 80 mM in 10 column volumes, about 30 mM to about 75 mM in 10 column volumes, about 30 mM to about 50 mM in 10 column volumes, about 30 mM to about 40 mM in 10 column volumes, or a value within one of these ranges.

In some embodiments, the continuous linear increase of the total concentration of the Na⁺ of the second solution is about 30 mM to about 200 mM in 5 column volumes, about 40 mM to about 200 mM in 5 column volumes, about 50 mM to about 200 mM in 5 column volumes, about 75 mM to about 200 mM in 5 column volumes, about 100 mM to about 200 mM in 5 column volumes, about 125 mM to about 200 mM in 5 column volumes, about 150 mM to about 200 mM in 5 column volumes, about 175 mM to about 200 mM in 5 column volumes, about 30 mM to about 175 mM in 5 column volumes, about 30 mM to about 200 mM in 150 column volumes, about 30 mM to about 125 mM in 5 column volumes, about 30 mM to about 100 mM in 5 column volumes, about 30 mM to about 80 mM in 5 column volumes, about 30 mM to about 75 mM in 5 column volumes, about 30 mM to about 50 mM in 5 column volumes, about 30 mM to about 40 mM in 5 column volumes, or a value within one of these ranges.

In some embodiments, the continuous linear increase of the total concentration of the Na⁺ of the second solution is about 30 mM to about 200 mM in 40 column volumes.

In some embodiments, the continuous linear increase of the total concentration of the Na⁺ of the second solution is about 30 mM to about 80 mM in 40 column volumes.

In some embodiments, the continuous linear increase of the total concentration of the Na⁺ of the second solution is about 30 mM to about 40 mM in 40 column volumes.

In some embodiments, the continuous linear increase of the total concentration of the Na⁺ of the second solution is about 30 mM to about 200 mM in 30 column volumes.

In some embodiments, the continuous linear increase of the total concentration of the Na⁺ of the second solution is about 30 mM to about 80 mM in 30 column volumes.

In some embodiments, the continuous linear increase of the total concentration of the Na⁺ of the second solution is about 30 mM to about 40 mM in 30 column volumes.

In some embodiments, the continuous linear increase of the total concentration of the Na⁺ of the second solution is about 30 mM to about 200 mM in 20 column volumes.

In some embodiments, the continuous linear increase of the total concentration of the Na⁺ of the second solution is about 30 mM to about 80 mM in 20 column volumes.

In some embodiments, the continuous linear increase of the total concentration of the Na⁺ of the second solution is about 30 mM to about 40 mM in 20 column volumes.

In some embodiments, the continuous linear increase of the total concentration of the Na⁺ of the second solution is about 30 mM to about 200 mM in 10 column volumes.

In some embodiments, the continuous linear increase of the total concentration of the Na⁺ of the second solution is about 30 mM to about 80 mM in 10 column volumes.

In some embodiments, the continuous linear increase of the total concentration of the Na⁺ of the second solution is about 30 mM to about 40 0 in 10 column volumes.

In some embodiments, the continuous linear increase of the total concentration of the Na⁺ of the second solution is about 30 mM to about 200 mM in 5 column volumes.

In some embodiments, the continuous linear increase of the total concentration of the Na⁺ of the second solution is about 30 mM to about 80 mM in 5 column volumes.

In some embodiments, the continuous linear increase of the total concentration of the Na⁺ of the second solution is about 30 mM to about 40 mM in 5 column volumes.

In some embodiments, the adding the second solution comprises a continuous linear increase of the total concentration of the one or more divalent cations.

In some embodiments, the continuous linear increase of the total concentration of the one or more divalent cations of the second solution is about 1 mM to about 30 mM in 40 column volumes, 5 mM to about 30 mM in 40 column volumes, 10 mM to about 30 mM in 40 column volumes, 15 mM to about 30 mM in 40 column volumes, 20 mM to about 30 mM in 40 column volumes, 25 mM to about 30 mM in 40 column volumes, 1 mM to about 25 mM in 40 column volumes, 1 mM to about 20 mM in 40 column volumes, 1 mM to about 15 mM in 40 column volumes, 1 mM to about 10 mM in 40 column volumes, 1 mM to about 5 mM in 40 column volumes, or a value within one of these ranges.

In some embodiments, the continuous linear increase of the total concentration of the one or more divalent cations of the second solution is about 1 mM to about 30 mM in 30 column volumes, 5 mM to about 30 mM in 30 column volumes, 10 mM to about 30 mM in 30 column volumes, 15 mM to about 30 mM in 30 column volumes, 20 mM to about 30 mM in 30 column volumes, 25 mM to about 30 mM in 30 column volumes, 1 mM to about 25 mM in 30 column volumes, 1 mM to about 20 mM in 30 column volumes, 1 mM to about 15 mM in 30 column volumes, 1 mM to about 10 mM in 30 column volumes, 1 mM to about 5 mM in 30 column volumes, or a value within one of these ranges.

In some embodiments, the continuous linear increase of the total concentration of the one or more divalent cations of the second solution is about 1 mM to about 30 mM in 20 column volumes, 5 mM to about 30 mM in 20 column volumes, 10 mM to about 30 mM in 20 column volumes, 15 mM to about 30 mM in 20 column volumes, 20 mM to about 30 mM in 20 column volumes, 25 mM to about 30 mM in 20 column volumes, 1 mM to about 25 mM in 20 column volumes, 1 mM to about 20 mM in 20 column volumes, 1 mM to about 15 mM in 20 column volumes, 1 mM to about 10 mM in 20 column volumes, 1 mM to about 5 mM in 20 column volumes, or a value within one of these ranges.

In some embodiments, the continuous linear increase of the total concentration of the one or more divalent cations of the second solution is about 1 mM to about 30 mM in 10 column volumes, 5 mM to about 30 mM in 10 column volumes, 10 mM to about 30 mM in 10 column volumes, 15 mM to about 30 mM in 10 column volumes, 20 mM to about 30 mM in 10 column volumes, 25 mM to about 30 mM in 10 column volumes, 1 mM to about 25 mM in 10 column volumes, 1 mM to about 20 mM in 10 column volumes, 1 mM to about 15 mM in 10 column volumes, 1 mM to about 10 mM in 10 column volumes, 1 mM to about 5 mM in 10 column volumes, or a value within one of these ranges.

In some embodiments, the continuous linear increase of the total concentration of the one or more divalent cations of the second solution is about 1 mM to about 30 mM in 5 column volumes, 5 mM to about 30 mM in 5 column volumes, 10 mM to about 30 mM in 5 column volumes, 15 mM to about 30 mM in 5 column volumes, 20 mM to about 30 mM in 5 column volumes, 25 mM to about 30 mM in 5 column volumes, 1 mM to about 25 mM in 5 column volumes, 1 mM to about 20 mM in 5 column volumes, 1 mM to about 15 mM in 5 column volumes, 1 mM to about 10 mM in 5 column volumes, 1 mM to about 5 mM in 5 column volumes, or a value within one of these ranges.

In some embodiments, the continuous linear increase of the total concentration of the one or more divalent cations of the second solution is about 1 mM to about 30 mM in 40 column volumes.

In some embodiments, the continuous linear increase of the total concentration of the one or more divalent cations of the second solution is about 1 mM to about 15 mM in 40 column volumes.

In some embodiments, the continuous linear increase of the total concentration of the one or more divalent cations of the second solution is about 1 mM to about 5 mM in 40 column volumes.

In some embodiments, the continuous linear increase of the total concentration of the one or more divalent cations of the second solution is about 1 mM to about 30 mM in 30 column volumes.

In some embodiments, the continuous linear increase of the total concentration of the one or more divalent cations of the second solution is about 1 mM to about 15 mM in 30 column volumes.

In some embodiments, the continuous linear increase of the total concentration of the one or more divalent cations of the second solution is about 1 mM to about 5 mM in 30 column volumes.

In some embodiments, the continuous linear increase of the total concentration of the one or more divalent cations of the second solution is about 1 mM to about 30 mM in 20 column volumes.

In some embodiments, the continuous linear increase of the total concentration of the one or more divalent cations of the second solution is about 1 mM to about 15 mM in 20 column volumes.

In some embodiments, the continuous linear increase of the total concentration of the one or more divalent cations of the second solution is about 1 mM to about 5 mM in 20 column volumes.

In some embodiments, the continuous linear increase of the total concentration of the one or more divalent cations of the second solution is about 1 mM to about 30 mM in 10 column volumes.

In some embodiments, the continuous linear increase of the total concentration of the one or more divalent cations of the second solution is about 1 mM to about 15 mM in 10 column volumes.

In some embodiments, the continuous linear increase of the total concentration of the one or more divalent cations of the second solution is about 1 mM to about 5 mM in 10 column volumes.

In some embodiments, the continuous linear increase of the total concentration of the one or more divalent cations of the second solution is about 1 mM to about 30 mM in 5 column volumes.

In some embodiments, the continuous linear increase of the total concentration of the one or more divalent cations of the second solution is about 1 mM to about 15 mM in 5 column volumes.

In some embodiments, the continuous linear increase of the total concentration of the one or more divalent cations of the second solution is about 1 mM to about 5 mM in 5 column volumes.

In some embodiments, the adding the second solution comprises a continuous linear increase of the total concentration of the Ca²⁺.

In some embodiments, the continuous linear increase of the total concentration of the Ca²⁺ of the second solution is about 1 mM to about 30 mM in 40 column volumes, 5 mM to about 30 mM in 40 column volumes, 10 mM to about 30 mM in 40 column volumes, 15 mM to about 30 mM in 40 column volumes, 20 mM to about 30 mM in 40 column volumes, 25 mM to about 30 mM in 40 column volumes, 1 mM to about 25 mM in 40 column volumes, 1 mM to about 20 mM in 40 column volumes, 1 mM to about 15 mM in 40 column volumes, 1 mM to about 10 mM in 40 column volumes, 1 mM to about 5 mM in 40 column volumes, or a value within one of these ranges.

In some embodiments, the continuous linear increase of the total concentration of the Ca²⁺ of the second solution is about 1 mM to about 30 mM in 30 column volumes, 5 mM to about 30 mM in 30 column volumes, 10 mM to about 30 mM in 30 column volumes, 15 mM to about 30 mM in 30 column volumes, 20 mM to about 30 mM in 30 column volumes, 25 mM to about 30 mM in 30 column volumes, 1 mM to about 25 mM in 30 column volumes, 1 mM to about 20 mM in 30 column volumes, 1 mM to about 15 mM in 30 column volumes, 1 mM to about 10 mM in 30 column volumes, 1 mM to about 5 mM in 30 column volumes, or a value within one of these ranges.

In some embodiments, the continuous linear increase of the total concentration of the Ca²⁺ of the second solution is about 1 mM to about 30 mM in 20 column volumes, 5 mM to about 30 mM in 20 column volumes, 10 mM to about 30 mM in 20 column volumes, 15 mM to about 30 mM in 20 column volumes, 20 mM to about 30 mM in 20 column volumes, 25 mM to about 30 mM in 20 column volumes, 1 mM to about 25 mM in 20 column volumes, 1 mM to about 20 mM in 20 column volumes, 1 mM to about 15 mM in 20 column volumes, 1 mM to about 10 mM in 20 column volumes, 1 mM to about 5 mM in 20 column volumes, or a value within one of these ranges.

In some embodiments, the continuous linear increase of the total concentration of the Ca²⁺ of the second solution is about 1 mM to about 30 mM in 10 column volumes, 5 mM to about 30 mM in 10 column volumes, 10 mM to about 30 mM in 10 column volumes, 15 mM to about 30 mM in 10 column volumes, 20 mM to about 30 mM in 10 column volumes, 25 mM to about 30 mM in 10 column volumes, 1 mM to about 25 mM in 10 column volumes, 1 mM to about 20 mM in 10 column volumes, 1 mM to about 15 mM in 10 column volumes, 1 mM to about 10 mM in 10 column volumes, 1 mM to about 5 mM in 10 column volumes, or a value within one of these ranges.

In some embodiments, the continuous linear increase of the total concentration of the Ca²⁺ of the second solution is about 1 mM to about 30 mM in 5 column volumes, 5 mM to about 30 mM in 5 column volumes, 10 mM to about 30 mM in 5 column volumes, 15 mM to about 30 mM in 5 column volumes, 20 mM to about 30 mM in 5 column volumes, 25 mM to about 30 mM in 5 column volumes, 1 mM to about 25 mM in 5 column volumes, 1 mM to about 20 mM in 5 column volumes, 1 mM to about 15 mM in 5 column volumes, 1 mM to about 10 mM in 5 column volumes, 1 mM to about 5 mM in 5 column volumes, or a value within one of these ranges.

In some embodiments, the continuous linear increase of the total concentration of the total concentration of the Ca²⁺ of the second solution is about 1 mM to about 30 mM in 40 column volumes.

In some embodiments, the continuous linear increase of the total concentration of the Ca²⁺ of the second solution is about 1 mM to about 15 mM in 40 column volumes.

In some embodiments, the continuous linear increase of the total concentration of the Ca²⁺ of the second solution is about 1 mM to about 5 mM in 40 column volumes.

In some embodiments, the continuous linear increase of the total concentration of the total concentration of the Ca²⁺ of the second solution is about 1 mM to about 30 mM in 30 column volumes.

In some embodiments, the continuous linear increase of the total concentration of the Ca²⁺ of the second solution is about 1 mM to about 15 mM in 30 column volumes.

In some embodiments, the continuous linear increase of the total concentration of the Ca²⁺ of the second solution is about 1 mM to about 5 mM in 30 column volumes.

In some embodiments, the continuous linear increase of the total concentration of the total concentration of the Ca²⁺ of the second solution is about 1 mM to about 30 mM in 20 column volumes.

In some embodiments, the continuous linear increase of the total concentration of the Ca²⁺ of the second solution is about 1 mM to about 15 mM in 20 column volumes.

In some embodiments, the continuous linear increase of the total concentration of the Ca²⁺ of the second solution is about 1 mM to about 5 mM in 20 column volumes.

In some embodiments, the continuous linear increase of the total concentration of the total concentration of the Ca²⁺ of the second solution is about 1 mM to about 30 mM in 0 column volumes.

In some embodiments, the continuous linear increase of the total concentration of the Ca²⁺ of the second solution is about 1 mM to about 15 mM in 0 column volumes.

In some embodiments, the continuous linear increase of the total concentration of the Ca²⁺ of the second solution is about 1 mM to about 5 mM in 0 column volumes.

In some embodiments, the continuous linear increase of the total concentration of the total concentration of the Ca²⁺ of the second solution is about 1 mM to about 30 mM in 5 column volumes.

In some embodiments, the continuous linear increase of the total concentration of the Ca²⁺ of the second solution is about 1 mM to about 15 mM in 5 column volumes.

In some embodiments, the continuous linear increase of the total concentration of the Ca²⁺ of the second solution is about 1 mM to about 5 mM in 5 column volumes.

In some embodiments, the AAV capsid is derived from the group consisting of AAV2, AAV3, AAV3b, AAV4, AAV5, AAV6, AAV7, AAV8, AAV9, AAV10, AAV11, AAV12, genetical modified AAV, chemical modified AAV, genetical and chemical modified AAV, and combinations thereof.

In some embodiments, the AAV capsid is derived from AAV8.

In some embodiments, the AAV capsid is derived from AAV9.

In some embodiments, the AAV capsid is derived from AAV6.

In some embodiments, the one or more surfactants (e.g., first solution or second solution) is selected from the group consisting of polysorbate 20, polysorbate 40, polysorbate 65, polysorbate 80, polyoxyethylene glycol tert-octylphenol ether, sorbitan monolaurate, sorbitan monopalmitate, sorbitan monostearate, sorbitan tristearate, sorbitan monooleate, sorbitan trioleate, polyoxyethylene (20) sorbitan monopalmitate, polyoxyethylene (20) sorbitan monostearate, polyoxyethylene (20) sorbitan tristearate, polyoxyethylene (20) sorbitan trioleate, polyoxyethylen(20)-sorbitan-monooleate (Tween 80/Polysorbate 80)), poloxamer 124, poloxamer 188, poloxamer 407, cremophor, Triton N-101 reduced, Triton X-100, and combinations thereof.

In some embodiments, the one or more surfactants (e.g., first solution or second solution) is selected from the group consisting of polysorbate 20, polysorbate 80, poloxamer 124, and combinations thereof.

In some embodiments, the one or more surfactants (e.g., first solution or second solution) is polysorbate 80.

In some embodiments, the one or more surfactants (e.g., first solution or second solution) is polysorbate 20.

In some embodiments, the one or more surfactants (e.g., first solution or second solution) is polysorbate 124.

In some embodiments, the one or more surfactants (e.g., first solution or second solution) is in a total amount of about 0.0025 w/w % to about 0.0075 w/w %, about 0.003 w/w % to about 0.0075 w/w %, about 0.0035 w/w % to about 0.0075 w/w %, about 0.004 w/w % to about 0.0075 w/w %, about 0.0045 w/w % to about 0.0075 w/w %, about 0.005 w/w % to about 0.0075 w/w %, about 0.0055 w/w % to about 0.0075 w/w %, about 0.006 w/w % to about 0.0075 w/w %, about 0.0065 w/w % to about 0.0075 w/w %, about 0.007 w/w % to about 0.0075 w/w %, about 0.0025 w/w % to about 0.0070 w/w %, about 0.0025 w/w % to about 0.0065 w/w %, about 0.0025 w/w % to about 0.006 w/w %, about 0.0025 w/w % to about 0.0065 w/w %, about 0.0025 w/w % to about 0.006 w/w %, about 0.0025 w/w % to about 0.0055 w/w %, about 0.0025 w/w % to about 0.005 w/w %, about 0.0025 w/w % to about 0.0045 w/w %, about 0.0025 w/w % to about 0.004 w/w %, about 0.0025 w/w % to about 0.0035 w/w %, about 0.0025 w/w % to about 0.003 w/w %, or a value within one of these ranges. Specific examples may include about 0.0025 w/w %, about 0.003 w/w %, about 0.0035 w/w %, about 0.004 w/w %, about 0.0045 w/w %, about 0.005 w/w %, about 0.0055 w/w %, about 0.006 w/w %, about 0.0065 w/w %, about 0.007 w/w %, about 0.0075 w/w %, or a range between any two of these values.

In some embodiments, the one or more surfactants (e.g., first solution or second solution) is in a total amount of about 0.005 w/w %.

In certain embodiments, the method for purifying empty AAV capsids from an AAV preparation or fraction comprising empty AAV capsids and full AAV capsids, to provide an AAV product, formulation, or composition substantially free of full AAV capsids comprising the steps of:
(a) providing a first solution comprising empty AAV capsids, full AAV capsids, one or more monovalent cations, and one or more divalent cations;
(b) loading said first solution onto a cation exchange column under conditions whereby said empty AAV capsids and said full AAV capsids bind to the column; and
(c) adding a second solution comprising one or more monovalent cations and one or more divalent cations to the cation exchange column under conditions wherein the empty AAV capsids are purified from the full AAV capsids.

In some embodiments, the purified AAV product, formulation, or composition comprises less than about 50%, about 45%, about 40%, about 35%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% of empty AAV capsids. In some embodiments, the purified AAV product, formulation, or composition comprises less than about 30% of empty AAV capsids. In some embodiments, the purified AAV product, formulation, or composition comprises less than about 20% of empty AAV capsids. In some embodiments, the purified AAV product, formulation, or composition comprises less than about 6% of empty AAV capsids.

In certain embodiments, the method of separating empty AAV capsids and full AAV capsids in an AAV preparation or fraction comprising the steps of:
(a) providing a first solution comprising empty AAV capsids, full AAV capsids, one or more monovalent cations, and one or more divalent cations;
(b) loading said first solution onto a cation exchange column under conditions whereby said empty AAV capsids and said full AAV capsids bind to the column; and
(c) adding a second solution comprising one or more monovalent cations and one or more divalent cations to the cation exchange column under conditions wherein the empty AAV capsids are separated from the full AAV capsids.

In certain embodiments, at the low conductivity zone the full capsids or substantially full capsids can be selected and in the higher conductivity zone the empty capsids or substantially empty capsids can be selected.

In some embodiments, the empty AAV capsid is derived from the group consisting of AAV2, AAV3, AAV3b, AAV4, AAV5, AAV6, AAV7, AAV8, AAV9, AAV10, AAV11, AAV12, genetical modified AAV, chemical modified AAV, genetical and chemical modified AAV, and combinations thereof.

In some embodiments, the empty AAV capsid is derived from AAV8.

In some embodiments, the empty AAV capsid is derived from AAV9.

In some embodiments, the empty AAV capsid is derived from AAV6.

Some embodiments are directed towards a method of preparing an immune absorption column comprising the steps of
(a) concentrating the empty AAV capsids of any embodiments disclosed herein by ultrafiltration;
(b) applying a buffer exchange into an amine free buffer; and
(c) immobilizing the empty AAV capsids on an activated resin.

Some embodiments are directed towards a method of preparing an immune absorption column comprising the steps of
(a) concentrating the empty AAV capsids of any embodiments disclosed herein by anion exchanger;
(b) applying a buffer exchange into an amine free buffer; and
(c) immobilizing the empty AAV capsids on an activated resin.

Some embodiments are directed towards a method of preparing an immune absorption column comprising the steps of
(a) concentrating the empty AAV capsids of any embodiments disclosed herein a cation exchanger;
(b) applying a buffer exchange into an amine free buffer; and
(c) immobilizing the empty AAV capsids on an activated resin.

Some embodiments are directed towards a method of preparing an immune absorption column comprising the steps of
(a) concentrating the full AAV capsids of any embodiments disclosed herein by ultrafiltration; and
(b) applying a buffer exchange into a buffer suitable for treating a patient.

A method of preparing an immune absorption column comprising the steps of
(a) concentrating the full AAV capsids of any embodiments disclosed herein by anion exchanger; and
(b) applying a buffer exchange into a buffer suitable for treating a patient.

Some embodiments are directed towards a method of preparing an immune absorption column comprising the steps of
(a) concentrating the full AAV capsids of any embodiments disclosed herein a cation exchanger; and
(b) applying a buffer exchange into a buffer suitable for treating a patient.

In some embodiments the immobilizing the empty AAV capsids on an activated resin occurs at a temperature of about 2° C. to about 37° C., about 5° C. to about 37° C., about 10° C. to about 37° C., about 15° C. to about 37° C., about 20° C. to about 37° C., about 25° C. to about 37° C., about 30° C. to about 37° C., about 35° C. to about 37° C., about 2° C. to about 35° C., about 2° C. to about 30° C., about 2° C. to about 25° C., about 2° C. to about 20° C., about 2° C. to about 15° C., about 2° C. to about 10° C., about 2° C. to about 5° C., about 5° C. to about 35° C., about 10° C. to about 30° C., about 15° C. to about 25° C., or a value within one of these ranges. Specific examples may include about 2° C., about 5° C., about 10° C., about 15° C., about 20° C., about 25° C., about 30° C., about 35° C., about 37° C., or a range between any two of these values.

In some embodiments the immobilizing the empty AAV capsids on an activated resin is carried out with an reaction time of about 2 hours (hrs) to about 20 hrs, about 2 hrs to about 19 hrs, about 2 hrs to about 18 hrs, about 2 hrs to about 17 hrs, about 2 hrs to about 16 hrs, about 2 hrs to about 15 hrs, about 2 hrs to about 14 hrs, about 2 hrs to about 13 hrs, about 2 hrs to about 12 hrs, about 2 hrs to about 11 hrs, about 2 hrs to about 10 hrs, about 2 hrs to about 9 hrs, about 2 hrs to about 8 hrs, about 2 hrs to about 7 hrs, about 2 hrs to about 6 hrs, about 2 hrs to about 5 hrs, about 2 hrs to about 4 hrs, about 2 hrs to about 3 hrs, about 3 hrs to about 20 hrs, about 4 hrs to about 20 hrs, about 5 hrs to about 20 hrs, about 6 hrs to about 20 hrs, about 7 hrs to about 20 hrs, about 8 hrs to about 20 hrs, about 9 hrs to about 20 hrs, about 10 hrs to about 20 hrs, about 11 hrs to about 20 hrs, about 12 hrs to about 20 hrs, about 13 hrs to about 20 hrs, about 14 hrs to about 20 hrs, about 15 hrs to about 20 hrs, about 16 hrs to about 20 hrs, about 17 hrs to about 20 hrs, about 18 hrs to about 20 hrs, about 19 hrs to about 20 hrs, or a value within one of these ranges. Specific examples may include about 2 hrs, about 3 hrs, about 4 hrs, about 5 hrs, about 6 hrs, about 7 hrs, about 8 hrs, about 9 hrs, about 10 hrs, about 11 hrs, about 12 hrs, about 13 hrs, about 14 hrs, about 15 hrs, about 16 hrs, about 17 hrs, about 18 hrs, about 19 hrs, about 20 hrs, or a range between any two of these values.

In some embodiments the amine free buffer is selected from the group consisting of a phosphate buffer, a citrate buffer, a carbonate buffer, an acetate buffer, a borate buffer, 4-(2-hydroxyethyl)-1-piperazineethanesulfonic acid (HEPES), and combinations thereof.

In some embodiments the amine free buffer is in a concentration of about 50 mM to about 150 mM, about 50 mM to about 125 mM, about 50 mM to about 100 mM, about 75 mM to about 150 mM, about 100 mM to about 150 mM, about 75 mM to about 125 mM, or a value within one of these ranges. Specific examples may include about 50 mM, about 75 mM, about 100 mM, about 125 mM, about 150 mM, or a range between any two of these values.

In some embodiments the amine free buffer is in a concentration of about 100 mM.

In some embodiments the amine free buffer further comprises NaCl.

In some embodiments the NaCl is in a concentration of about 100 mM to about 200 mM, about 125 mM to about 200 mM, about 150 mM to about 200 mM, about 100 mM to about 175 mM, about 100 mM to about 150 mM, about 125 mM to about 175 mM, or a value within one of these ranges. Specific examples may include about 100 mM, about 125 mM, about 150 mM, about 175 mM, about 200 mM, or a range between any two of these values.

In some embodiments the NaCl is in a concentration of about 150 mM.

In some embodiments the amine free buffer has a pH of about 7.0 to about 8.5, about 7.25 to about 8.5, about 7.5 to about 8.5, about 7.75 to about 8.5, about 8.0 to about 8.5, about 8.25 to about 8.5, about 7.0 to about 8.25, about 7.0 to about 8, about 7.0 to about 7.75, about 7.0 to about 7.5, about 7.0 to about 7.25, or a value within one of these ranges. Specific examples may include about 7.0, about 7.25, about 7.5, about 7.75, about 8.0, about 8.25, about 8.5, or a range between any two of these values.

In some embodiments the amine free buffer has a pH of about 8.0 to about 8.5.

In some embodiments the activated resin is selected from the group consisting of CNBr-Sepharose FF, NHS-Sepharose FF, Praesto® CNBr, Poros EP, and Poros AL.

In some embodiments the amine free buffer has a pH about 6.5, about 7, about 7.5, about 8, or a range between any two of these values.

In some embodiments the amine free buffer has a pH of about 7.5.

In some embodiments the anion exchanger is selected from the group consisting of Fractogel TMAE, Poros PI, Q Sepharose HP, Poros HQ, Toyopearl GigaCap Q 650, Cellufine Max Q, and Praesto Q.

In some embodiments the anion exchanger is selected from the group consisting of Fractogel TMAE, Poros PI, and Poros HQ.

In some embodiments the anion exchanger is Fractogel TMAE.

In some embodiments the anion exchanger is Poros PI.

In some embodiments the anion exchanger is Poros HQ.

In some embodiments the cation exchanger is selected from the group consisting of Capto S, Eshmuno S, Mustang S, cellufinesulfate, cellufinephosphate, Toyopearl sulfate 650, Poros XS, Poros HS, and Praesto SP.

In some embodiments the cation exchanger is selected from the group consisting of Capto S and Eshmuno S.

In some embodiments the cation exchanger is Capto S.

In some embodiments the cation exchanger is Eshmuno S.

By way of example and not limitation, potential buffers suitable for treating patients can be found in WO2018128689A1 and WO2020014479A1, which are incorporated herein by reference in their entirety for all intended purposes.

In some embodiments the buffer suitable for treating a patient is L-histidine.

In some embodiments the buffer suitable for treating a patient is in a concentration of about 5 mM to about 25 mM, about 5 mM to about 15 mM, about 10 mM to about 20 mM, or about 15 mM to about 25 mM, or a value within one of these ranges. Specific examples may include about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM, or a range between any two of these values.

In some embodiments the buffer suitable for treating a patient further comprises NaCl.

In some embodiments the NaCl is in a concentration of about 100 mM to about 200 mM, about 125 mM to about 200 mM, about 150 mM to about 200 mM, about 100 mM to about 175 mM, about 100 mM to about 150 mM, about 125 mM to about 175 mM, or a value within one of these ranges. Specific examples may include about 100 mM, about 125 mM, about 150 mM, about 175 mM, about 200 mM, or a range between any two of these values.

In some embodiments the NaCl is in a concentration of about 150 mM.

In some embodiments the buffer suitable for treating a patient has a pH of about 6.5 to about 9.0, about 6.5 to about 8.0, about 6.9 to about 7.7, or about 7.0 to about 7.5, or a value within one of these ranges. Specific examples may include about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, or a range between any two of these values.

In some embodiments the buffer suitable for treating a patient has a pH of about 7.0.

In some embodiments the amine free buffer has a pH of about 8.0 to about 7.5.

Some embodiments are directed toward a method for purifying empty AAV capsids from an AAV preparation or fraction, wherein the purified AAV product, formulation, composition, etc. comprises about less than 40%, about 35%, about 30%, about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% empty AAV capsids. In some embodiments, the purified AAV product, formulation, or composition comprises about 1% to about 30%, about 4% to about 30%, about 1% to about 20%, about 1% to about 6%, about 2% to about 20%, about 3% to about 20%, about 4% to about 20%, about 5% to about 20%, about 6% to about 20%, about 4% to about 19%, about 5% to about 19%, about 4% to about 12%, about 5% to about 12%, about 4% to about 11%, about, or about 5% to about 11% empty AAV capsids.

### AAV Formulations and AAV Products

Provided herein is an AAV formulation comprising full AAV capsids purified according to the method as described herein.

Also provided herein is a pharmaceutical composition comprising an AAV product produced by a method as described herein.

In exemplary embodiments, the AAV formulations or AAV compostions or AAV products of the present disclosure comprise additional pharmaceutically acceptable ingredients. In exemplary aspects, the AAV formulations or AAV compostions or AAV products comprise any one or a combination of the following: acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. In some embodiments, the AAV formulations or AAV compostions or AAV products of the present disclosure comprise any one or a combination of the following components: acacia, acesulfame potassium, acetyltributyl citrate, acetyltriethyl citrate, agar, albumin, alcohol, dehydrated alcohol, denatured alcohol, dilute alcohol, aleuritic acid, alginic acid, aliphatic polyesters, alumina, aluminum hydroxide, aluminum stearate, amylopectin, a-amylose, ascorbic acid, ascorbyl palmitate, aspartame, bacteriostatic water for injection, bentonite, bentonite magma, benzalkonium chloride, benzethonium chloride, benzoic acid, benzyl alcohol, benzyl benzoate, bronopol, butylated hydroxyanisole, butylated hydroxytoluene, butylparaben, butylparaben sodium, calcium alginate, calcium ascorbate, calcium carbonate, calcium cyclamate, dibasic anhydrous calcium phosphate, dibasic dehydrate calcium phosphate, tribasic calcium phosphate, calcium propionate, calcium silicate, calcium sorbate, calcium stearate, calcium sulfate, calcium sulfate hemihydrate, canola oil, carbomer, carbon dioxide, carboxymethyl cellulose calcium, carboxymethyl cellulose sodium, β-carotene, carrageenan, castor oil, hydrogenated castor oil, cationic emulsifying wax, cellulose acetate, cellulose acetate phthalate, ethyl cellulose, microcrystalline cellulose, powdered cellulose, silicified microcrystalline cellulose, sodium carboxymethyl cellulose, cetostearyl alcohol, cetrimide, cetyl alcohol, chlorhexidine, chlorobutanol, chlorocresol, cholesterol, chlorhexidine acetate, chlorhexidine gluconate, chlorhexidine hydrochloride, chlorodifluoroethane (HCFC), chlorodifluoromethane, chlorofluorocarbons (CFC)chlorophenoxyethanol, chloroxylenol, corn syrup solids, anhydrous citric acid, citric acid monohydrate, cocoa butter, coloring agents, corn oil, cottonseed oil, cresol, m-cresol, o-cresol, p-cresol, croscarmellose sodium, crospovidone, cyclamic acid, cyclodextrins, dextrates, dextrin, dextrose, dextrose anhydrous, diazolidinyl urea, dibutyl phthalate, dibutyl sebacate, diethanolamine, diethyl phthalate, difluoroethane (HFC), dimethyl-β-cyclodextrin, cyclodextrin-type compounds such as Captisol®, dimethyl ether, dimethyl phthalate, dipotassium edentate, disodium edentate, disodium hydrogen phosphate, docusate calcium, docusate potassium, docusate sodium, dodecyl gallate, dodecyltrimethylammonium bromide, edentate calcium disodium, edtic acid, eglumine, ethyl alcohol, ethylcellulose, ethyl gallate, ethyl laurate, ethyl maltol, ethyl oleate, ethylparaben, ethylparaben potassium, ethylparaben sodium, ethyl vanillin, fructose, fructose liquid, fructose milled, fructose pyrogen-free, powdered fructose, fumaric acid, gelatin, glucose, liquid glucose, glyceride mixtures of saturated vegetable fatty acids, glycerin, glyceryl behenate, glyceryl monooleate, glyceryl monostearate, self-emulsifying glyceryl monostearate, glyceryl palmitostearate, glycine, glycols, glycofurol, guar gum, heptafluoropropane (HFC), hexadecyltrimethylammonium bromide, high fructose syrup, human serum albumin, hydrocarbons (HC), dilute hydrochloric acid, hydrogenated vegetable oil type II, hydroxyethyl cellulose, 2-hydroxyethyl-β-cyclodextrin, hydroxypropyl cellulose, low-substituted hydroxypropyl cellulose, 2-hydroxypropyl-β-cyclodextrin, hydroxypropyl methylcellulose, hydroxypropyl methylcellulose phthalate, imidurea, indigo carmine, ion exchangers, iron oxides, isopropyl alcohol, isopropyl myristate, isopropyl palmitate, isotonic saline, kaolin, lactic acid, lactitol, lactose, lanolin, lanolin alcohols, anhydrous lanolin, lecithin, magnesium aluminum silicate, magnesium carbonate, normal magnesium carbonate, magnesium carbonate anhydrous, magnesium carbonate hydroxide, magnesium hydroxide, magnesium lauryl sulfate, magnesium oxide, magnesium silicate, magnesium stearate, magnesium trisilicate, magnesium trisilicate anhydrous, malic acid, malt, maltitol, maltitol solution, maltodextrin, maltol, maltose, mannitol, medium chain triglycerides, meglumine, menthol, methylcellulose, methyl methacrylate, methyl oleate, methylparaben, methylparaben potassium, methylparaben sodium, microcrystalline cellulose and carboxymethylcellulose sodium, mineral oil, light mineral oil, mineral oil and lanolin alcohols, oil, olive oil, monoethanolamine, montmorillonite, octyl gallate, oleic acid, palmitic acid, paraffin, peanut oil, petrolatum, petrolatum and lanolin alcohols, pharmaceutical glaze, phenol, liquified phenol, phenoxyethanol, phenoxypropanol, phenylethyl alcohol, phenylmercuric acetate, phenylmercuric borate, phenylmercuric nitrate, polacrilin, polacrilin potassium, poloxamer, polydextrose, polyethylene glycol, polyethylene oxide, polyacrylates, polyethylene-polyoxypropylene-block polymers, polymethacrylates, polyoxyethylene alkyl ethers, polyoxyethylene castor oil derivatives, polyoxyethylene sorbitol fatty acid esters, polyoxyethylene stearates, polyvinyl alcohol, polyvinyl pyrrolidone, potassium alginate, potassium benzoate, potassium bicarbonate, potassium bisulfite, potassium chloride, potassium citrate, potassium citrate anhydrous, potassium hydrogen phosphate, potassium metabisulfite, monobasic potassium phosphate, potassium propionate, potassium sorbate, povidone, propanol, propionic acid, propylene carbonate, propylene glycol, propylene glycol alginate, propyl gallate, propylparaben, propylparaben potassium, propylparaben sodium, protamine sulfate, rapeseed oil, Ringer's solution, saccharin, saccharin ammonium, saccharin calcium, saccharin sodium, safflower oil, saponite, serum proteins, sesame oil, colloidal silica, colloidal silicon dioxide, sodium alginate, sodium ascorbate, sodium benzoate, sodium bicarbonate, sodium bisulfite, sodium chloride, anhydrous sodium citrate, sodium citrate dehydrate, sodium chloride, sodium cyclamate, sodium edentate, sodium dodecyl sulfate, sodium lauryl sulfate, sodium metabisulfite, sodium phosphate, dibasic, sodium phosphate, monobasic, sodium phosphate, tribasic, anhydrous sodium propionate, sodium propionate, sodium sorbate, sodium starch glycolate, sodium stearyl fumarate, sodium sulfite, sorbic acid, sorbitan esters (sorbitan fatty esters), sorbitol, sorbitol solution 70%, soybean oil, spermaceti wax, starch, corn starch, potato starch, pregelatinized starch, sterilizable maize starch, stearic acid, purified stearic acid, stearyl alcohol, sucrose, sugars, compressible sugar, confectioner's sugar, sugar spheres, invert sugar, Sugartab, Sunset Yellow FCF, synthetic paraffin, talc, tartaric acid, tartrazine, tetrafluoroethane (HFC), theobroma oil, thimerosal, titanium dioxide, alpha tocopherol, tocopheryl acetate, alpha tocopheryl acid succinate, beta-tocopherol, delta-tocopherol, gamma-tocopherol, tragacanth, triacetin, tributyl citrate, triethanolamine, triethyl citrate, trimethyl-p-cyclodextrin, trimethyltetradecylammonium bromide, tris buffer, trisodium edentate, vanillin, type I hydrogenated vegetable oil, water, soft water, hard water, carbon dioxide-free water, pyrogen-free water, water for injection, sterile water for inhalation, sterile water for injection, sterile water for irrigation, waxes, anionic emulsifying wax, carnauba wax, cationic emulsifying wax, cetyl ester wax, microcrystalline wax, nonionic emulsifying wax, suppository wax, white wax, yellow wax, white petrolatum, wool fat, xanthan gum, xylitol, zein, zinc propionate, zinc salts, zinc stearate, or any excipient in the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety for all intended purposes, discloses various components used in formulating pharmaceutically acceptable AAV formulations or AAV products or AAV composition and known techniques for the preparation thereof. Except insofar as any conventional agent is incompatible with the pharmaceutical AAV formulations or AAV products or AAV composition, its use in pharmaceutical AAV formulations or AAV products or AAV composition is contemplated. In exemplary embodiments, the AAV formulations or AAV products or AAV composition of the present disclosure do not comprise one or a combination of the above ingredients. In exemplary embodiments, the AAV formulations or AAV products or AAV composition of the present disclosure comprise none of these ingredients. In exemplary aspects, the pharmaceutical AAV formulations or AAV products or AAV composition of the present disclosure does not comprise dextran. In exemplary aspects, the pharmaceutical AAV formulations or AAV products or AAV composition of the present disclosure does not comprise calcium chloride.

Quantitative and/or Qualitative Methods

The methods of the present disclosure comprise one or more quality control steps, e.g., steps to measure the concentration, dose, and/or potency of the AAV fractions, preparations, products, formulations, or compositions obtained after one or more steps (e.g., after each step) of the purification process, including the final step for making an AAV preparation, product, formulation, or composition for administration.

The methods of the present disclosure can comprise an ELISA assay, specific for AAV (e.g., AAV antigen), for quantitating the number of AAV capsids. In certain embodiments, the ELISA assay can include, but is not limited to, an immunosorbent assay, direct ELISA, indirect ELISA, sandwich ELISA, and/or a competitive ELISA. In certain embodiments, the ELISA is a sandwich ELISA.

In certain embodiments, the AAV antigen is an AAV1, AAV2, AAV3, AAV4, AAV5, AAV6, AAV7, AAV8, AAV9, AAV10, or a chimeric AAV antigen. In certain embodiments, the AAV antigen is an AAV8 antigen. In certain embodiments, the AAV antigen is from a recombinant AAV (rAAV). In certain embodiments, the AAV antigen is from a genetically engineered AAV, or chemically modified AAV, or both.

In certain embodiments, the ELISA comprises an antibody specific for an AAV epitope. In certain embodiments, the AAV epitope is a conformational epitope present on assembled AAV capsids. In certain embodiments, the AAV epitope is a linear epitope present on assembled AAV capsids. Examples of such epitopes includes, but is not limited to, capsid virion proteins VP1, VP2, and/or VP3. In certain embodiments, the AAVs are genetically engineered to express additional virion proteins on the surface of the capsid, and those engineered proteins can be used/detected in an ELISA assay. In certain embodiments, the AAV are chemically modified to express variants of virion protein, which can be used/detected in an ELISA assay (e.g., VP1′, VP2′, VP3′, etc. . . . ). In certain embodiments, the antibodies identify serotype specific capsid virion proteins.

The ELISA may replace qPCR as a way to determine the dose and/or potency of an AAV fraction, preparation, product, formulation, or composition. The ELISA technique of the invention has significantly less variability than the qPCR method. In certain embodiments, the methods of the present disclosure comprise evaluating an AAV fraction, preparation, product, formulation, or composition via an AAV-specific ELISA. In certain embodiments, all the methods disclosed herein do not include a qPCR step.

In certain embodiments, the methods of the present disclosure comprise testing an AAV fraction or preparation obtained after ultracentrifugation via an AAV-specific ELISA to determine the number of AAV capsids in the AAV fraction or preparation. In certain embodiments, the methods of the present disclosure comprise testing an AAV fraction or preparation obtained after depth filtration via an AAV-specific ELISA to determine the number of AAV capsids in the AAV fraction or preparation. In certain embodiments, the methods of the present disclosure comprise testing an AAV fraction or preparation obtained after concentrating an AAV fraction or preparation using an ultra-/diafiltration system via an AAV-specific ELISA to determine the number of AAV capsids in the AAV fraction or preparation. In certain embodiments, the methods of the present disclosure comprise testing an AAV fraction or preparation obtained after a tangential flow filtration (TFF) step via an AAV-specific ELISA to determine the number of AAV capsids in the AAV fraction or preparation. In certain embodiments, the methods of the present disclosure comprise testing an AAV fraction or preparation obtained after negative anion exchange (AEX) chromatography via an AAV-specific ELISA to determine the number of AAV capsids in the AAV fraction or preparation. In certain embodiments, the methods of the present disclosure comprise testing an AAV fraction or preparation obtained after a polish step via an AAV-specific ELISA to determine the number of AAV capsids in the AAV fraction or preparation. In certain embodiments, the methods of the present disclosure comprise testing a purified AAV fraction or preparation via an AAV-specific ELISA to determine the number of AAV capsids in the AAV fraction or preparation.

Additional methods for quantitating the number of AAV capsids includes, but are not limited to, surface plasmon resonance (SPR) (e.g. BIACORE, OCTET), differential scanning fluorimetry (e.g., Prometheus NT48, Nanotemper), magnetic immuno assay (MIA), and cloned enzyme donor immunoassay (CEDIA). These methods can be used in addition to or in place of the ELISA quantitation assay.

In certain embodiments, the methods of the present disclosure comprise measuring full versus empty AAV capsids (e.g., percentage of ratio of full versus empty AAV capsids). Method for evaluating or confirming the percentage or ratio of full:empty AAV capsids in an AAV fraction or preparation include, but are not limited to, cryogenic transmission electron microscopy (CryoTEM), negative staining TEM, capillary electrophoresis, analytical ultracentrifugation, or combinations thereof.

In certain embodiments, the method for measuring full versus empty AAV capsids is CryoTEM. In certain embodiments, the method entails using both CryoTEM and an AAV-specific ELISA as described above. In certain embodiments, the ELISA is a sandwich ELISA. In certain embodiments, the sandwich ELISA comprises an antibody specific for an AAV epitope. In certain embodiments, the AAV epitope is a conformational epitope present on assembled AAV capsids.

One advantage of using CryoTEM is that there is no need for a negative stain. CryoTEM also results in the ability to quantitate the amount of full AAV capsids. In certain embodiments, use of CryoTEM results in not overestimating the full AAV capsid amount due to a false positive signal.

The methods of the present disclosure comprise a method evaluating the number or a percentage of full versus empty AAV capsids. In certain embodiments, the methods comprise CryoTEM. In certain embodiments, the methods comprise: (i) embedding an AAV fraction or preparation in a substrate in an inert support; (ii) flash-freezing the embedded AAV fraction or preparation; (iii) imaging the embedded AAV fraction or preparation using cryogenic transmission electron microscopy; and (iv) quantitating the percentage of full AAV capsids versus empty AAV capsids.

For step (i), examples of suitable substrates for use in the method includes, but is not limited to amorphous, non-crystalline ice. Examples of suitable inert support for use in the method includes, but is not limited to a film of carbon, thermoplastic resins, and polyvinyl formals (e.g., polymers formed from polyvinyl alcohol and formaldehyde as copolymers with polyvinyl acetate, such as, but not limited to, Formvar or Vinylec, polyvinyl formal stabilized with carbon, silicon monoxide on polyvinyl formal, pure carbon film, carbon type-A: carbon support films (e.g., removable polyvinyl formal on the opposite side of the grid), carbon type-B: polyvinyl formal film (e.g., coated with a heavier layer of carbon), and silicon monoxide on carbon type-A). In certain embodiments, step (i) above involves deposition of a sample onto a thin supporting film of carbon in a temperature (e.g., (−196° C.) or below) and humidity controlled environment. In certain embodiments, the humidity level can be relative humidity. For step (ii), a sample can be flash-frozen. In certain embodiments, flash freezing can entail using liquid ethane, liquid nitrogen, liquid propane, or helium near liquid nitrogen temperature. For example, the container of liquid ethane, liquid nitrogen, liquid propane, or helium is surrounded by liquid nitrogen.

In certain embodiments, the AAV fraction or preparation is frozen so rapidly (e.g., at 104 to 106 K per second) that ice crystals are unable to form. In certain embodiments, amorphous ice is produced either by rapid cooling of liquid water or by compressing ordinary ice at low temperatures. In certain embodiments, after excess of AAV fraction or preparation is removed leaving some of the specimen adhered, the grid is vitrified in liquid ethane and then stored in liquid nitrogen. For discussion of additional steps to perform the CryoTEM, see Cabra and Samso, J. Visualized Experiments, (2015) 95(e52311):1-11, incorporated by reference herein in its entirety for all purposes.

CryoTEM analysis of AAV particles can be utilized to assess the overall specimen morphology, i.e. presence of various AAV morphologies (generally including spherical and deformed AAV particles, subunit structures and larger structurally less defined morphologies). For example, the full capsid displays an inner density with no distinct boundary between the shell and the core. AAV capsids displaying a distinct outer shell and minute internal density are classified as empty capsids.

CryoTEM can also be used to assess uncertain capsids. For example, CryoTEM can be used to count “full”, “empty”, and “uncertain” capsids.

CryoTEM can also be used to determine the level of packaging of the particles by either manually classifying particles or using an automated image analysis methodology.

In certain embodiments, the methods of the present disclosure comprise testing an AAV fraction or preparation obtained after ultracentrifugation via CryoTEM to determine the quantity and/or quality of the AAV capsids in the AAV fraction or preparation. In certain embodiments, the methods of the present disclosure comprise testing an AAV fraction or preparation obtained after depth filtration via CryoTEM to determine the quantity and/or quality of the AAV capsids in the AAV fraction or preparation. In certain embodiments, the methods of the present disclosure comprise testing an AAV fraction or preparation obtained after concentrating an AAV fraction or preparation using an ultra-/diafiltration system via CryoTEM to determine the quantity and/or quality of the AAV capsids in the AAV fraction or preparation. In certain embodiments, the methods of the present disclosure comprise testing an AAV fraction or preparation obtained after a tangential flow filtration (TFF) step via CryoTEM to determine the quantity and/or quality of the AAV capsids in the AAV fraction or preparation. In certain embodiments, the methods of the present disclosure comprise testing an AAV fraction or preparation obtained after negative anion exchange (AEX) chromatography via CryoTEM to determine the quantity and/or quality of the AAV capsids in the AAV fraction or preparation. In certain embodiments, the methods of the present disclosure comprise testing an AAV fraction or preparation obtained after a polishing step via CryoTEM to determine the quantity and/or quality of the AAV capsids in the AAV fraction or preparation. In certain embodiments, the methods of the present disclosure comprise testing a purified AAV fraction or preparation via CryoTEM to determine the quantity and/or quality of the AAV capsids in the AAV fraction or preparation.

In certain embodiments, the methods of the present disclosure comprise testing an AAV fraction or preparation obtained after ultracentrifugation via an AAV-specific ELISA and CryoTEM to determine the quantity and quality of the AAV capsids in the AAV fraction or preparation. In certain embodiments, the methods of the present disclosure comprise testing an AAV fraction or preparation obtained after depth filtration via an AAV-specific ELISA and CryoTEM to determine the quantity and quality of the AAV capsids in the AAV fraction or preparation. In certain embodiments, the methods of the present disclosure comprise testing an AAV fraction or preparation obtained after concentrating an AAV fraction or preparation using an ultra-/diafiltration system via an AAV-specific ELISA and CryoTEM to determine the quantity and quality of the AAV capsids in the AAV fraction or preparation. In certain embodiments, the methods of the present disclosure comprise testing an AAV fraction or preparation obtained after a tangential flow filtration (TFF) step via an AAV-specific ELISA and CryoTEM to determine the quantity and quality of the AAV capsids in the AAV fraction or preparation. In certain embodiments, the methods of the present disclosure comprise testing an AAV fraction or preparation obtained after negative anion exchange (AEX) chromatography via an AAV-specific ELISA and CryoTEM to determine the quantity and quality of the AAV capsids in the AAV fraction or preparation. In certain embodiments, the methods of the present disclosure comprise testing an AAV fraction or preparation obtained after a polishing step via an AAV-specific ELISA and CryoTEM to determine the quantity and quality of the AAV capsids in the AAV fraction or preparation. In certain embodiments, the methods of the present disclosure comprise testing a purified AAV fraction or preparation via an AAV-specific ELISA and CryoTEM to determine the quantity and quality of the AAV capsids in the AAV fraction or preparation.

Analytical Ultracentrifugation (AUC) is capable of separating proteins according to the sedimentation coefficient. For identical proteins, the sedimentation coefficient can be correlated to aggregation status. Briefly, samples are diluted with the corresponding sample buffer and then transferred to cell assemblies with built-in quartz windows and loaded in the rotor, which is then rotated at a constant speed. Protein molecules of different size migrate at different sedimentation speed towards the bottom of the cell and are monitored continuously during centrifugation by UV detection at 280 nm. The collected data set allows for a computational analysis, which deconvolutes the sedimentation and diffusion processes, resulting in a differential sedimentation coefficient distribution c(s). This resolves the different species of the sample, and presents their s-values and populations. The distribution of sedimentation coefficients is integrated and relative area percentages as well as S-values of the peak maxima are given as results of the analysis.

### AAV Preparation

An AAV preparation produced by a method of the present disclosures is further provided herein. AAV preparation and AAV fraction are used interchangeably for the purposes of this disclosure. In certain embodiments, an AAV fraction is an AAV preparation that has been further concentrated or a portion of the AAV removed from the AAV preparation. In certain embodiments, the method for producing an AAV preparation, comprising: (i) transfecting host cells with at least one plasmid comprising the gene of interest; (ii) collecting supernatant or cell suspension of a cell culture comprising AAV capsids to create an AAV fraction or preparation; (iii) quantifying the total number of AAV capsids in the AAV fraction or preparation using an AAV-specific ELISA assay; and (iv) preparing an AAV product, formulation, or composition with a desired concentration based on the total number of AAV capsids determined in step (iii). In certain embodiments, the method comprises concentrating the AAV fraction or preparation. In certain embodiments, the method comprises removing at least a portion of empty capsids from the AAV fraction or preparation. In certain embodiments, the amount of empty capsids are removed to create an AAV fraction, preparation, product, AAV formulation or AAV composition with a specific concentration of full capsids and/or a specific ratio of full:empty capsids. In certain embodiment, the AAV fraction or preparation is diluted with the appropriate buffer to the desired dose.

In certain embodiments, the method comprises evaluating or confirming the percentage or ratio of full versus empty (full:empty) AAV capsids in the AAV fraction, preparation, product, formulation, or composition. In certain embodiments, the method comprises evaluating or confirming the percentage or ratio of full versus empty (full:empty) AAV capsids is CryoTEM. In certain embodiments, the dose and/or potency is not determined by qPCR. In certain embodiments, the percentage of full AAV capsids is about 40% to about 100%. In certain embodiment, the percentage of full AAV capsids is from about 40% to about 95%, about 40% to about 90%, about 40% to about 85%, about 40% to about 80%, about 45% to about 75%, about 50% to about 70%, or about 55% to about 65%. In certain embodiments, the percentage of full AAV capsids is between about 60% to about 80%. In certain embodiments, at least about 60% of the AAV capsids are full AAV capsids. In certain embodiments, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% of the AAV capsids are full AAV capsids. In certain embodiments, the methods as disclosed herein are used to generate AAV fractions, preparations, product, formulation, or composition with consistent amounts of full capsids between AAV fractions, preparation, product, formulation, or composition.

In certain embodiments, the method comprises evaluating or confirming the percentage or ratio of full versus empty (full:empty) AAV capsids in the AAV fraction, preparation, product, formulation, or composition. In certain embodiments, the method comprises evaluating or confirming the percentage or ratio of full versus empty (full:empty) AAV capsids is CryoTEM. In certain embodiments, the dose and/or potency is not determined by qPCR. In certain embodiments, the percentage of full AAV capsids is about 40% to about 100%. In certain embodiment, the percentage of full AAV capsids is from about 40% to about 95%, about 40% to about 90%, about 40% to about 85%, about 40% to about 80%, about 45% to about 75%, about 50% to about 70%, or about 55% to about 65%. In certain embodiments, the percentage of full AAV capsids is between about 60% to about 80%. In certain embodiments, at least about 60% of the AAV capsids are full AAV capsids. In certain embodiments, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% of the AAV capsids are full AAV capsids. In certain embodiments, the methods as disclosed herein are used to generate AAV fractions, preparations, product, formulation, or composition with consistent amounts of full capsids between AAV fractions or preparation.

A method of administering an AAV product, formulation, or composition produced by a method of the present disclosures is further provided herein. In certain aspects, methods of the invention entail administering an AAV product, formulation, or composition of a specific dose to a subject in need thereof, comprising (i) obtaining a purified AAV preparation; (ii) measuring the concentration of AAV capsids in the purified AAV preparation using an AAV-specific ELISA assay; (iii) administering a specific dose of the AAV product, formulation, or composition to the subject. In certain embodiments, the preparation in steps (i) and (ii) is an AAV fraction or preparation that can be used to generate a final AAV product, formulation, or composition, in which the AAV fraction or preparation is further purified or diluted to form the AAV product, formulation, or composition for steps (i), (ii), and/or (iii). In certain embodiments, the method comprises evaluating or confirming the percentage or ratio of full versus empty (full:empty) AAV capsids in the AAV preparation, product, formulation, or composition. In certain embodiments, the method comprises evaluating or confirming the percentage or ratio of full versus empty (full:empty) AAV capsids is CryoTEM. In certain embodiments, the concentration, dose, and/or potency is not determined by qPCR. In certain embodiments, the percentage of full AAV capsids is about 40% to about 100%. In certain embodiment, the percentage of full AAV capsids is from about 40% to about 95%, about 40% to about 90%, about 40% to about 85%, about 40% to about 80%, about 45% to about 75%, about 50% to about 70%, or about 55% to about 65%. In certain embodiments, the percentage of full AAV capsids is between about 60% to about 80%. In certain embodiments, at least about 60% of the AAV capsids are full AAV capsids. In certain embodiments, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% of the AAV capsids are full AAV capsids.

In certain embodiments, the potency of an AAV preparation, product, formulation, or composition can be further verified by an in vitro and/or in vivo biopotency assay. For example, the steps can entail, 1) determining the content of AAV in the preparation (e.g., via ELISA); 2) determining the percentage of full capsids (e.g., via CryoTEM) and 3) confirming biological activity (e.g., via an in vivo and/or in vitro biopotency assay).

In certain embodiments, for the methods, fractions, preparations, products, formulations, or compositions disclosed herein, the concentration of the AAV preparations is between about 1×10¹⁰ cp/ml to about 1×10²⁰ cp/ml. In certain embodiments, for the methods, fractions, preparations, products, formulations, or compositions disclosed herein, the concentration of the AAV preparations, products, formulations, or compositions is between about 1×10¹¹ cp/ml to about 1×10¹⁹ cp/ml, about 1×10¹² cp/ml to about 1×10¹⁸ cp/ml, about 1×10¹³ cp/ml to about 1×10¹⁷ cp/ml, or about 1×10¹⁴ cp/ml to about 1×10¹⁶ cp/ml. In certain embodiments, for the methods and preparations, products, formulations, or compositions disclosed herein, the concentration of the AAV preparation, products, formulations, or compositions is between about 1×10¹² cp/ml to about 1×10¹⁵ cp/ml, about 1×10¹³ cp/ml to about 1×10¹⁵ cp/ml, or about 1×10¹² cp/ml to about 1×10¹³ cp/ml. In certain embodiments, for the methods and preparations, products, formulations, or compositions disclosed herein, the concentration of the AAV preparation, product, formulation, or composition is between about 1×10¹⁴ cp/ml to about 5×10¹⁴ cp/ml, about 2×10¹⁴ cp/ml to about 3×10¹⁴ cp/ml, or about 3.5×10¹⁴ cp/ml to about 5×10¹⁵ cp/ml. In certain embodiments, cp/ml can be total capsids per ml or full capsids per ml. In certain embodiments, the cp/ml is total capsids per ml.

In certain embodiments, the AAV preparation, product, formulation, or composition comprises at least about 10¹² virus particles (vp) produced from about 1000 L of starting material (e.g., cell culture) or at least about 10¹³ virus particles (vp) produced from about 1000 L of starting material (e.g., cell culture).

In certain embodiments, for the methods or preparations, products, formulations, or compositions disclosed herein, the dose of the AAV preparation, product, formulation, or composition is between about 1×10⁵ cp/kg to about 1×10²⁵ cp/kg. In certain embodiments, for the methods or preparations, products, formulations, or compositions disclosed herein, the dose of the AAV preparation, product, formulation, or composition is between about 1×10⁶ cp/kg to about 1×10²⁴ cp/kg, about 1×10⁷ cp/kg to about 1×10²³ cp/kg, about 1×10⁸ cp/kg to about 1×10²² cp/kg, about 1×10⁹ cp/kg to about 1×10²¹ cp/kg, about 1×10¹⁰ cp/kg to about 1×10²⁰ cp/kg, about 1×10¹¹ cp/kg to about 1×10¹⁹ cp/kg, about 1×10¹² cp/kg to about 1×10¹⁸ cp/kg, about 1×10¹³ cp/kg to about 1×10¹⁷ cp/kg, or about 1×10¹⁴ cp/kg to about 1×10¹⁶ cp/kg. In certain embodiments, for the methods and preparations, products, formulations, or compositions disclosed herein, the dose of the AAV preparation, product, formulation, or composition is between about 1×10¹² cp/kg to about 1×10²⁰ cp/kg, about 1×10¹³ cp/kg to about 1×10¹⁹ cp/kg, about 1×10¹⁴ cp/kg to about 1×10¹⁸ cp/kg, or about 1×10¹⁵ cp/kg to about 1×10¹⁷ cp/kg. In certain embodiments, for the methods and preparations, products, formulations, or compositions disclosed herein, the dose of the AAV preparation, product, formulation, or composition is between about 1×10¹⁰ cp/kg to about 1×10¹⁶ cp/kg. In certain embodiments, for the methods and preparations, products, formulations, or compositions disclosed herein, the dose of the AAV preparation, product, formulation, or composition is at least about 1×10⁶ cp/kg, at least about 1×10⁷ cp/kg, at least about 1×10⁸ cp/kg, at least about 1×10⁹ cp/kg, at least about 1×10¹⁰ cp/kg, at least about 1×10¹¹ cp/kg, at least about 1×10¹² cp/kg, at least about 1×10¹³ cp/kg, at least about 1×10¹⁴ cp/kg, at least about 1×10¹⁵ cp/kg, at least about 1×10¹⁶ cp/kg, at least about 1×10¹⁷ cp/kg, at least about 1×10¹⁸ cp/kg, at least about 1×10¹⁹ cp/kg, at least about 1×10²⁰ cp/kg, at least about 1×10²¹ cp/kg, at least about 1×10²² cp/kg, at least about 1×10²³ cp/kg, at least about 1×10²⁴ cp/kg, or at least about 1×10²⁵ cp/kg. In certain embodiments, cp/kg can be total capsids per kg of the subject or full capsids per kg of the subject. In certain embodiments, the cp/kg is total capsids per kg of the subject.

In certain embodiments, the AAV preparation, product, formulation, or composition of the present disclosures is highly pure, highly potent and suitable for clinical use in a subject. In certain embodiments, the AAV preparation, product, formulation, or composition comprises AAV capsid particles of a homogenous population and high purity. In certain embodiments, the AAV preparation, product, formulation, or composition comprises full-length vector DNA. In exemplary embodiments, the AAV preparation, product, formulation, or composition is substantially free of unwanted contaminants, including but not limited to, AAV capsid particles containing truncated or incomplete vector DNA, AAV particles with incomplete protein composition and oligomerized structures, or contaminating viruses, e.g., non AAV, lipid enveloped viruses. In exemplary embodiments, the AAV preparation, product, formulation, or composition contains a high amount of encoding cDNA of the protein of interest. In certain embodiments, the AAV preparation, product, formulation, or composition of the present disclosure is suitable for administration to a subject. In certain embodiments, the AAV preparation, product, formulation, or composition is sterile and/or of good manufacturing practice (GMP) grade. In certain embodiments, the AAV preparation, product, formulation, or composition conforms to the requirements set forth in the U.S. Pharmacopeia Chapter 1046 or the European Pharmacopoeia on gene therapy medicinal products or as mandated by the U.S. Food and Drug Administration (USFDA) or the European Medicines Agency (EMA). In certain embodiments, the AAV preparation, product, formulation, or composition is a ready-to-use preparation, product, formulation, or composition for direct administration to a subject with little to no processing or handling.

### Source of AAV

With regard to the methods of the present disclosure, the AAV may be of any AAV serotype. In certain embodiments, the AAV described herein are of AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, or chimeric AAV vectors. In certain embodiments, the AAV is wild type. In certain embodiments, the AAV is a recombinant AAV (rAAV). In certain embodiments, the AAV is modified by genetic engineering and/or is chemically modified. In certain embodiments, the AAV comprises a modified capsid, e.g., a genetically engineered or a chemically-modified AAV capsid. In certain embodiments, the AAV is of the AAV8 serotype. In certain embodiments, the AAV is of the AAV9 serotype.

With regard to the methods of the invention, the AAV fraction or preparation is in exemplary aspects a concentrated AAV fraction or preparation. In certain embodiments, the AAV fraction or preparation comprises at least about 1×10¹⁰, about 1×10¹¹, about 1×10¹², about 1×10¹³, about 1×10¹⁴, about 1×10¹⁵, or about 1×10¹⁶, AAV total capsids per mL. In certain embodiments, the AAV fraction or preparation comprises at least about 1×10¹² AAV total capsids per mL. The AAV capsids may include empty AAV capsids and full AAV capsids.

In certain embodiments, the AAV represents an AAV fraction or preparation produced by transfected host cells. In certain embodiments, the AAV fraction or preparation represents a supernatant harvested or cell suspension from a cell culture comprising host cells transfected with a triple plasmid system, wherein one plasmid of the system comprises a gene or cDNA of interest, one plasmid encodes capsid protein VP1, capsid protein VP2 and/or capsid protein VP3. In certain embodiments, VP1, VP2, and/or VP3 are AAV8 VP1, VP2, and/or VP3. Triple plasmid transfection for purposes of rAAV production is known in the art. See, e.g., Qu et al., 2015, supra, and Mizukami et al., “A Protocol for AAV vector production and purification.” PhD dissertation, Division of Genetic Therapeutics, Center for Molecular Medicine, 1998; and Kotin et al., Hum Mol Genet 20(R1): R2-R6 (2011). In certain embodiments, the transfection may be carried out using inorganic compounds, e.g., calcium phosphate, or organic compounds, polyethyleneimine (PEI), or non-chemical means, e.g., electroporation.

In certain embodiments, the host cells are adherent cells. In certain embodiments, the host cells are suspension cells. In certain embodiments, the host cells are HEK293 cells or Sf9 cells (e.g., baculovirus infected Sf9 cells) or HeLa or BHK (Herpes Virus System). In certain embodiments, the cell culture comprises culture medium which is serum and protein free. In certain embodiments, the medium is chemically defined and is free of animal derived components, e.g., hydrolysates.

In certain embodiments, the fraction or preparation comprising rAAV particles represents a fraction or preparation comprising HEK293 cells transfected with a triple plasmid system. In certain embodiments, the fraction or preparation comprising AAV particles represents a fraction or preparation of the harvest after about 2 to about 7 days after transfection of the HEK293 cells or when the cell culture has a cell density of greater than or about 5×10⁶ cells/mL and has a cell viability of greater than or about 50%.

In certain embodiments, the AAV is prepared by a triple plasmid transfection followed by harvest from one to 7 days later. In certain embodiments, the AAV is prepared from cell disruption.

In certain embodiments, the AAV is prepared by the following: The HEK293 cells are adherent and grown in a commercially-available culture medium that may be chemically-defined and may be free of animal-derived components, e.g. serum and proteins. The cells are cultured to a cell density of about 3×10⁶ to about 12×10⁶ cells/ml, e.g., about 6×10⁶ to about 10×10⁶ cells/ml. The cells are then split in about a 1:2 ratio such that the cell density is about 3-5×10⁶ cells/ml. After the split, the cells may be transfected with three plasmids that include (1) a helper plasmid capable of providing one or more helper viral functions essential AAV production, (2) a plasmid that encodes for one or more genes involved in capsid generation, replication and packaging of the virus, and (3) a plasmid comprising a gene of interest (GOI) to be packaged into the resulting rAAV particle. For example, the GOI may be a vector DNA comprising human coagulation Factor IX Padua in a single stranded self-complementary form, with the vector DNA. As another example, the GOI may be a vector DNA comprising human coagulation Factor IX Padua in a double stranded self-complementary form, with the vector DNA having a full length of 4.8 kB. As another example, the GOI may be a vector DNA comprising a B-domain deleted human coagulation Factor VIII in a single stranded self-complementary form, with the vector DNA having a full length of 4.8 kB. Other GOI may be used. Transfection may be carried out in a transient manner, such as by using cationic polymers. Before elution, the HEK293 cell line may be cultivated for at least about 1 days, e.g., 3-5 days, before harvesting.

### Example Section

### Example 1: General Purification Procedures

The following examples are given merely to illustrate the present invention and not in any way to limit its scope.

AAV8 production was developed in a HEK293 cell line after transfection with a triple plasmid system containing encoding cDNA of the protein of interest and AAV8-. VP1.-VP2 and -VP3. The clarified cell free culture supernatant was concentrated and diafiltrated with Pall Omega T-Series Cassette 100 kDa. The viral particles were loaded onto a membrane adsorber (MustangQ. Pall Part Number XT140MSTGQP05) at nonbinding conditions. The obtained AAV8 containing flow through was used as Load for the following affinity purification step.

A column containing POROS™ CaptureSelect™ AAV8 Affinity Matrix (Cat. No. A30794. Thermo Fisher) ID 32 mm, with a bed height of 60 mm and a volume approx. 200 ml, was equilibrated with at least five column volumes of 50 mM TrisHCl and 125 mM NaCl at pH 8.5. The LOAD was applied onto the column containing POROS™ CaptureSelect™ AAV8 Affinity Matrix (Cat. No. A30794. Thermo Fisher). The column was then re-equilibrated with 5 column volumes of 50 mM TrisHCl and 125 mM NaCl at pH 8.5. The column was then washed with 5 column volumes of Wash 1 (W1): 100 mM Sodium Acetate and 0.1% Tween80 at pH 6.0. The column was then washed with 5 column volumes of Wash 2 (W2): 50 mM TrisHCl and 125 mM NaCl at pH 8.5. The column was then washed with 5 column volumes of Wash 3 (W3): 50 mM TrisHCl and 50% ethylene glycol at pH 8.5.

Elution was undertaken by applying 5 column volumes of the following elution buffer to the column: 50 mM TrisHCl, 50% ethylene glycol and 750 mM NaCl, at pH 8.0.

The above procedure is summarized in Table 1.

TABLE 1
General purification scheme for AAV8-Affinity

| Step | High pH LOAD | CV | Flowrate |
|---|---|---|---|
| 1. | 50 mM TrisHCl | ≥5 | 60 cm/h |
| | 125 mM NaCl | | |
| | pH 8.5 | | |
| 2. | Sample-Load | | |
| | pH 8.5 | | |
| 3. | 50 mM TrisHCl | 5 | |
| | 125 mM NaCl | | |
| | pH 8.5 | | |
| 4. | 100 mM NaAcetat | 5 | |
| | 0.1% Tween80 | | |
| | pH 6.0 | | |
| 5. | 50 mM TrisHCl | 5 | |
| | 125 mM NaCl | | |
| | pH 8.5 | | |
| 6. | 50 mM TrisHCl | 5 | 30 cm/h |
| | 50% Ethylenglycol | | |
| | pH 8.5 | | |
| 7. | ELUTION | 5 | |
| | 50 mM TrisHCl | | |
| | 50% Ethylene glycol | | |
| | 750 mM NaCl | | |
| | pH 8.0 | | |
| 8. | Post-Elution | 5 | |
| | 50 mM TrisHCl | | |
| | 50% Ethylene glycol | | |
| | 2000 mM NaCl | | |
| | pH 8.0 | | |

A buffer exchange of the eluate into 30 mM NaAcetate, 2 mM CaAcetate, 0.005% Polysorbate 80, pH 6.0 was performed with VIVACELL100 (10K) cartridges (Sartorius) to provide proper binding properties for all AAV-Subtypes on cation exchanger (CEX).

### Example 2: AAV8, Vector Genome Size 2.6 kB, Capto S, in Presence of Calcium

The following test procedure was undertaken. Note that all buffers disclosed in this example were made at room temperature and the pH of all buffers were measured at room temperature. First, a column containing Capto S Cation Exchanger Resin (Cat. 17-5441-01; Cytiva) ID 11 mm, with a bed height of 100 mm, an area of 0.95 cm², and a volume of approximately 9.5 ml, was equilibrated (activation) with 5 column volumes of a buffer comprising 1000 mM Sodium acetate, 2 mM Calcium acetate, 0.005% Polysorbate 80 at a pH of 6.0. The column was then equilibrated with at least five column volumes of 30 mM Sodium acetate, 2 mM Calcium acetate, 0.005% Polysorbate 80 at a pH of 6.0. The LOAD (AAV conditioned in 30 mM Sodium acetate, 2 mM Calcium acetate, 0.005% Polysorbate 80 at a pH of 6.0) was applied onto the column containing Capto S Cation Exchanger Resin.

The column was then washed with five column volumes of 30 mM Sodium acetate, 2 mM Calcium acetate, 0.005% Polysorbate 80 at a pH of 6.0.

For the elution a Gradient elution was then performed. The Gradient was performed with 40 column volumes of 30 mM Sodium acetate, 2 mM Calcium acetate, 0.005% Polysorbate 80 at a pH of 6.0 to 200 mM Sodium acetate, 2 mM Calcium acetate, 0.005% Polysorbate 80 at a pH of 6.0. Post elution was performed with 10 column volumes of 1000 mM Sodium acetate, 2 mM Calcium acetate, 0.005% Polysorbate 80 at a pH of 6.0.

For all steps the linear flow rate was 60 cm/h.

Buffer compositions are summarized in Table 2.

TABLE 2
Buffer composition for CEX-Separation

| Buffer | Buffer content | pH |
|---|---|---|
| TWA (2M NaCl) | 2000 mmol NaCl | n.d |
| AC1 | 30 mM NaAcetate | pH 6.0 ± 0.2 |
| | 2 mM CaAcetate | |
| | 0.005% Polysorbate 80 | |
| ACE | 200 mM NaAcetate | pH 6.0 ± 0.2 |
| | 2 mM CaAcetate | |
| | 0.005% Polysorbate 80 | |
| ACNE | 1000 mM NaAcetate | pH 6.0 ± 0.2 |
| | 2 mM CaAcetate | |
| | 0.005% Polysorbate 80 | |

Table 3 shows the chromatographic scheme for CEX-Separation

TABLE 3

| Step | | Buffer | Amount CV | Flow rate cm/h |
|---|---|---|---|---|
| 1 | Equilibration 1 | TWA (2M NaCl) | 5 | 60 |
| 2 | Equilibration 2 | AC1 | 5 | 60 |
| 3 | Product load | diluted affinity | X | 60 |
| | | Eluate | | |
| 4 | Wash 1 | AC1 | 5 | 60 |
| 5 | Elution | Gradient 0-100% | 40 | |
| | | ACE | | 60 |
| | | in AC1 | | |
| | Collecting | | 4 ml Fractions | |
| 7 | Post elution | ACNE | 10 | 60 |

Table 4 shows the ratio of ddPCR/AAV8:AG

TABLE 4

| | Ratio |
|---|---|
| | ddPCR/AAV8:AG |
| | vg/cp |
| LOAD | 0.26 |
| E1 | 0.20 |
| E2 | 1.64 |
| E3 | 1.73 |
| E4 | 0.27 |
| E5 | 0.14 |
| E6 | 0.16 |
| E7 | 0.10 |
| E8 | 0.14 |
| E9 | 0.13 |

ddPCR: FIX-specific ddPCR/AAV8:AG determined with AAV8 Antigen ELISA

Table 5 shows the percentage of full and empty capsids determined with AUC.

TABLE 5

| | Area % | Area % | Area % | Area % | S | S | S | S |
|---|---|---|---|---|---|---|---|---|
| Name | empty | subpopulation | Full | Aggregates | empty | subpopulation | Full | Aggregates |
| LOAD | 36.4 | 0.8 | 56.6 | 6.2 | 64.9 | 74.3 | 88.8 | 98.2 |
| E2 | 5.1 | 0.0 | 91.7 | 3.1 | 65.8 | 76.9 | 88.0 | 118.7 |
| E3 | 17.0 | 0.2 | 72.2 | 10.7 | 64.1 | 78.6 | 88.0 | 101.7 |
| E4 | 39.0 | 4.9 | 50.4 | 5.7 | 64.1 | 74.3 | 88.0 | 104.2 |
| E5 | 44.4 | 2.1 | 50.6 | 2.9 | 63.2 | 75.2 | 87.1 | 111.1 |

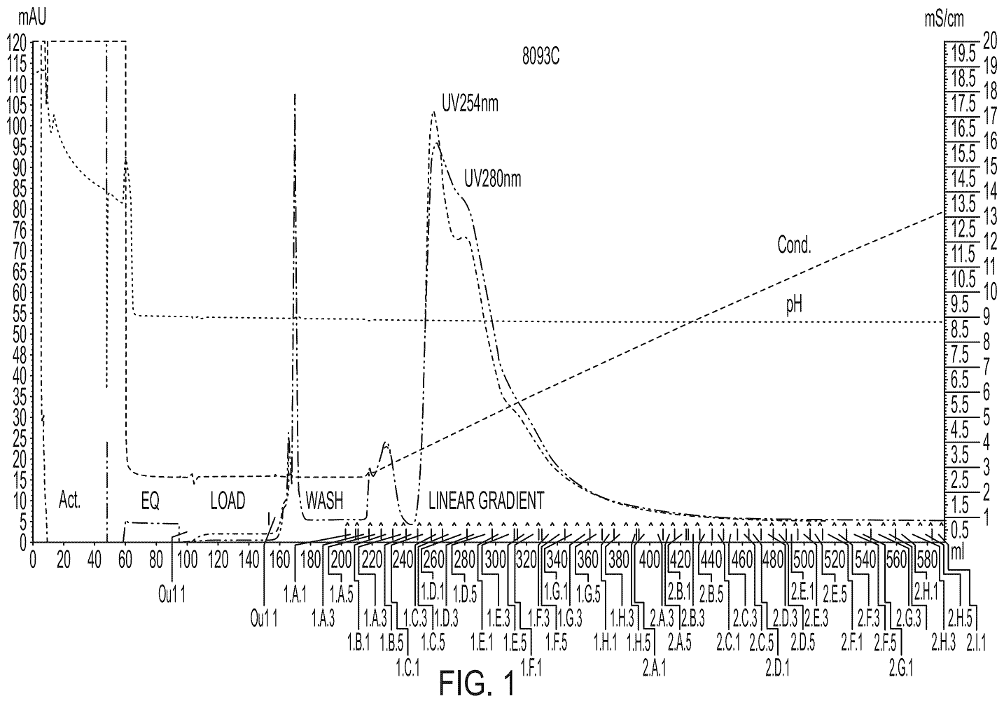

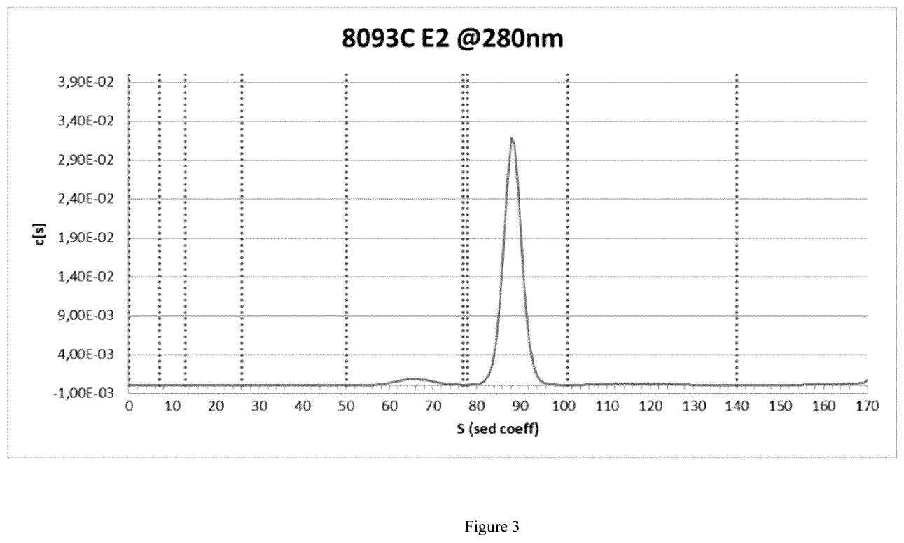

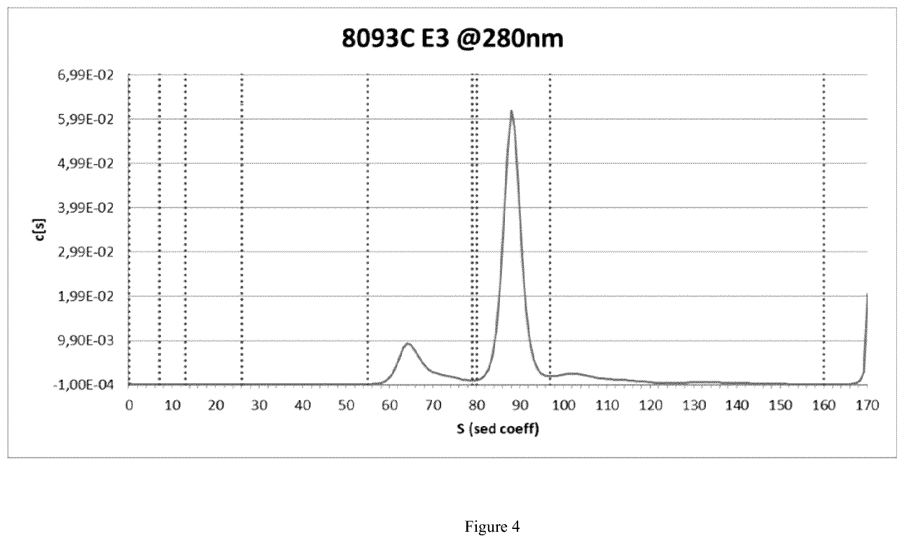

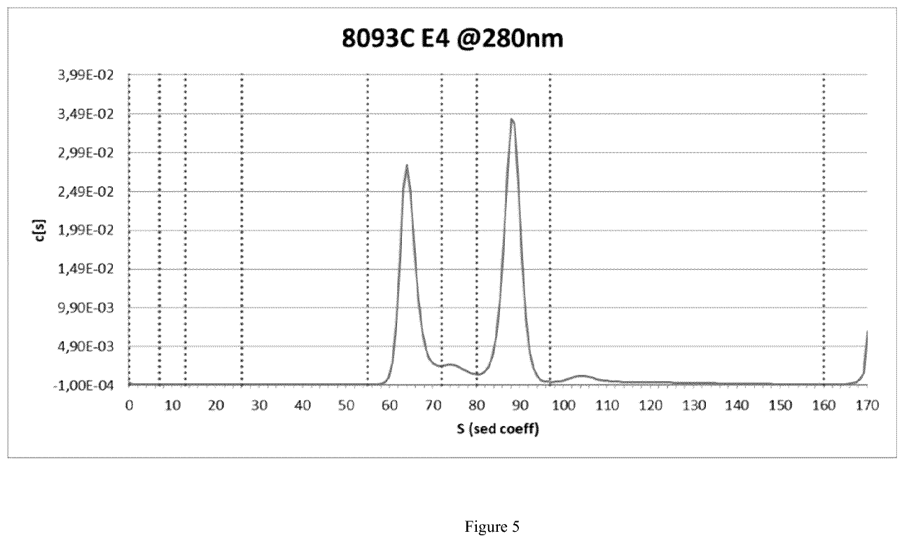

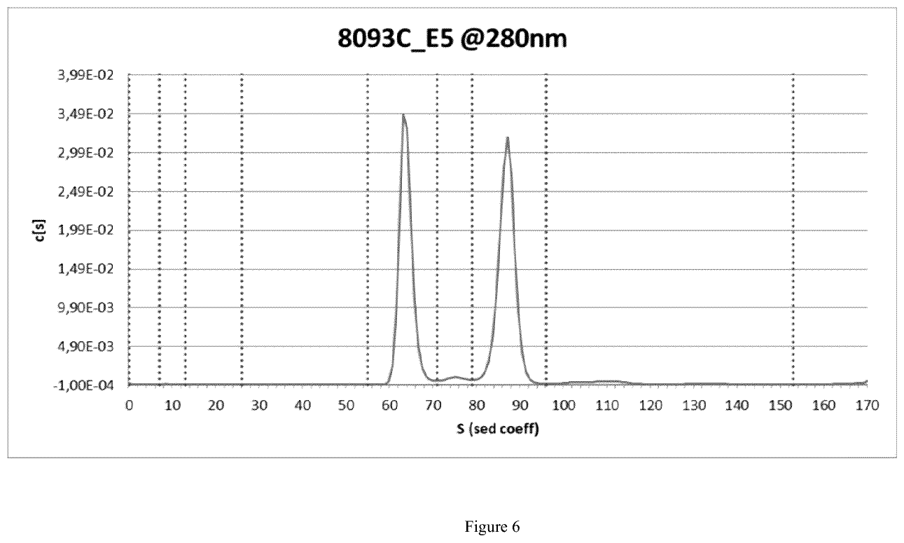

Results are shown in FIGS. 1-6 and yields are shown in Table 6.

FIGS. 1-6 represents a run with data of most of the fractions. LOAD, FT=Flow through, W=Wash, E=Eluate, ET-E9=Pool—fractions of the peak according to the chromatogram. X-Axis: UV28 nm(left) conductivity (right), Y-Axis: Volume (ml). The ratio vg/cp which indicates the AAV's with the highest amount of full capsids (Full capsid fractions). The higher the value the higher is the amount of full capsids.

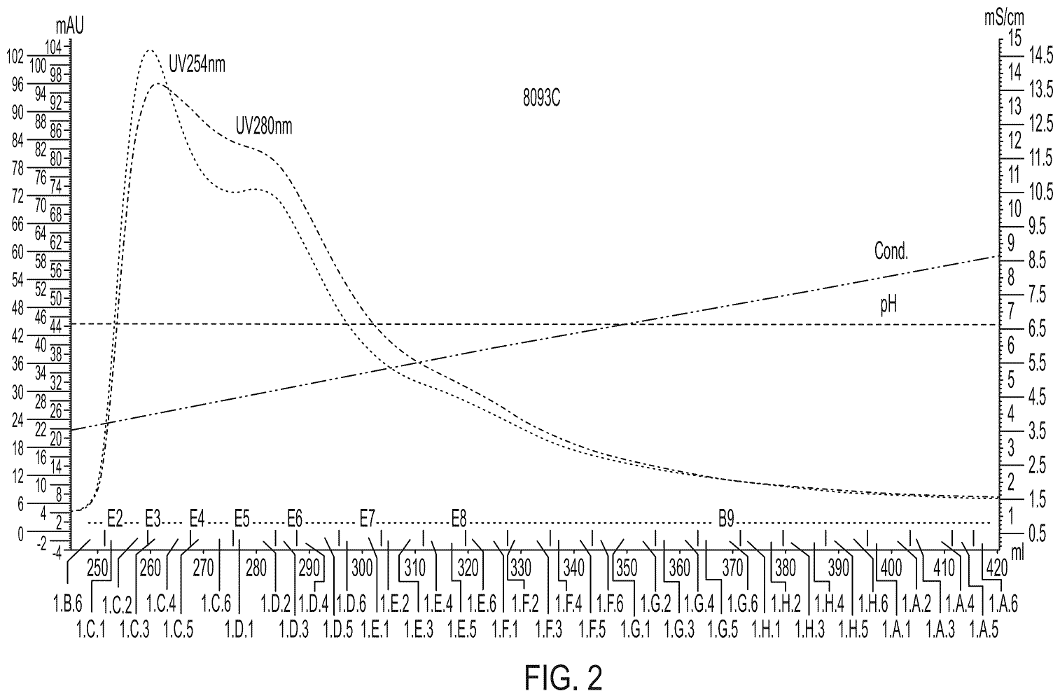

FIGS. 1 and 2 represent a chromatogram from the CEX run. It contains data of the complete run incl. regeneration procedure. Curves are shown for UV280 nm, UV254 nm, conductivity, pH, pressure, fluorescence and fractions. X-Axis: UV280 nm(left) conductivity (right), Y-Axis: Volume (ml).

TABLE 6

| | | | | | AAV8 ELISA | AAV8 ELISA | AAV8 | |
|---|---|---|---|---|---|---|---|---|
| | | FIX ddPCR | FIX ddPCR | | GTPA | GTPA | ELISA | |
| | Volume | [vg/mL] | [vg] | FIX ddPCR | [E+11 | total [E+11 | GTPA | Ratio |
| Sample code | [g] | ×10¹¹ | ×10¹¹ | Yield-[%] | cp/mL] | cp] | Yield [%] | vg/cp |
| 8093C_LOAD | 58.73 | 80.70 | 4739.51 | 100.00% | 314.10 | 18447.09 | 100.00% | 0.26 |
| 8093C_FT | 112.44 | <LOQ | — | — | <0.0319 | — | — | — |
| 8093C_W | n.a. | — | — | — | — | — | — | — |
| 8093C_E1 | 22.29 | 4.39 | 97.85 | 2.06% | 22.10 | 492.61 | 2.67% | 0.20 |
| 8093C_E2 | 7.45 | 52.20 | 388.89 | 8.21% | 31.80 | 236.91 | 1.28% | 1.64 |
| 8093C_E3 | 7.52 | 215.00 | 1616.80 | 34.11% | 124.00 | 932.48 | 5.05% | 1.73 |
| 8093C_E4 | 7.48 | 43.30 | 323.88 | 6.83% | 161.80 | 1210.26 | 6.56% | 0.27 |
| 8093C_E5 | 7.50 | 25.80 | 193.50 | 4.08% | 180.60 | 1354.50 | 7.34% | 0.14 |
| 8093C_E6 | 11.29 | 25.20 | 284.51 | 6.00% | 161.30 | 1821.08 | 9.87% | 0.16 |
| 8093C_E7 | 15.03 | 10.20 | 153.31 | 3.23% | 97.40 | 1463.92 | 7.94% | 0.10 |
| 8093C_E8 | 22.48 | 6.63 | 149.04 | 3.14% | 48.60 | 1092.53 | 5.92% | 0.14 |
| 8093C_E9 | 104.82 | 1.33 | 139.41 | 2.94% | 10.44 | 1094.32 | 5.93% | 0.13 |
| 8093C_NE | 98.35 | 2.34 | 230.14 | 4.86% | 11.60 | 1140.86 | 6.18% | 0.20 |
| 8093C_REG | 102.43 | 0.58 | 58.90 | 1.24% | 3.80 | 389.23 | 2.11% | 0.15 |
| Recovery | | | | 76.72% | | | 60.87% | |

The separation of AAV8 containing a vector genome of 2.6 kB on cation exchanger Capto S in presence of Calcium has shown high resolution of separation of empty and full AAV8 capsids. The yield of fraction E1, E2 and E3 was 44.4% in ddPCR and 9.1% in AAV8 Antigen.

### Example 3: AAV8, Vector Genome Size 2.6 kB, Capto S, in Absence of/or Chelated Divalent Cations

The following test procedure was undertaken. Note that all buffers disclosed in this example were made at room temperature and the pH of all buffers were measured at room temperature. First, a column containing Capto S Cation Exchanger Resin (Cat. 17-5441-01; Cytiva) ID 11 mm, with a bed height of 100 mm, an area of 0.95 cm², and a volume of approximately 9.5 ml, was equilibrated (activation) with 5 column volumes of a buffer comprising 1000 mM Sodium acetate, 2 mM EDTA, 0.005% Polysorbate 80 at a pH of 6.0. The column was then equilibrated with at least five column volumes of 30 mM Sodium acetate, 2 mM EDTA, 0.005% Polysorbate 80 at a pH of 6.0. The LOAD (AAV conditioned in 30 mM Sodium acetate, 2 mM EDTA, 0.005% Polysorbate 80 at a pH of 6.0) was applied onto the column containing Capto S Cation Exchanger Resin.

The column was then washed with five column volumes of 30 mM Sodium acetate, 2 mM EDTA, 0.005% Polysorbate 80 at a pH of 6.0.

For the elution a Gradient elution was then performed. The Gradient was performed with 40 column volumes of 30 mM Sodium acetate, 2 mM EDTA, 0.005% Polysorbate 80 at a pH of 6.0 to 200 mM Sodium acetate, 2 mM EDTA, 0.005% Polysorbate 80 at a pH of 6.0.

Post elution was performed with 10 column volumes of 1000 mM Sodium acetate, 2 mM EDTA, 0.005% Polysorbate 80 at a pH of 6.0.

For all steps the linear flow rate was 60 cm/h.

Buffer compositions are summarized in Table 7.

TABLE 7
Buffer composition for CEX-Separation

| Buffer code | Buffer content | pH |
|---|---|---|
| TWA (2M NaCl) | 2000 mmol NaCl | n.d |
| ADT1 | 30 mM NaAcetate | pH 6.0 ± 0.2 |
| | 2 mM EDTA | |
| | 0.005% Polysorbate 80 | |
| ADE | 200 mM NaAcetate | pH 6.0 ± 0.2 |
| | 2 mM EDTA | |
| | 0.005% Polysorbate 80 | |
| ADNE | 1000 mM NaAcetate | pH 6.0 ± 0.2 |
| | 2 mM EDTA | |
| | 0.005% Polysorbate 80 | |

Table 8 shows the chromatographic scheme for CEX-Separation

TABLE 8

| Step | | Buffer | Amount CV | Flow rate cm/h |
|---|---|---|---|---|
| 1 | Equilibration 1 | TWA (2M NaCl) | 5 | 60 |
| 2 | Equilibration 2 | AC1 | 5 | 60 |
| 3 | Product load | diluted affinity | x | 60 |
| | | Eluate | | |
| 4 | Wash 1 | AC1 | 5 | 60 |
| 5 | Elution | Gradient 0-100% | 40 | 60 |
| | | ACE | | |
| | | in AC1 | | |
| | Collecting | | 4 ml Fractions/No pooling | |
| 7 | Post elution | ACNE | 10 | 60 |

Table 9 shows the ratio of ddPCR/AAV8:AG

TABLE 9

| | Ratio ddPCR/AAV8:AG vg/cp |
|---|---|
| LOAD | 0.32 |
| E1 | 0.33 |
| E2 | 0.93 |
| E3 | 0.53 |
| E4 | 0.34 |
| E5 | 0.50 |
| E6 | 0.17 |
| E7 | 0.11 |
| E8 | 0.20 |

ddPCR: FIX-specific ddPCR/AAV8:AG determined with AAV8 Antigen ELISA

Table 10 shows the percentage of full and empty capsids determined with AUC.

TABLE 10

| | Area % | Area % | Area % | Area % | S | S | S | S |
|---|---|---|---|---|---|---|---|---|
| Name | empty | subpopulation | Full | Aggregates | empty | subpopulation | Full | Aggregates |
| LOAD | 36.4 | 0.8 | 56.6 | 6.2 | 64.9 | 74.3 | 88.8 | 98.2 |
| E2 | 31.5 | 1.4 | 61.5 | 5.6 | 64.1 | 75.2 | 88.0 | 101.7 |
| E3 | 31.9 | 1.7 | 63.8 | 2.5 | 63.2 | 76.9 | 86.3 | 97.4 |
| E5 | 46.3 | 0.5 | 46.1 | 7.2 | 64.1 | 72.6 | 86.3 | 98.2 |

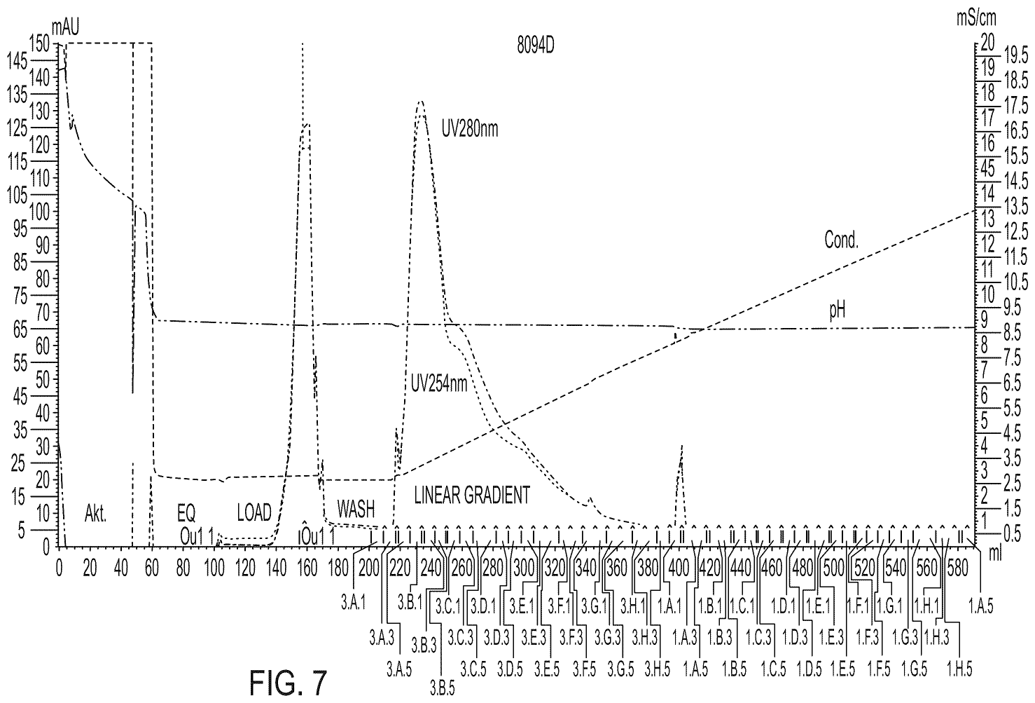

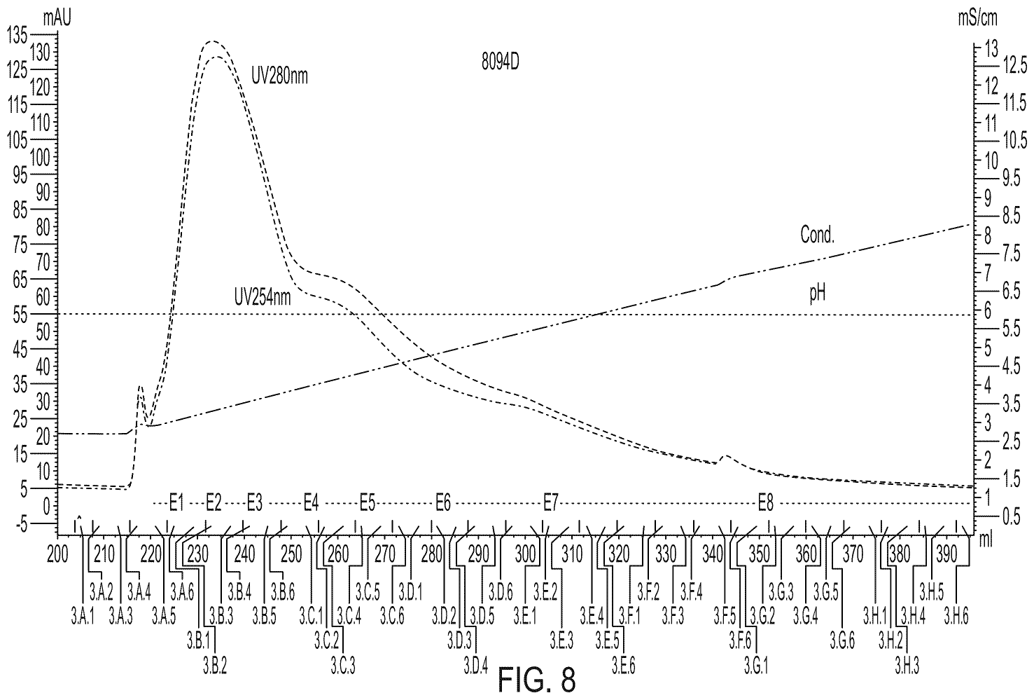

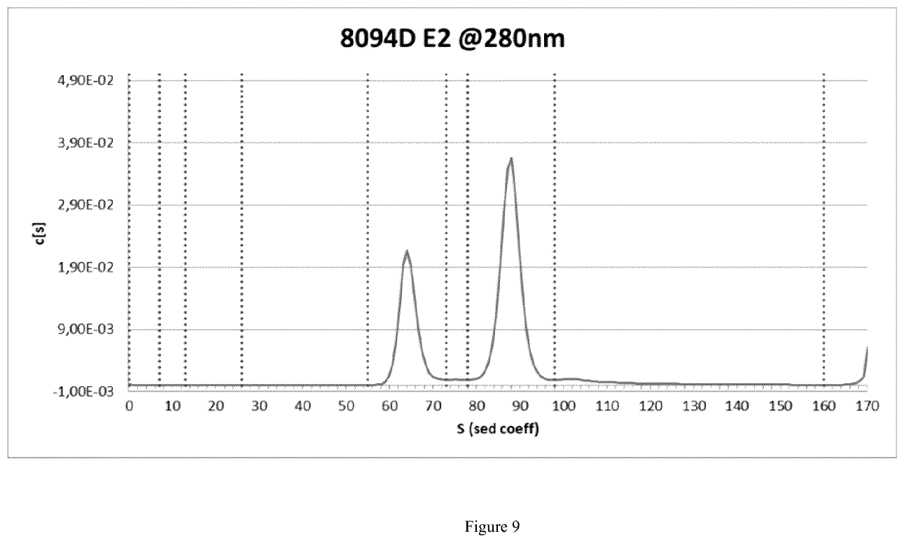

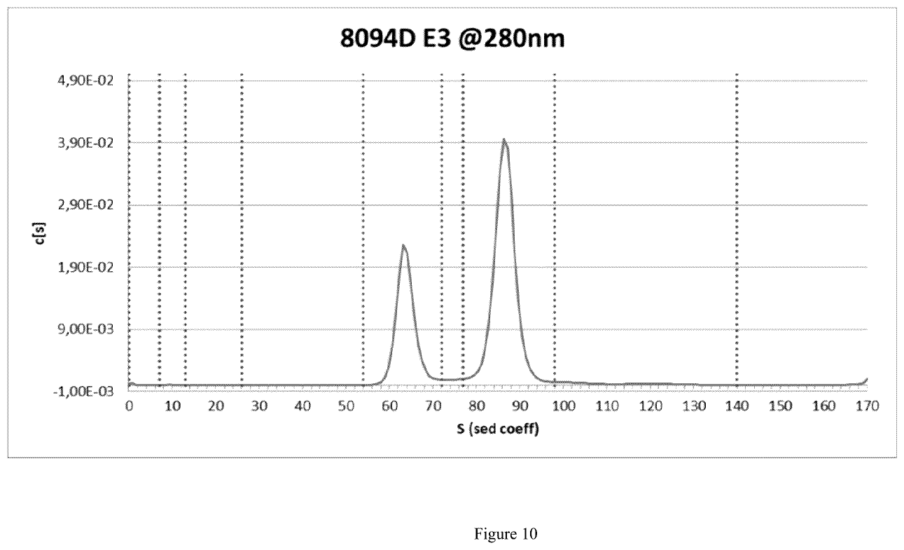

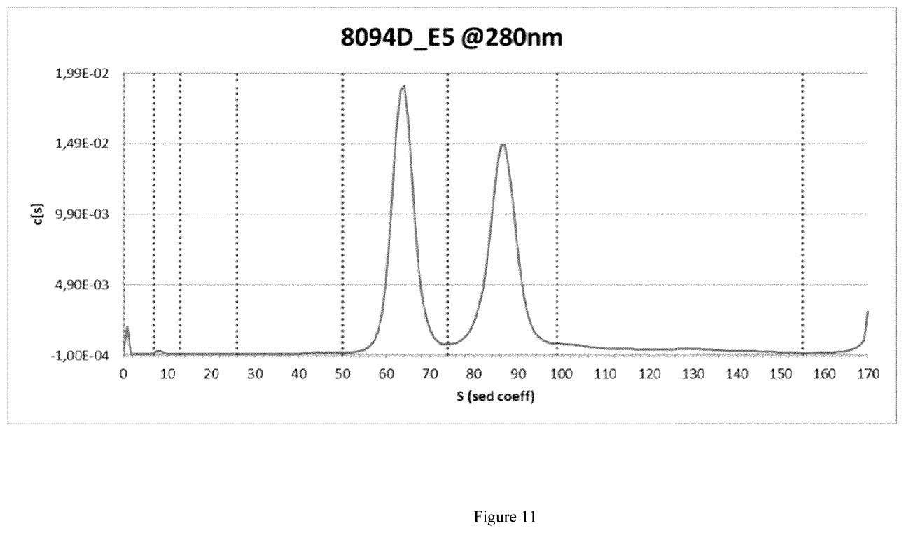

Results are shown in FIGS. 7-11 and yields are shown in Table 11.

FIGS. 7-11 represents a run with data of most interesting fractions:L_nativ=Affinity eluate, LOAD (Starting material=Affinity eluate dialysed against equilibration buffer), FT=Flow through, W=Wash, E=Eluate,E1-E3=Fractions of the peak (Chromatogramm). The ratio vg/cp which indicates the AAV's with the highest amount of full capsids (Full capsid fractions). The higher the value the higher is the amount of full capsids.

FIGS. 7 and 8 represent a chromatogram from the CEX run. It contains data of the complete run incl. regeneration procedure. Curves are shown for UV280 nm, UV254 nm, conductivity, pH, pressure, fluorescence and fractions. X-Axis: UV280 nm(left) conductivity (right), Y-Axis: Volume (ml).

TABLE 11

| | | | | | AAV8 ELISA | AAV8 ELISA | AAV8 | |
|---|---|---|---|---|---|---|---|---|
| | | FIX ddPCR | FIX ddPCR | | GTPA | GTPA | ELISA | |
| | Volume | [vg/mL] | [vg] | FIX ddPCR | [E+11 | total [E+11 | GTPA | Ratio |
| Sample code | [g] | ×10¹¹ | ×10¹¹ | Yield-[%] | cp/mL] | cp] | Yield [%] | vg/cp |
| 8094D_L_nativ | 60.00 | 86.50 | 5190.00 | — | 308.10 | 18486.00 | — | 0.28 |
| 8094D_ | 60.15 | 88.40 | 5317.26 | 100.00% | 275.20 | 16553.28 | 100.00% | 0.32 |
| LOAD | | | | | | | | |
| 8094D_FT | 109.07 | 6.74 | 735.13 | 13.83% | 30.32 | 3307.00 | 19.98% | 0.22 |
| 8094D_W | n.a. | — | — | — | n.a. | — | — | — |
| 8094D_E1 | 7.38 | 41.50 | 306.27 | 5.76% | 124.60 | 919.55 | 5.56% | 0.33 |
| 8094D_E2 | 7.55 | 229.00 | 1728.95 | 32.52% | 245.60 | 1854.28 | 11.20% | 0.93 |
| 8094D_E3 | 11.25 | 97.80 | 1100.25 | 20.69% | 185.00 | 2081.25 | 12.57% | 0.53 |
| 8094D_E4 | 11.21 | 45.20 | 506.69 | 9.53% | 133.50 | 1496.54 | 9.04% | 0.34 |
| 8094D_E5 | 11.31 | 63.80 | 721.58 | 13.57% | 127.40 | 1440.89 | 8.70% | 0.50 |
| 8094D_E6 | 18.79 | 15.00 | 281.85 | 5.30% | 90.80 | 1706.13 | 10.31% | 0.17 |
| 8094D_E7 | 29.93 | 6.08 | 181.97 | 3.42% | 53.80 | 1610.23 | 9.73% | 0.11 |
| 8094D_E8 | 66.92 | 1.96 | 131.16 | 2.47% | 9.70 | 649.12 | 3.92% | 0.20 |
| 8094D_NE | 98.35 | 0.17 | 17.01 | 0.32% | 1.38 | 136.12 | 0.82% | 0.13 |
| 8094D_ | 102.43 | 0.03 | 3.21 | 0.06% | 0.22 | 22.12 | 0.13% | 0.14 |
| REG | | | | | | | | |
| Recovery | | | | 107.46% | | | 91.97% | |

Adding EDTA instead of Calcium has shown significant lower resolution and shows reduced ability of the AAV8 construct to bind onto Capto S. Significant amounts of AAV8 was found in the Flow-Through.

### Example 4: AAV8, Vector Genome Size>4.8 kB, Capto S, in Presence of Calcium Preparation of the Load Material

AAV8 production was developed in a HEK293 cell line after transfection with a triple plasmid system containing encoding cDNA of the protein of interest and AAV8-. VP1.-VP2 and -VP3. The clarified cell free culture supernatant was concentrated and diafiltrated with Pall Omega T-Series Cassette 100 kDa. The viral particles were loaded onto a membrane adsorber (MustangQ. Pall Part Number XT140MSTGQP05) at nonbinding conditions. The obtained AAV8 containing flow through was used as Load for the following affinity purification step.

A column containing POROS™ CaptureSelect™ AAV8 Affinity Matrix (Cat. No. A30794. Thermo Fisher) ID 32 mm, with a bed height of 60 mm and a volume approx. 200 ml, was equilibrated with at least five column volumes of 50 mM TrisHCl and 125 mM NaCl at pH 8.5. The LOAD was applied onto the column containing POROS™ CaptureSelect™ AAV8 Affinity Matrix (Cat. No. A30794. Thermo Fisher). The column was then re-equilibrated with 5 column volumes of 50 mM TrisHCl and 125 mM NaCl at pH 8.5. The column was then washed with 5 column volumes of Wash 1 (W1): 100 mM Sodium Acetate and 0.1% Tween80 at pH 6.0. The column was then washed with 5 column volumes of Wash 2 (W2): 50 mM TrisHCl and 125 mM NaCl at pH 8.5. The column was then washed with 5 column volumes of Wash 3 (W3): 50 mM TrisHCl and 50% ethylene glycol at pH 8.5.

Elution was undertaken by applying 5 column volumes of the following elution buffer to the column: 50 mM TrisHCl, 50% ethylene glycol and 750 mM NaCl, at pH 8.0 and is described in more detail in Table A.

TABLE A
Flowrate

| Step | High pH LOAD | CV |
|---|---|---|
| 1. | 50 mM TrisHCl | >5 |
| | 125 mM NaCl | |
| | pH 8.5 | |
| 2. | Sample-Load | |
| | pH 8.5 | |
| 3. | 50 mM TrisHCl | 5 |
| | 125 mM NaCl | |
| | pH 8.5 | |
| 4. | 100 mM NaAcetat | 5 |
| | 0.1% Tween80 | |
| | pH 6.0 | |
| 5. | 50 mM TrisHCl | 5 |
| | 125 mM NaCl | |
| | pH 8.5 | |
| 6. | 50 mM TrisHCl | 5 |
| | 50% Ethylenglycol | |
| | pH 8.5 | |
| 7. | ELUTION | 5 |
| | 50 mM TrisHCl | |
| | 50% Ethylenglycol | |
| | 750 mM NaCl | |
| | pH 8.0 | |
| 8. | Post-Elution | 5 |
| | 50 mM TrisHCl | |
| | 50% Ethylenglycol | |
| | 2000 mM NaCl | |
| | pH 8.0 | |

A buffer exchange of the eluate into 30 mM NaAcetate, 2 mM CaAcetate, 0.005% Polysorbate 80, pH 6.0 was performed with VIVACELL100 (10K) cartridges (Sartorius) to provide proper binding properties for all AAV-Subtypes on cation exchanger (CEX).

The following test procedure was undertaken. Note that all buffers disclosed in this example were made at room temperature and the pH of all buffers were measured at room temperature. First, a column containing Capto S Cation Exchanger Resin (Cat. 17-5441-01; Cytiva) ID 11 mm, with a bed height of 101 mm, an area of 0.95 cm², and a volume of approximately 9.6 ml, was equilibrated (activation) with 5 column volumes of a buffer comprising 1000 mM Sodium acetate, 2 mM Calcium acetate, 0.005% Polysorbate 80 at a pH of 6.0. The column was then equilibrated with at least five column volumes of 30 mM Sodium acetate, 2 mM Calcium acetate, 0.005% Polysorbate 80 at a pH of 6.0. The LOAD (AAV conditioned in 30 mM Sodium acetate, 2 mM Calcium acetate, 0.005% Polysorbate 80 at a pH of 6.0) was applied onto the column containing Capto S Cation Exchanger Resin.

The column was then washed with five column volumes of 30 mM Sodium acetate, 2 mM Calcium acetate, 0.005% Polysorbate 80 at a pH of 6.0.

For the elution a Gradient elution was then performed. The Gradient was performed with 40 column volumes of 30 mM Sodium acetate, 2 mM Calcium acetate, 0.005% Polysorbate 80 at a pH of 6.0 to 200 mM Sodium acetate, 2 mM Calcium acetate, 0.005% Polysorbate 80 at a pH of 6.0. Post elution was performed with 10 column volumes of 1000 mM Sodium acetate, 2 mM Calcium acetate, 0.005% Polysorbate 80 at a pH of 6.0.

For all steps the linear flow rate was 60 cm/h.

Buffer compositions are summarized in Table 12.

TABLE 12
Buffer composition for CEX-Separation

| Buffer | Buffer content | pH |
|---|---|---|
| TWA (2M NaCl) | 2000 mmol NaCl | n.d |
| AC1 | 30 mM NaAcetate | pH 6.0 ± 0.2 |
| | 2 mM CaAcetate | |
| | 0.005% Polysorbate 80 | |
| ACE | 200 mM NaAcetate | pH 6.0 ± 0.2 |
| | 2 mM CaAcetate | |
| | 0.005% Polysorbate 80 | |
| ACNE | 1000 mM NaAcetate | pH 6.0 ± 0.2 |
| | 2 mM CaAcetate | |
| | 0.005% Polysorbate 80 | |

Table 13 shows the chromatographic scheme for CEX-Separation

TABLE 13

| Step | | Buffer | Amount CV | Flow rate cm/h |
|---|---|---|---|---|
| 1 | Equilibration 1 | TWA (2M NaC1) | 5 | 60 |
| 2 | Equilibration 2 | AC1 | 5 | 60 |
| 3 | Product load | diluted affinity | x | 60 |
| | | Eluate | | |
| 4 | Wash 1 | AC1 | 5 | 60 |
| 5 | Elution | Gradient 0-100% | 40 | 60 |
| | | ACE in AC1 | | |
| | Collecting | | 4 ml Fractions | |
| 7 | Post elution | ACNE | 10 | 60 |

Table 14 shows the ratio of ddPCR/AAV8:AG

TABLE 14

| | Ratio ddPCR/AAV8:AG vg/cp |
|---|---|
| LOAD | 0.066 |
| E1 | 0.277 |
| E2 | 0.070 |
| E3 | 0.043 |
| E4 | 0.042 |
| E5 | 0.061 |
| E6 | 0.070 |
| E7 | 0.107 |
| E8 | 0.178 |
| E9 | 0.169 |

ddPCR: human coagulation VIII -specific ddPCR/AAV8:AG determined with AAV8 Antigen ELISA

Table 15 shows the percentage of full and empty capsids determined with AUC.

TABLE 15

| | Area % | Area % | Area % | Area % | S | S | S | S |
|---|---|---|---|---|---|---|---|---|
| Name | empty | subpopulation | Full | Aggregates | empty | subpopulation | Full | Aggregates |
| LOAD | 68.2 | 0.4 | 18.7 | 12.7 | 64.1 | 72.6 | 84.6 | 92.3 |
| E2 | 53.6 | 7.4 | 16.9 | 22.1 | 64.1 | 80.3 | 88.0 | 100.8 |
| E3 | 66.1 | 11.6 | 12.7 | 9.6 | 64.1 | 76.9 | 95.7 | 99.9 |
| E4 | 68.3 | 12.0 | 9.1 | 10.6 | 63.2 | 79.4 | 89.7 | 97.4 |
| E5 | 62.8 | 4.4 | 16.8 | 16.0 | 63.2 | 75.2 | 91.4 | 99.1 |

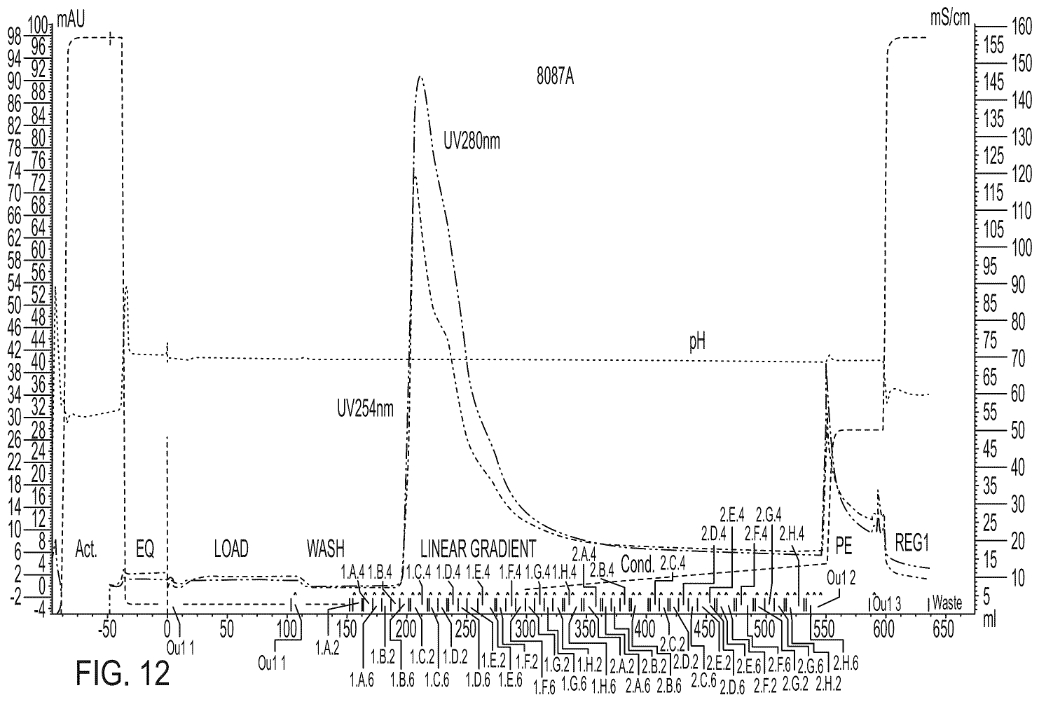

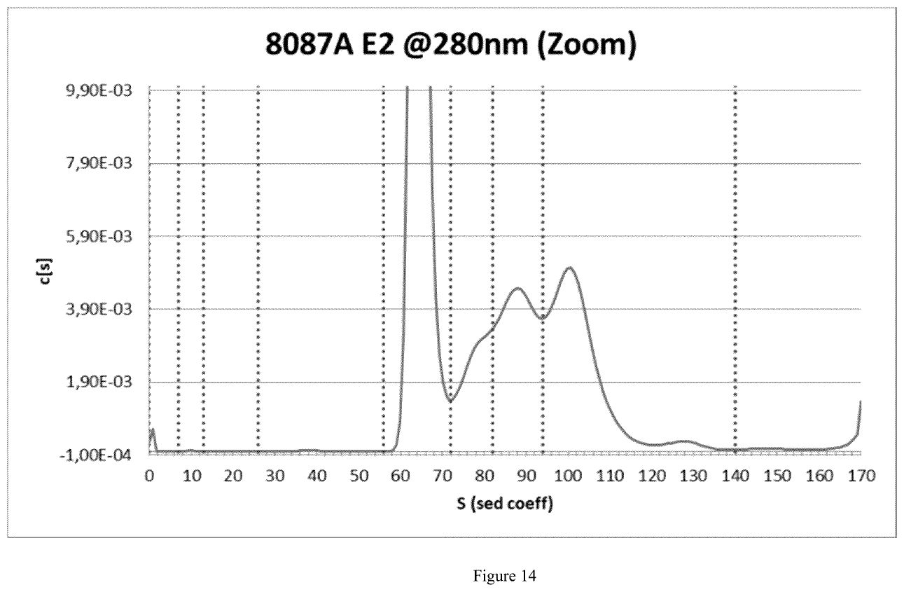

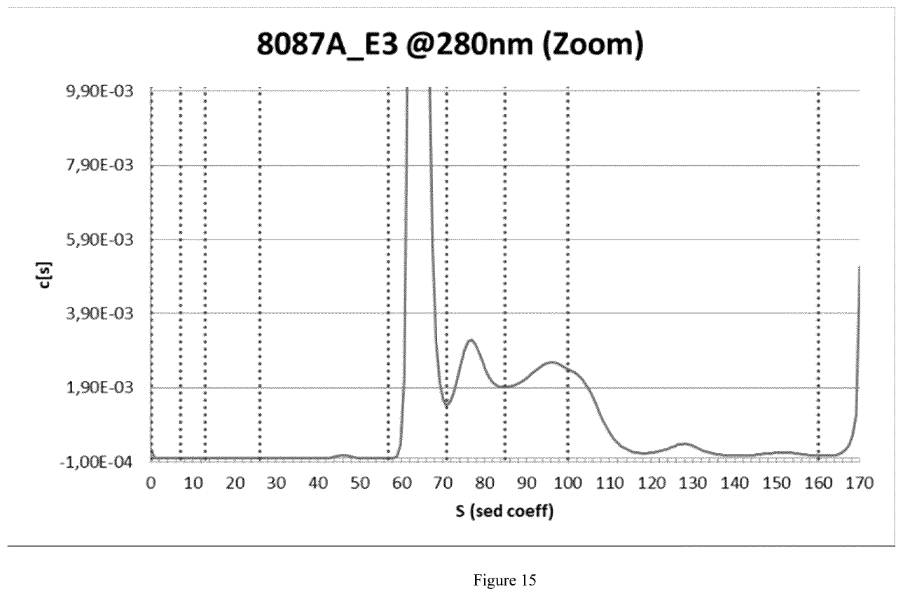

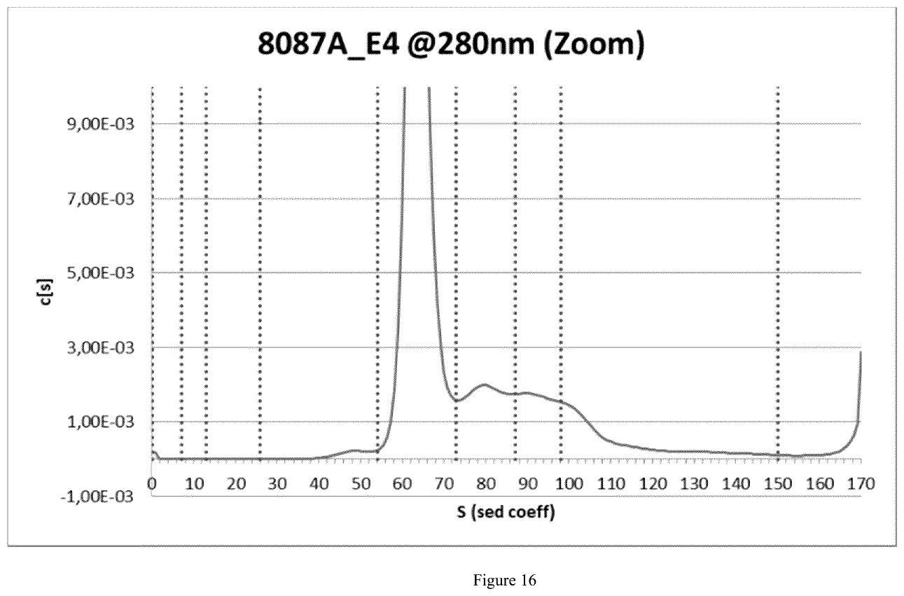

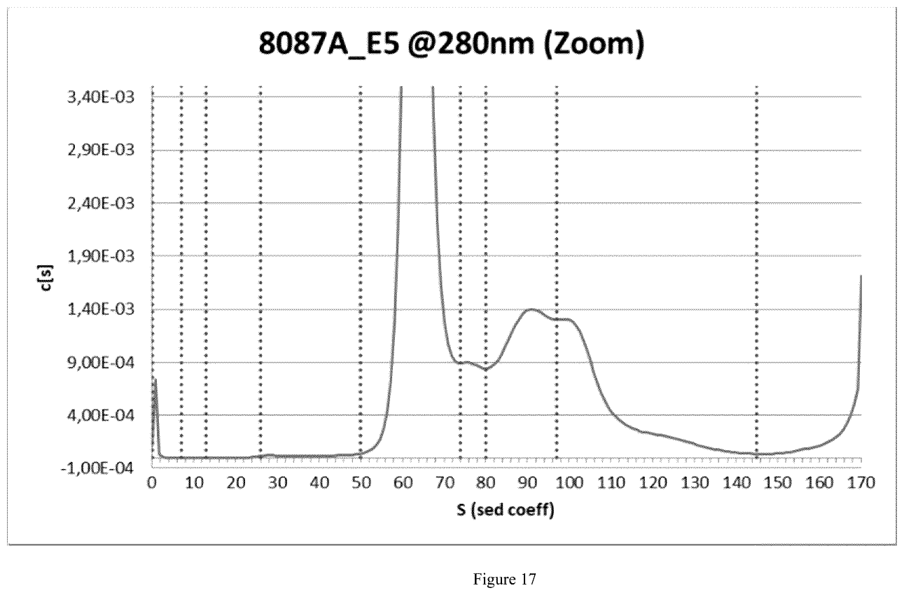

Results are shown in FIGS. 12-17 and yields are shown in Table 16.

FIGS. 12-17 represents a run with data of most of the fractions:L nativ=Affinity eluate. LOAD=(Starting material), FT=Flow through, W=Wash, E=Eluate, E1-E5=Fractions of the peak (Chromatogramm). The ratio vg/cp which indicates the AAV's with the highest amount of full capsids (Full capsid fractions). The higher the value the higher is the amount of full capsids.

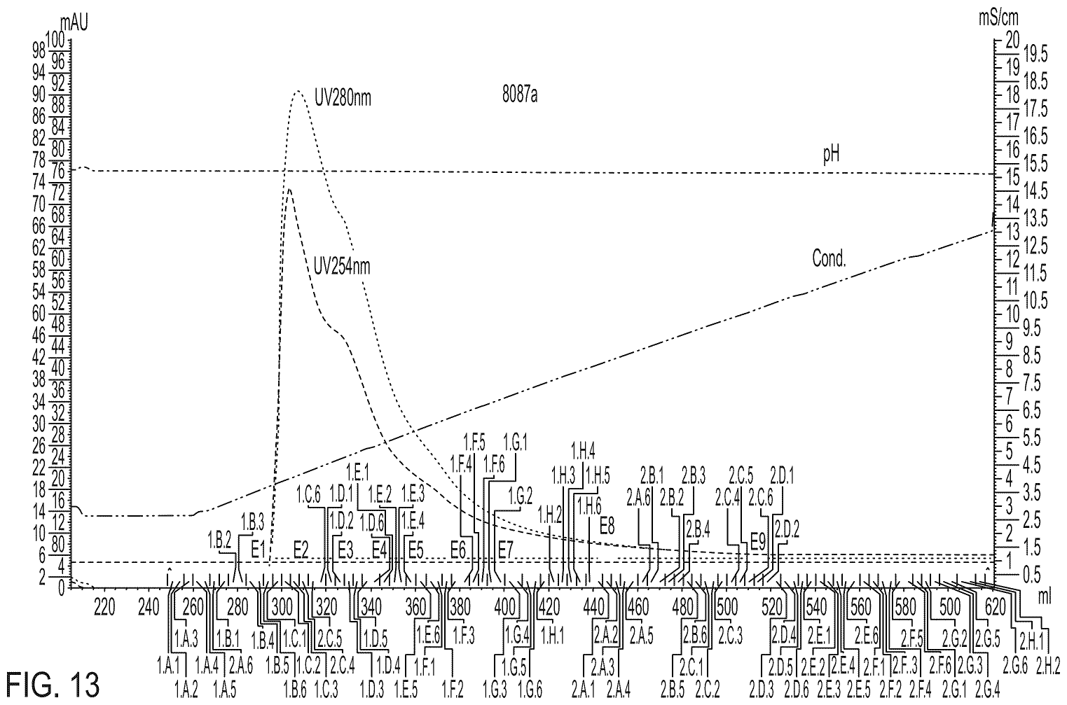

FIGS. 12 and 13 represent a chromatogram from the CEX run. It contains data of the complete run incl. regeneration procedure. Curves are shown for UV280 nm, UV254 nm, conductivity, pH, pressure, fluorescence and fractions. X-Axis: UV280 nm(left) conductivity (right), Y-Axis: Volume (ml).

TABLE 16

| | | duplex | | | | | | |
|---|---|---|---|---|---|---|---|---|
| | | ddPCR | duplex | duplex | | | | |
| | | FVIII/ITR | ddPCR | ddPCR | | | | |
| | | ITR | FVIII/ITR | FVIII/ITR | | AAV8 ELISA | AAV8 | |
| | Volumen | [vg/mL] | ITR [vg] | ITR Yield- | AAV8 ELISA | total | ELISA | Ratio |
| Sample code | [g] | ×10¹¹ | ×10¹¹ | [%] | [cp/mL] ×10¹¹ | [cp] ×10¹¹ | Yield [%] | vg/cp |
| 8087A_L nativ | 66.04 | 23.3 | 1538.73 | — | 277.2 | 18306.29 | — | 0.07 |
| 8087A_LOAD | 98.99 | 13.3 | 1316.57 | 100.00% | 158.65 | 15704.76 | 100.00% | 0.07 |
| 8087A_FT | 150.09 | LOQ | — | — | <0.0319 | — | — | — |
| 8087A_VE | 11.42 | — | — | — | 0.30 | 3.43 | 0.02% | — |
| 8087A_E1 | 3.81 | 2.36 | 8.99 | 0.68% | 7.10 | 27.05 | 0.17% | 0.28 |
| 8087A_E2 | 23.06 | 15.90 | 366.65 | 27.85% | 149.6 | 3449.78 | 21.97% | 0.07 |
| 8087A_E3 | 15.04 | 8.20 | 123.33 | 9.37% | 132.6 | 1994.30 | 12.70% | 0.04 |
| 8087A_E4 | 15.27 | 4.87 | 74.36 | 5.65% | 83.0 | 1267.41 | 8.07% | 0.04 |
| 8087A_E5 | 15.03 | 3.52 | 52.91 | 4.02% | 47.0 | 706.41 | 4.50% | 0.06 |
| 8087A_E6 | 19.22 | 2.52 | 48.43 | 3.68% | 30.6 | 588.13 | 3.74% | 0.07 |
| 8087A_E7 | 26.85 | 1.83 | 49.14 | 3.73% | 16.5 | 443.29 | 2.82% | 0.11 |
| 8087A_E8 | 57.54 | 1.17 | 67.32 | 5.11% | 6.8 | 391.27 | 2.49% | 0.18 |
| 8087A_E9 | 139.39 | 0.49 | 68.72 | 5.22% | 2.8 | 396.56 | 2.53% | 0.17 |
| 8087A_NE | 49.42 | 1.46 | 72.15 | 5.48% | 17.8 | 879.68 | 5.60% | 0.05 |
| 8087A_Strip | 49.55 | 0.432 | 21.41 | 1.63% | 8.5 | 423.16 | 2.69% | 0.03 |
| Recovery | | | | 72.42% | | | 67.31% | |

### Example 5: AAV8 Vector Genome Size 2.6 kB, Eshmuno S, in Presence of Calcium

The following test procedure was undertaken. Note that all buffers disclosed in this example were made at room temperature and the pH of all buffers were measured at room temperature. First, a column containing Eshmuno S Cation Exchanger Resin (Cat. 1.20078; Merck-Millipore) ID 11 mm, with a bed height of 100 mm, an area of 0.95 cm², and a volume of approximately 9.5 ml, was equilibrated (activation) with 5 column volumes of a buffer comprising 1000 mM Sodium acetate, 2 mM Calcium acetate, 0.005% Polysorbate 80 at a pH of 6.0. The column was then equilibrated with at least five column volumes of 30 mM Sodium acetate, 2 mM Calcium acetate, 0.005% Polysorbate 80 at a pH of 6.0. The LOAD (AAV conditioned in 30 mM Sodium acetate, 2 mM Calcium acetate, 0.005% Polysorbate 80 at a pH of 6.0) was applied onto the column containing Eshmuno S Cation Exchanger Resin.

The column was then washed with five column volumes of 30 mM Sodium acetate, 2 mM Calcium acetate, 0.005% Polysorbate 80 at a pH of 6.0.

For the elution a Gradient elution was then performed. The Gradient was performed with 40 column volumes of 30 mM Sodium acetate, 2 mM Calcium acetate, 0.005% Polysorbate 80 at a pH of 6.0 to 200 mM Sodium acetate, 2 mM Calcium acetate, 0.005% Polysorbate 80 at a pH of 6.0. Post elution was performed with 10 column volumes of 1000 mM Sodium acetate, 2 mM Calcium acetate, 0.005% Polysorbate 80 at a pH of 6.0.

For all steps the linear flow rate was 60 cm/h.

Buffer compositions are summarized in Table 17.

TABLE 17
Buffer composition for CEX-Separation

| Buffer code | Buffer content | pH |
|---|---|---|
| TWA (2M NaCl) | 2000 mmol NaCl | n.d |
| AC1 | 30 mM NaAcetate | pH 6.0 ± 0.2 |
| | 2 mM CaAcetate | |
| | 0.005% Polysorbate 80 | |
| ACE | 200 mM NaAcetate | pH 6.0 ± 0.2 |
| | 2 mM CaAcetate | |
| | 0.005% Polysorbate 80 | |
| ACNE | 1000 mM NaAcetate | pH 6.0 ± 0.2 |
| | 2 mM CaAcetate | |
| | 0.005% Polysorbate 80 | |

Table 18 shows the chromatographic scheme for CEX-Separation

TABLE 18

| Step | | Buffer | Amount CV | Flow rate cm/h |
|---|---|---|---|---|
| 1 | Equilibration 1 | TWA (2M NaC1) | 5 | 60 |
| 2 | Equilibration 2 | AC1 | 5 | 60 |
| 3 | Product load | diluted affinity | x | 60 |
| | | Eluate | | |
| 4 | Wash 1 | AC1 | 5 | 60 |
| 5 | Elution | Gradient 0-100% | 40 | 60 |
| | | ACE2 in AC1 | | |
| | Collecting | | 4 ml Fractions | |
| 7 | Post elution | ACNE | 10 | 60 |

Table 19 shows the ratio of ddPCR/AAV8:AG

TABLE 19

| Sample code | Ratio ddPCR/AAV8:AG vg/cp |
|---|---|
| LOAD | 0.28 |
| E1 | 0.65 |
| E2 | 0.57 |
| E3 | 0.31 |
| E4 | 0.23 |
| E5 | 0.22 |
| E6 | 0.16 |
| E7 | 0.20 |
| E8 | 0.20 |
| E9 | 0.28 |

dPCR: FIX-specific ddPCR/AAV8:AG determined with AAV8 Antigen ELISA

Table 20 shows the percentage of full and empty capsids determined with AUC.

TABLE 20

| | Area % | Area % | Area % | Area % | S | S | S | S |
|---|---|---|---|---|---|---|---|---|
| Name | empty | subpopulation | Full | Aggregates | empty | subpopulation | Full | Aggregates |
| LOAD | 36.4 | 0.8 | 56.6 | 6.2 | 64.9 | 74.3 | 88.8 | 98.2 |
| E1 | 4.8 | 0.2 | 92.5 | 2.4 | 67.5 | 74.3 | 86.3 | 112.8 |
| E2 | 9.7 | 2.8 | 77.4 | 10.1 | 64.1 | 69.2 | 88.0 | 101.7 |
| E3 | 28.1 | 5.9 | 58.7 | 7.3 | 64.1 | 74.3 | 88.0 | 101.7 |
| E4 | 34.5 | 1.1 | 60.0 | 4.5 | 64.1 | 69.2 | 87.1 | 103.4 |
| E5 | 35.0 | 3.2 | 55.3 | 6.6 | 63.2 | 75.2 | 87.1 | 101.7 |

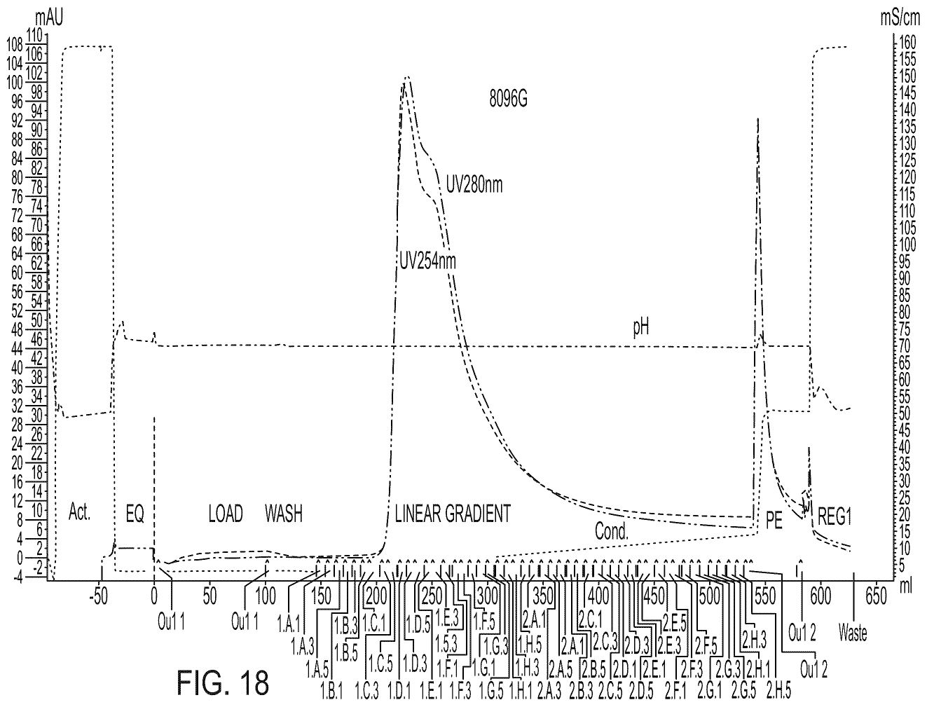

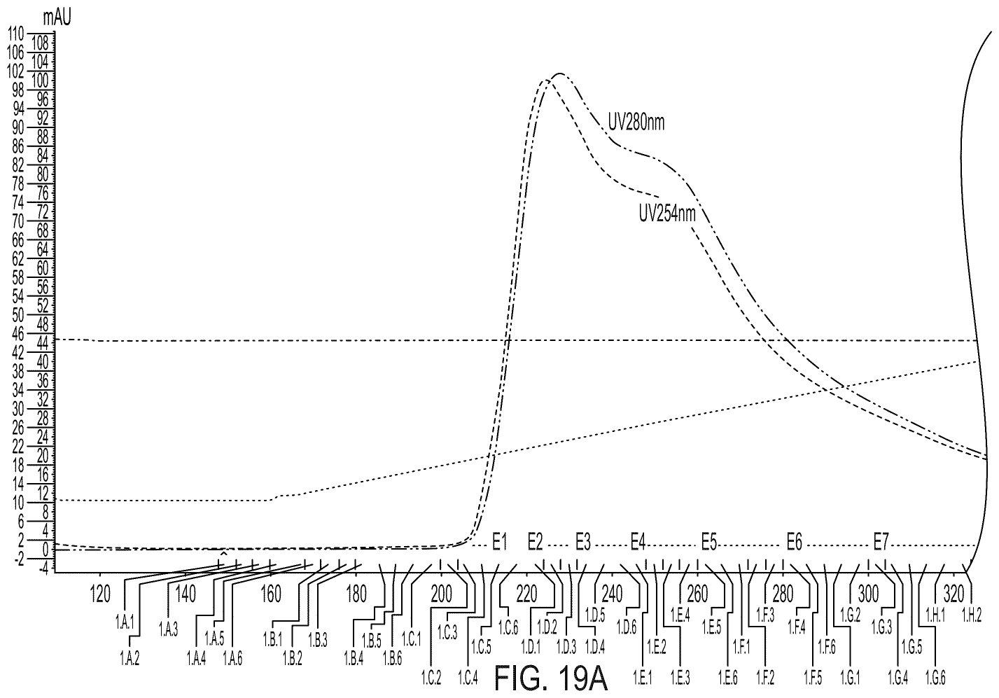

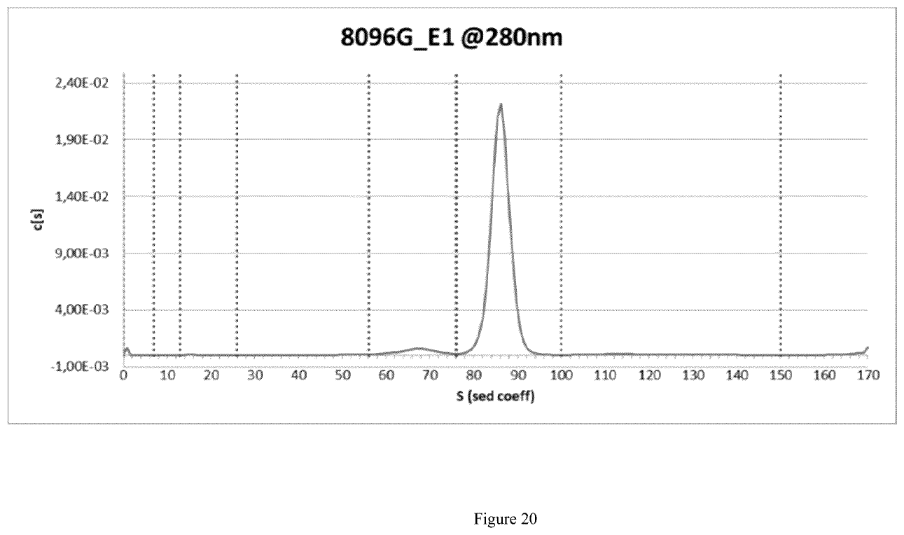

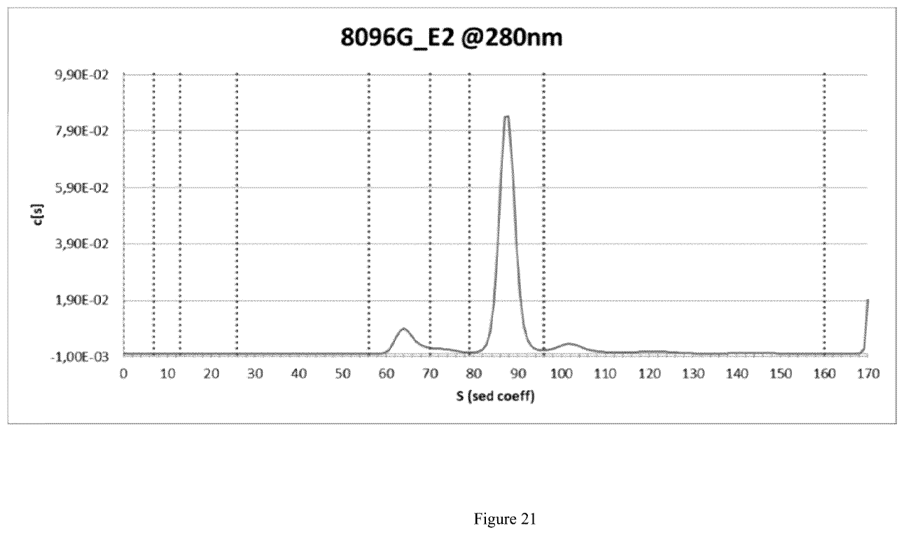

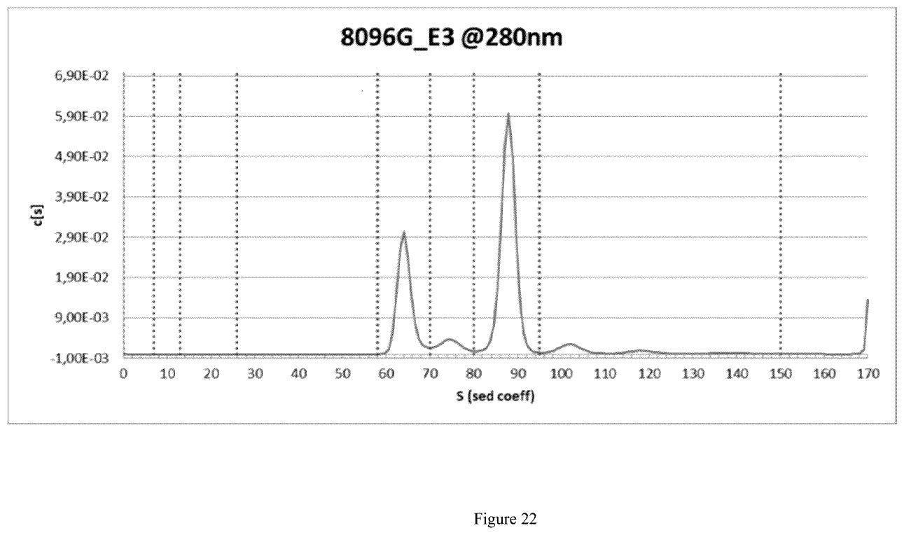

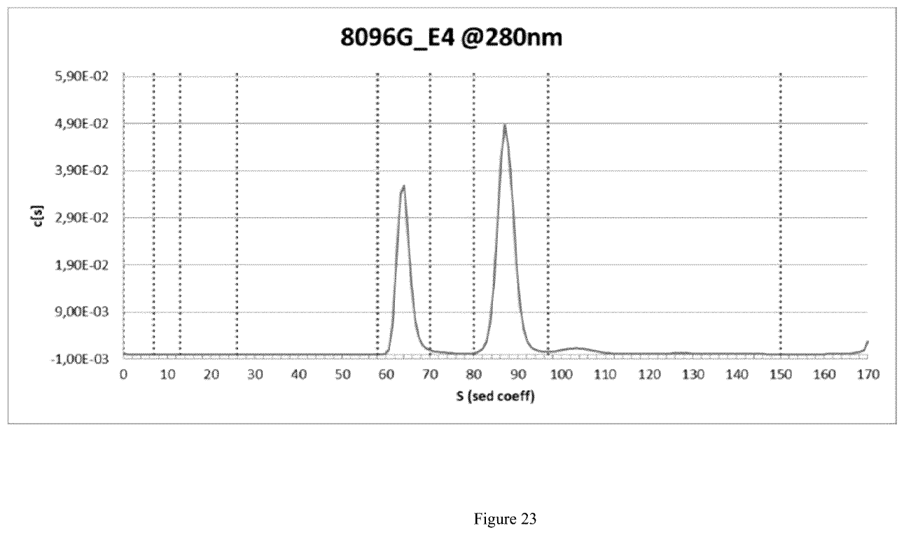

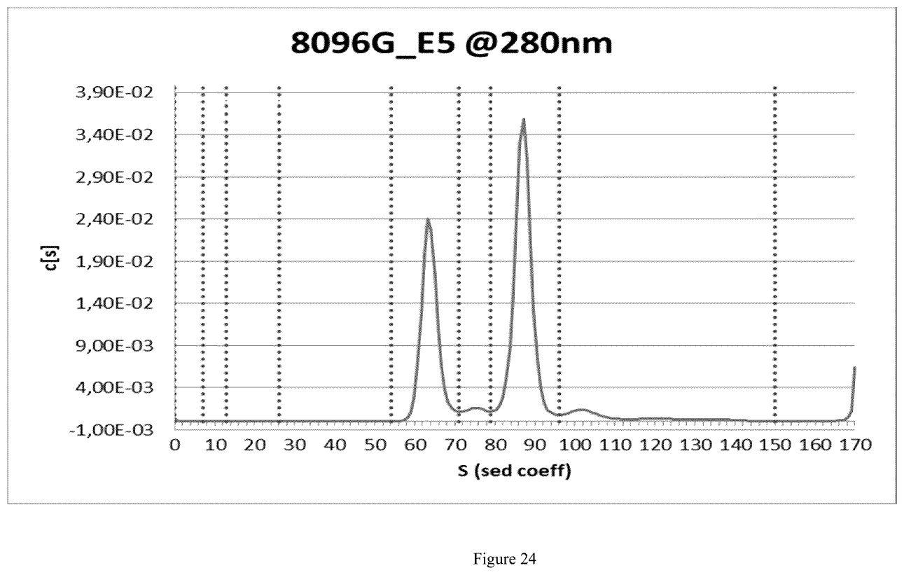

Results are shown in FIGS. 18-24 and yields are shown in Table 21.

FIGS. 18-24 represents a run with data of most of the fractions: L nativ=AAV8 affiniy eluate. Load (Starting material, after dialysis into equilibration buffer), FT=Flow through, W=Wash, E=Eluate, E1-E9=Fractions of the peak (Chromatogramm). The ratio vg/cp which indicates the AAV's with the highest amount of full capsids (Full capsid fractions). The higher the value the higher is the amount of full capsids.

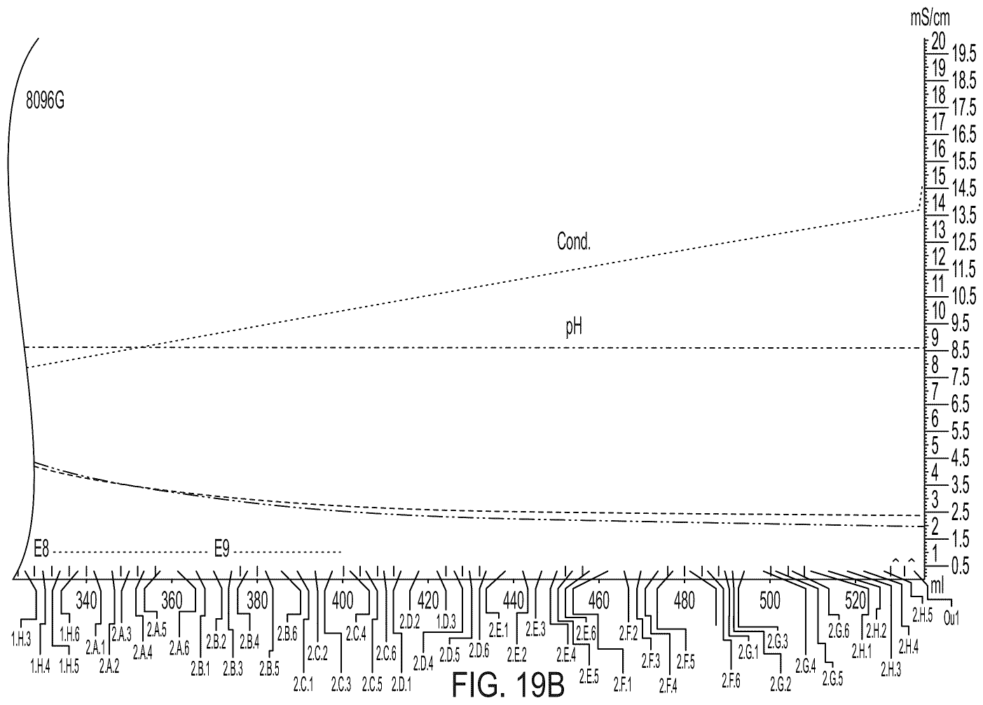

FIGS. 18 and 19 represent a chromatogram from the CEX run. It contains data of the complete run incl. regeneration procedure. Curves are shown for UV280 nm, UV254 nm, conductivity, pH, pressure, fluorescence and fractions. X-Axis: UV280 nm(left) conductivity (right), Y-Axis: Volume (ml).

TABLE 21

| | | ddPCR FIX | | | | AAV8 ELISA | | |
|---|---|---|---|---|---|---|---|---|
| | Volumen | [vg/mL] | ddPCR FIX | ddPCR FIX | AAV8 ELISA | total | AAV8 ELISA | Ratio |
| Sample code | [g] | ×10¹¹ | [vg] ×10¹¹ | Yield [%] | [cp/mL] ×10¹¹ | [cp] ×10¹¹ | Yield [%] | vg/cp |
| 8096G_L nativ | 66.08 | 87.0 | 5748.96 | — | 306.5 | 20253.52 | — | 0.28 |
| 8096G_LOAD | 95.24 | 44.7 | 4257.23 | 100.00% | 159.95 | 15233.64 | 100.00% | 0.28 |
| 8096G_FT | 146.08 | LOQ | — | — | <0.0319 | — | — | — |
| 8096G_VE | 10.07 | — | — | — | 1.19 | 12.00 | 0.08% | — |
| 8096G_E1 | 7.53 | 13.1 | 98.64 | 2.32% | 20.3 | 152.86 | 1.00% | 0.65 |
| 8096G_E2 | 7.61 | 51.4 | 391.15 | 9.19% | 90.8 | 690.99 | 4.54% | 0.57 |
| 8096G_E3 | 15.60 | 44.6 | 695.76 | 16.34% | 145.6 | 2271.36 | 14.91% | 0.31 |
| 8096G_E4 | 11.33 | 31.4 | 355.76 | 8.36% | 138.2 | 1565.81 | 10.28% | 0.23 |
| 8096G_E5 | 19.09 | 27.7 | 528.79 | 12.42% | 124.2 | 2370.98 | 15.56% | 0.22 |
| 8096G_E6 | 19.12 | 13.6 | 260.03 | 6.11% | 83.0 | 1586.96 | 10.42% | 0.16 |
| 8096G_E7 | 23.10 | 9.53 | 220.14 | 5.17% | 46.8 | 1081.08 | 7.10% | 0.20 |
| 8096G_E8 | 30.53 | 4.98 | 152.04 | 3.57% | 24.8 | 757.14 | 4.97% | 0.20 |
| 8096G_E9 | 165.25 | 2.33 | 385.03 | 9.04% | 8.3 | 1371.58 | 9.00% | 0.28 |
| 8096G_NE | 48.88 | 7.53 | 368.07 | 8.65% | 37.7 | 1842.78 | 12.10% | 0.20 |
| 8096G_Strip | 50.98 | 1.28 | 65.25 | 1.53% | 7.8 | 395.10 | 2.59% | 0.17 |
| Recovery | | | | 82.70% | | | 92.55% | |

### Example 6: AAV8, Vector Genome Size 2.6 kB, Capto S, in Presence of Calcium

The following test procedure was undertaken. Note that all buffers disclosed in this example were made at room temperature and the pH of all buffers were measured at room temperature. First, a column containing Capto S Cation Exchanger Resin (Cat. 17-5441-01; Cytiva) ID 11 mm, with a bed height of 100 mm, an area of 0.95 cm², and a volume of approximately 9.5 ml, was equilibrated (activation) with 5 column volumes of a buffer comprising 1000 mM Sodium acetate, 2 mM Calcium acetate, 0.005% Polysorbate 80 at a pH of 6.0. The column was then equilibrated with at least five column volumes of 30 mM Sodium acetate, 2 mM Calcium acetate, 0.005% Polysorbate 80 at a pH of 6.0. The LOAD (AAV conditioned in 30 mM Sodium acetate, 2 mM Calcium acetate, 0.005% Polysorbate 80 at a pH of 6.0) was applied onto the column containing Capto S Cation Exchanger Resin.

The column was then washed with five column volumes of 30 mM Sodium acetate, 2 mM Calcium acetate, 0.005% Polysorbate 80 at a pH of 6.0.

For the elution a Gradient elution was then performed. The Gradient was performed with 40 column volumes of 30 mM Sodium acetate, 2 mM Calcium acetate, 0.005% Polysorbate 80 at a pH of 6.0 to 80 mM Sodium acetate, 2 mM Calcium acetate, 0.005% Polysorbate 80 at a pH of 6.0. Post elution was performed with 10 column volumes of 1000 mM Sodium acetate, 2 mM Calcium acetate, 0.005% Polysorbate 80 at a pH of 6.0.

For all steps the linear flow rate was 60 cm/h.

Buffer compositions are summarized in Table 22.

TABLE 22
Buffer composition for CEX-Separation

| Buffer code | Buffer content | pH |
|---|---|---|
| TWA (2M NaCl) | 2000 mmol NaC1 | n.d |
| AC1 | 30 mM NaAcetate | pH 6.0 ± 0.2 |
| | 2 mM CaAcetate | |
| | 0.005% Polysorbate 80 | |
| ACE2 | 80 mM NaAcetate | pH 6.0 ± 0.2 |
| | 2 mM CaAcetate | |
| | 0.005% Polysorbate 80 | |
| ACNE | 1000 mM NaAcetate | pH 6.0 ± 0.2 |
| | 2 mM CaAcetate | |
| | 0.005% Polysorbate 80 | |

Table 23 shows the chromatographic scheme for CEX-Separation

TABLE 23

| Step | | Buffer | Amount CV | Flow rate cm/h |
|---|---|---|---|---|
| 1 | Equilibration 1 | TWA (2M NaC1) | 5 | 60 |
| 2 | Equilibration 2 | AC1 | 5 | 60 |
| 3 | Product load | diluted affinity | x | 60 |
| | | Eluate | | |
| 4 | Wash 1 | AC1 | 5 | 60 |
| 5 | Elution | Gradient 0-100% | 40 | 60 |
| | | ACE2 in AC1 | | |
| | Collecting | | 4 ml Fractions | |
| 7 | Post elution | ACNE | 10 | 60 |

Table 24 shows the ratio of ddPCR/AAV8:AG

TABLE 24

| Sample code | Ratio ddPCR/AAV8:AG vg/cp |
|---|---|
| LOAD | 0.28 |
| E1 | 0.78 |
| E2 | 0.64 |
| E3 | 0.54 |
| E4 | 0.39 |
| E5 | 0.22 |
| E6 | 0.22 |

ddPCR: FIX-specific ddPCR/AAV8:AG determined with AAV8 Antigen ELISA

Table 25 shows the percentage of full and empty capsids determined with AUC.

TABLE 25

| | Area % | Area % | Area % | Area % | S | S | S | S |
|---|---|---|---|---|---|---|---|---|
| Name | empty | subpopulation | Full | Aggregates | empty | subpopulation | Full | Aggregates |
| LOAD | 39.7 | 1.0 | 50.6 | 8.8 | 64.9 | 74.3 | 88.8 | 98.2 |
| E1 | 5.3 | 0.5 | 89.1 | 5.0 | 68.3 | 73.5 | 86.3 | 105.1 |
| E2 | 7.4 | 0.5 | 81.6 | 10.4 | 64.1 | 70.9 | 86.3 | 99.9 |
| E3 | 17.5 | 0.1 | 75.7 | 6.7 | 64.9 | 75.2 | 88.0 | 106.8 |
| E4 | 33.5 | 0.1 | 63.0 | 3.3 | 64.9 | 73.5 | 88.0 | 110.2 |
| E5 | 45.7 | 0.7 | 48.9 | 4.7 | 64.9 | 76.9 | 88.0 | 103.4 |
| E6 | 42.5 | 0.6 | 54.3 | 2.7 | 64.1 | 76.9 | 87.1 | 108.5 |

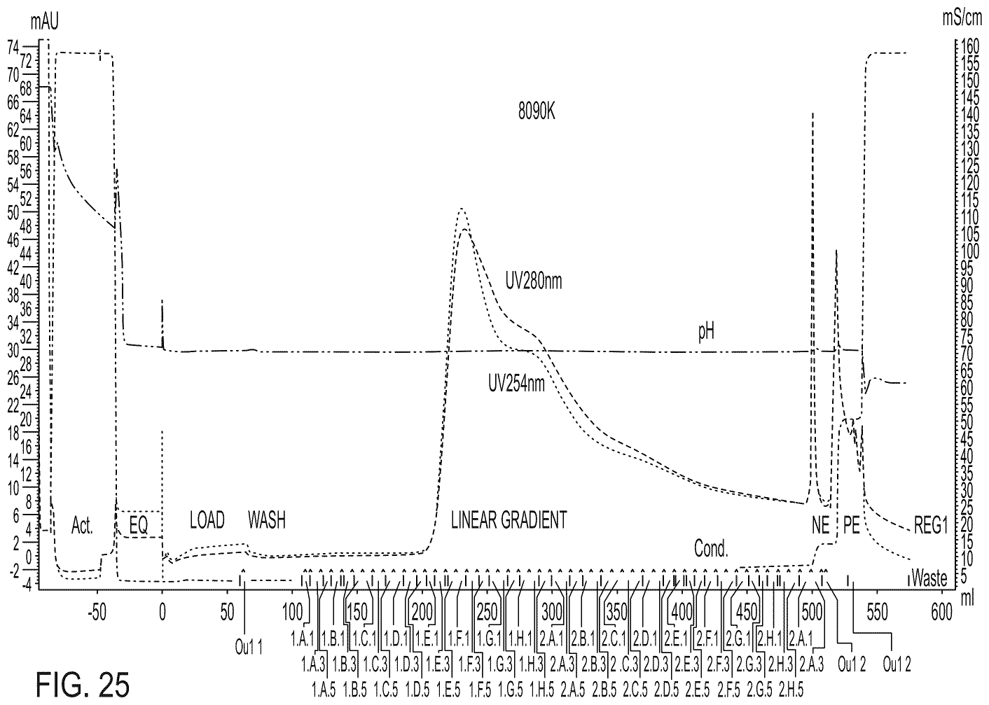

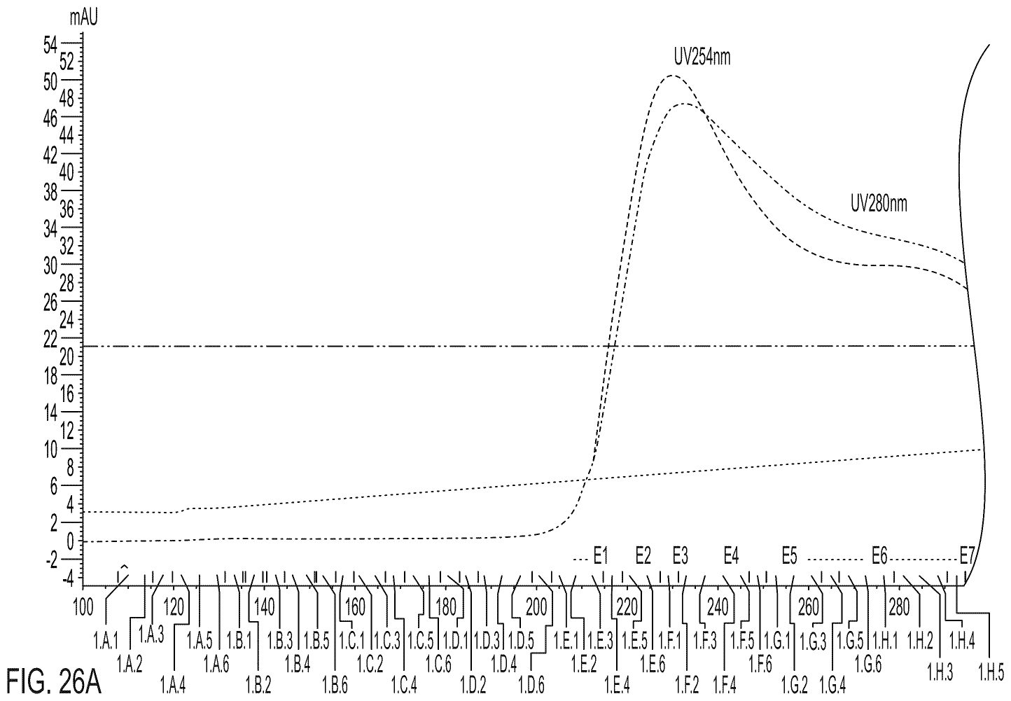

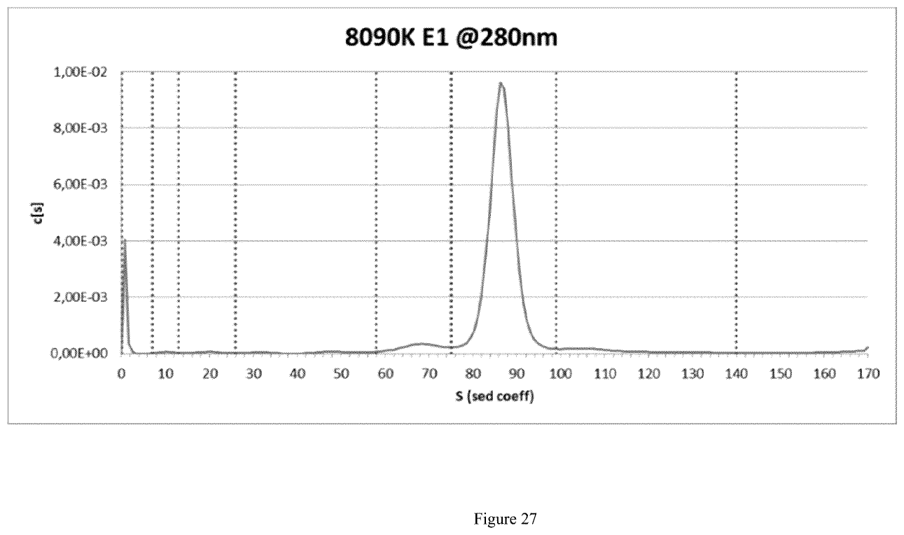

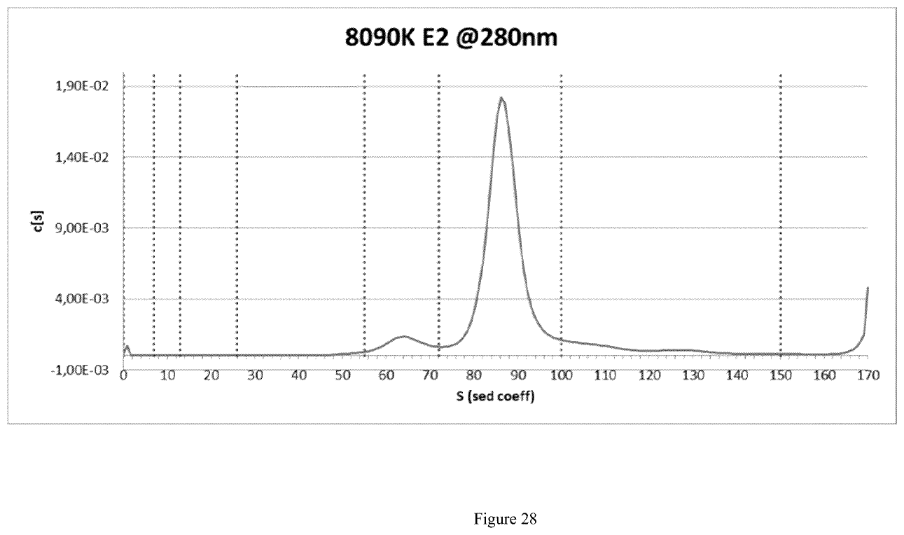

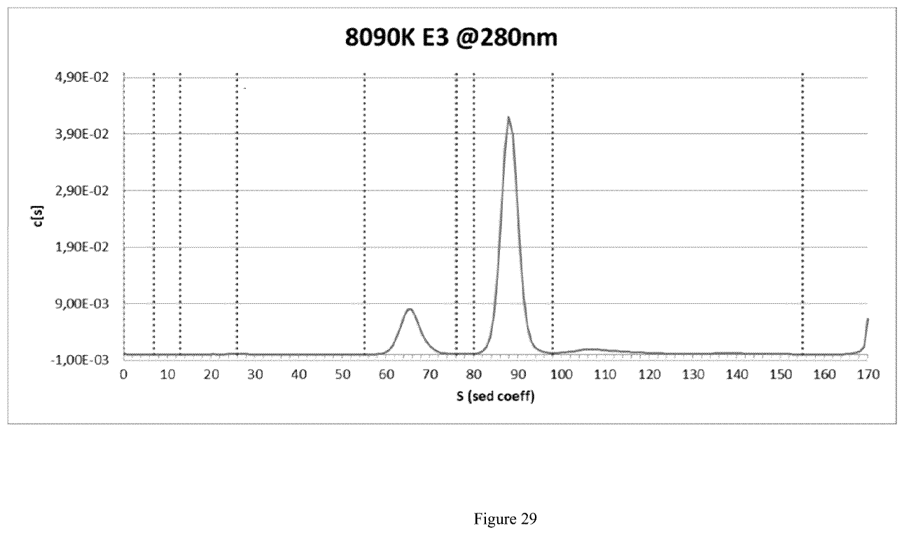

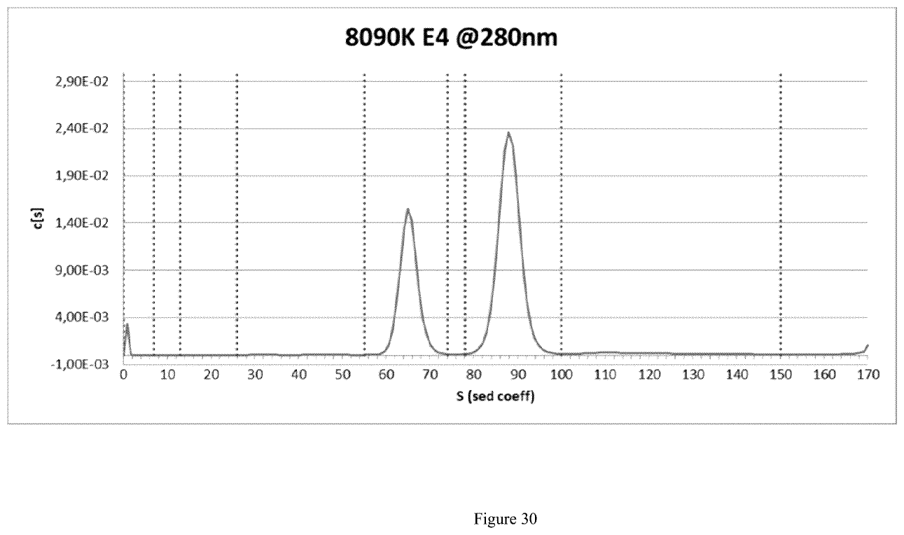

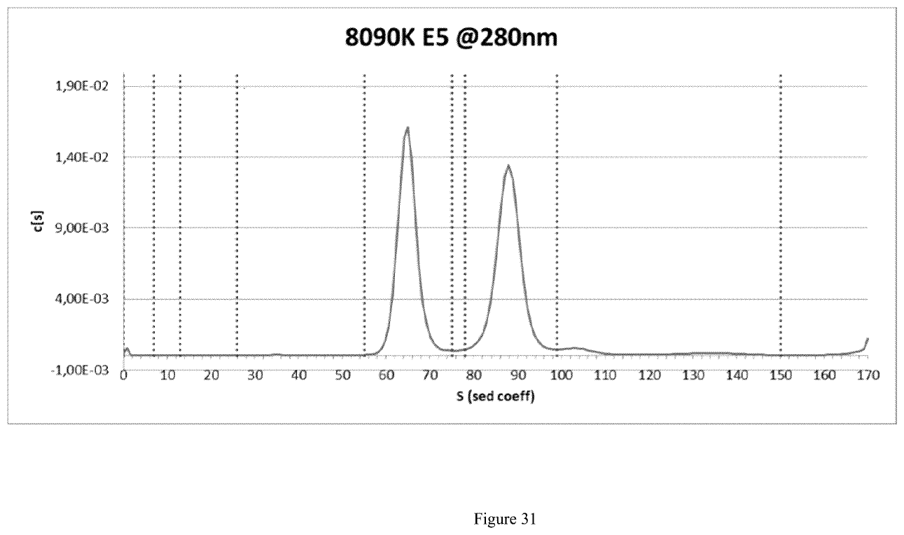

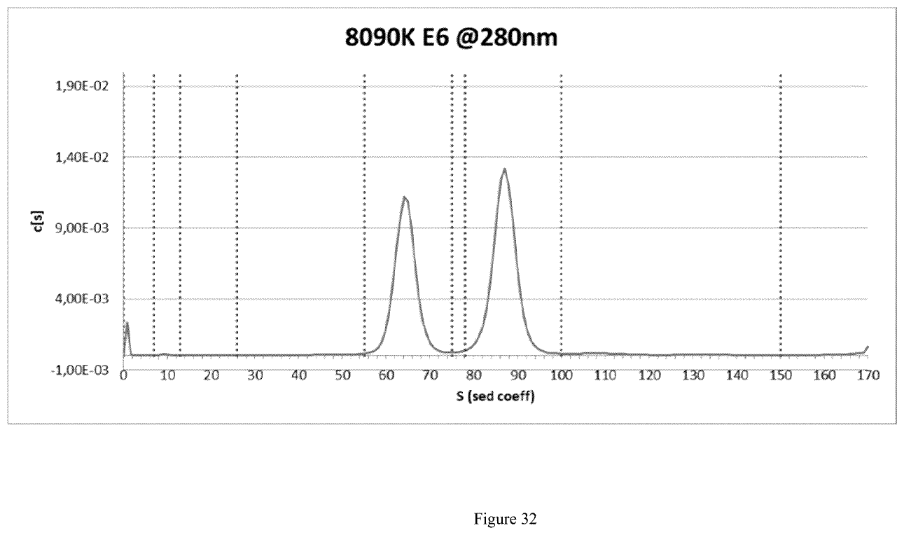

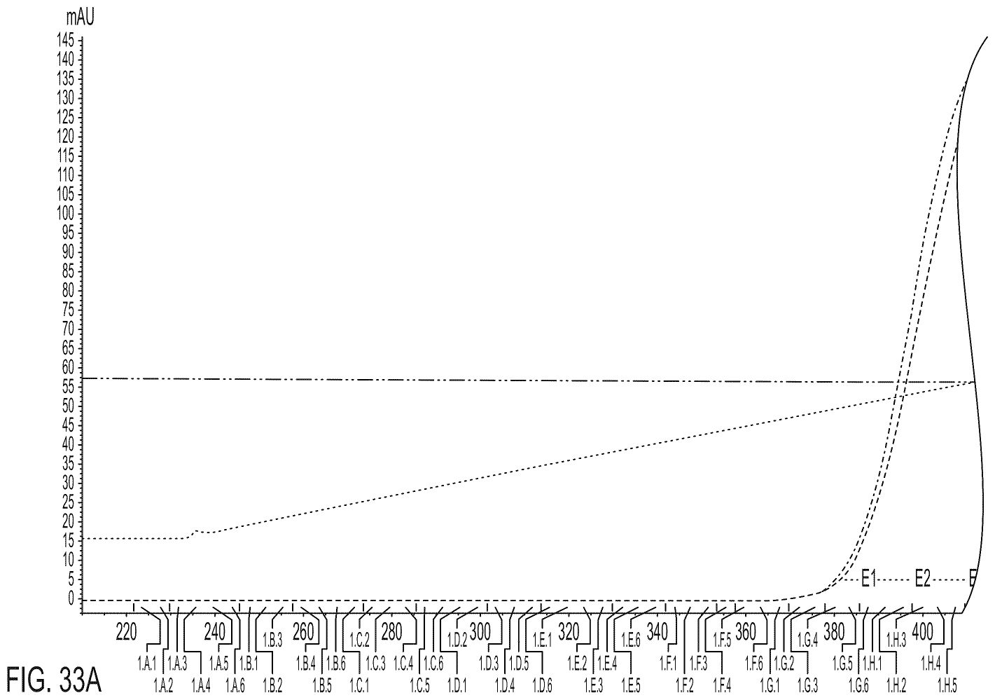

Results are shown in FIGS. 25-32 and yields are shown in Table 26.

FIGS. 25-32 represents a run with data of most of the fractions Load (Starting material, FT=Flow through, W=Wash, E=Eluate, ET-E6=Fractions of the peak (Chromatogramm). The ratio vg/cp which indicates the AAV's with the highest amount of full capsids (Full capsid fractions). The higher the value the higher is the amount of full capsids.

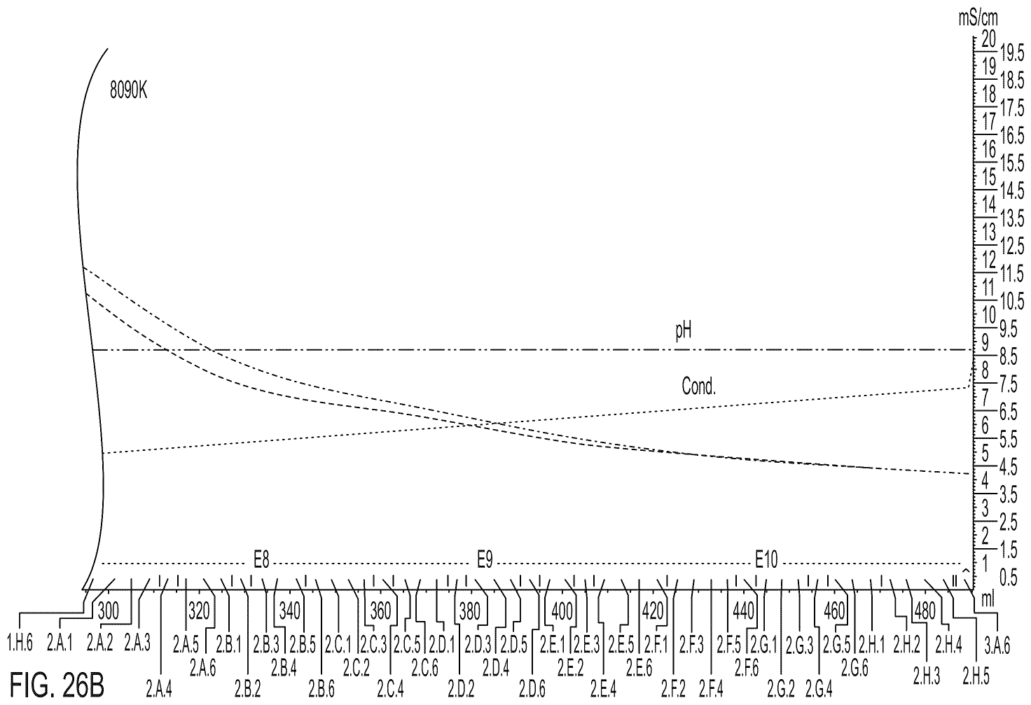

FIGS. 25 and 26 represent a chromatogram from the CEX run. It contains data of the complete run incl. regeneration procedure. Curves are shown for UV280 nm, UV254 nm, conductivity, pH, pressure, fluorescence and fractions. X-Axis: UV280 nm(left) conductivity (right), Y-Axis: Volume (ml).

TABLE 26

| | | | | | AAV8 ELISA | AAV8 ELISA | AAV8 | |
|---|---|---|---|---|---|---|---|---|
| | | FIX ddPCR | | | GTPA | GTPA | ELISA | |
| | Volume | [vg/mL] | FIX ddPCR | FIX ddPCR | [E+11 | total [E+11 | GTPA | Ratio |
| Sample code | [g] | ×10¹¹ | [vg] ×10¹¹ | Yield-[%] | cp/mL] | cp] | Yield [%] | vg/cp |
| 8093C_ | 58.73 | 80.70 | 4739.51 | 100.00% | 314.10 | 18447.09 | 100.00% | 0.26 |
| LOAD | | | | | | | | |
| 8090M_FT | 112.44 | <LOQ | — | — | <0.0319 | — | — | — |
| 8090M_W | n.a. | — | — | — | — | — | — | — |
| 8090M_E1 | 22.29 | 4.39 | 97.85 | 2.06% | 22.10 | 492.61 | 2.67% | 0.20 |
| 8090M_E2 | 7.45 | 52.20 | 388.89 | 8.21% | 31.80 | 236.91 | 1.28% | 1.64 |
| 8090M_E3 | 7.52 | 215.00 | 1616.80 | 34.11% | 124.00 | 932.48 | 5.05% | 1.73 |
| 8090M_E4 | 7.48 | 43.30 | 323.88 | 6.83% | 161.80 | 1210.26 | 6.56% | 0.27 |
| 8090M_E5 | 7.50 | 25.80 | 193.50 | 4.08% | 180.60 | 1354.50 | 7.34% | 0.14 |
| 8090M_E6 | 11.29 | 25.20 | 284.51 | 6.00% | 161.30 | 1821.08 | 9.87% | 0.16 |
| 8090M_E7 | 15.03 | 10.20 | 153.31 | 3.23% | 97.40 | 1463.92 | 7.94% | 0.10 |
| 8090M_E8 | 22.48 | 6.63 | 149.04 | 3.14% | 48.60 | 1092.53 | 5.92% | 0.14 |
| 8090M_E9 | 104.82 | 1.33 | 139.41 | 2.94% | 10.44 | 1094.32 | 5.93% | 0.13 |
| 8090M_NE | 98.35 | 2.34 | 230.14 | 4.86% | 11.60 | 1140.86 | 6.18% | 0.20 |
| 8090M_REG | 102.43 | 0.58 | 58.90 | 1.24% | 3.80 | 389.23 | 2.11% | 0.15 |
| Recovery | | | | 76.72% | | | 60.87% | |

### Example 7: AAV9, Vector Genome Size 2.6 kB, Eshmuno S, in Presence of Calcium Preparation of the Load Material

AAV9 production was developed in a HEK293 cell line after transfection with a triple plasmid system containing encoding cDNA of the protein of interest and AAV9-. VP1.-VP2 and -VP3. The clarified cell free culture supernatant was concentrated and diafiltrated with Pall Omega T-Series Cassette 100 kDa. The viral particles were loaded onto a membrane adsorber (MustangQ. Pall Part Number XT140MSTGQP05) at nonbinding conditions. The obtained AAV9 containing flow through was used as Load for the following affinity purification step.

The following test procedure was undertaken. Note that all buffers disclosed in this example were made at room temperature and the pH of all buffers were measured at room temperature. A column containing POROS™ CaptureSelect™ AAVX Affinity Matrix (Cat. No. A36742, Thermo Fisher) ID 11 mm, with a bed height of 57 mm and a volume approx. 5.4 ml, was equilibrated with at least five column volumes of 50 mM TrisHCl and 125 mM NaCl at pH 8.5. The LOAD was applied onto the column containing POROS™ CaptureSelect™ AAVX Affinity Matrix (Cat. No. A36742 Thermo Fisher). The column was then re-equilibrated with 5 column volumes of 50 mM TrisHCl and 125 mM NaCl at pH 8.5. The column was then washed with 5 column volumes of Wash 1 (W1): 100 mM Sodium Acetate and 0.1% Tween80 at pH 6.0. The column was then washed with 5 column volumes of Wash 2 (W2): 50 mM TrisHCl and 125 mM NaCl at pH 8.5. A further wash with 5 column volumes of 100 mM Sodium Acetate and 0.1% Tween80 at pH 6.0 was applied. All these steps were performed at room temperature.

Elution was undertaken lowering the temperature to +2° C. to +8° C. and flow rate to 5 cm/h by applying 10 column volumes of 100 mM Sodium Acetate and 0.1% Tween80 at pH 6.0 to the column and the procedure is shown in more detail in Table B.

TABLE B
Purification scheme for AAVX-Affinity (AAV9)
“Cold elution protocol”

| Step | High pH LOAD | CV | Temperature | Flowrate |
|---|---|---|---|---|
| 1. | 50 mM TrisHCl | ≥5 | RT | 60 cm/h |
| | 125 mM NaCl | | | |
| | pH 8.5 | | | |
| 2. | Sample-Load | | RT | |
| | pH 8.5 | | | |
| 3. | 50 mM TrisHCl | 5 | RT | |
| | 125 mM NaCl | | | |
| | pH 8.5 | | | |
| 4. | 100 mM NaAcetat | 5 | RT | |
| | 0.1% Tween80 | | | |
| | pH 6.0 | | | |
| 5. | 50 mM TrisHCl | 5 | RT | |
| | 125 mM NaCl | | | |
| | pH 8.5 | | | |
| 6. | 100 mM NaAcetat | 5 | RT | |
| | 0.1% Tween80 | | | |
| | pH 6.0 | | | |
| 7. | ELUTION | 10 | +2 to 8° C. | 5 cm/h |
| | 100 mM NaAcetat | | | |
| | 0.1% Tween80 | | | |
| | pH 6.0 | | | |

RT: Room temperature +18° C. to +26° C.

A buffer exchange of the eluate into 30 mM NaAcetate, 2 mM CaAcetate, 0.005% Polysorbate 80, pH 6.0 was performed with VIVACELL100 (10K) cartridges (Sartorius) to provide proper binding properties for all AAV-Subtypes on cation exchanger (CEX).

The following test procedure was undertaken. Note that all buffers disclosed in this example were made at room temperature and the pH of all buffers were measured at room temperature. First, a column containing Eshmuno S Cation Exchanger Resin (Cat. 1.20078; Merck-Millipore) ID 11 mm, with a bed height of 100 mm, an area of 0.95 cm², and a volume of approximately 9.5 ml, was equilibrated (activation) with 5 column volumes of a buffer comprising 1000 mM Sodium acetate, 2 mM Calcium acetate, 0.005% Polysorbate 80 at a pH of 6.0. The column was then equilibrated with at least five column volumes of 30 mM Sodium acetate, 2 mM Calcium acetate, 0.005% Polysorbate 80 at a pH of 6.0. The LOAD (AAV conditioned in 30 mM Sodium acetate, 2 mM Calcium acetate, 0.005% Polysorbate 80 at a pH of 6.0) was applied onto the column containing Eshmuno S Cation Exchanger Resin.

The column was then washed with five column volumes of 30 mM Sodium acetate, 2 mM Calcium acetate, 0.005% Polysorbate 80 at a pH of 6.0.

For the elution a Gradient elution was then performed. The Gradient was performed with 40 column volumes of 30 mM Sodium acetate, 2 mM Calcium acetate, 0.005% Polysorbate 80 at a pH of 6.0 to 200 mM Sodium acetate, 2 mM Calcium acetate, 0.005% Polysorbate 80 at a pH of 6.0. Post elution was performed with 10 column volumes of 1000 mM Sodium acetate, 2 mM Calcium acetate, 0.005% Polysorbate 80 at a pH of 6.0.

For all steps the linear flow rate was 60 cm/h.

Buffer compositions are summarized in Table 27.

TABLE 27
Buffer composition for CEX-Separation

| Buffer code | Buffer content | pH |
|---|---|---|
| TWA (2M NaCl) | 2000 mmol NaCl | n.d |
| AC1 | 30 mM NaAcetate | pH 6.0 ± 0.2 |
| | 2 mM CaAcetate | |
| | 0.005% Polysorbate 80 | |
| ACE | 200 mM NaAcetate | pH 6.0 ± 0.2 |
| | 2 mM CaAcetate | |
| | 0.005% Polysorbate 80 | |
| ACNE | 1000 mM NaAcetate | pH 6.0 ± 0.2 |
| | 2 mM CaAcetate | |
| | 0.005% Polysorbate 80 | |

Table 28 shows the chromatographic scheme for CEX-Separation

TABLE 28

| Step | | Buffer | Amount CV | Flow rate cm/h |
|---|---|---|---|---|
| 1 | Equilibration 1 | TWA (2M NaCl) | 5 | 60 |
| 2 | Equilibration 2 | AC1 | 5 | 60 |
| 3 | Product load | diluted affinity | x | 60 |
| | | Eluate | | |
| 4 | Wash 1 | AC1 | 5 | 60 |
| 5 | Elution | Gradient 0-100% | 40 | 60 |
| | | ACE in AC1 | | |
| | Collecting | | 4ml Fractions | |
| 7 | Post elution | ACNE | 10 | 60 |

Table 29 shows the ratio of ddPCR/AAV9:AG

TABLE 29

| Sample code | Ratio HT2 ddPCR/AAV9:AG vg/cp |
|---|---|
| LOAD | 0.46 |
| E1 | 0.47 |
| E2 | 0.41 |
| E3 | 0.46 |
| E4 | 0.37 |
| E5 | 0.30 |
| E6 | 0.23 |
| E7 | 0.17 |

ddPCR: HT2 ddPCR/AAV9:AG determined with AAV9 Antigen ELISA

Table 30 shows the percentage of full and empty capsids determined with AUC.

TABLE 30

| | Area % | Area % | Area % | Area % | S | S | S | S |
|---|---|---|---|---|---|---|---|---|
| Name | empty | subpopulation | Full | Aggregates | empty | subpopulation | Full | Aggregates |
| LOAD | 21.7 | 8.6 | 61.8 | 7.9 | 64.9 | 79.4 | 91.4 | 101.7 |
| E2 | 10.2 | 6.5 | 80.7 | 2.7 | 63.2 | 76.0 | 88.8 | 101.7 |
| E3 | 20.8 | 8.4 | 67.0 | 3.9 | 62.4 | 75.2 | 88.0 | 99.9 |
| E4 | 23.0 | 11.1 | 58.7 | 7.3 | 62.4 | 73.5 | 88.0 | 98.2 |
| E5 | 27.9 | 9.6 | 57.6 | 4.9 | 62.4 | 74.3 | 87.1 | 98.2 |

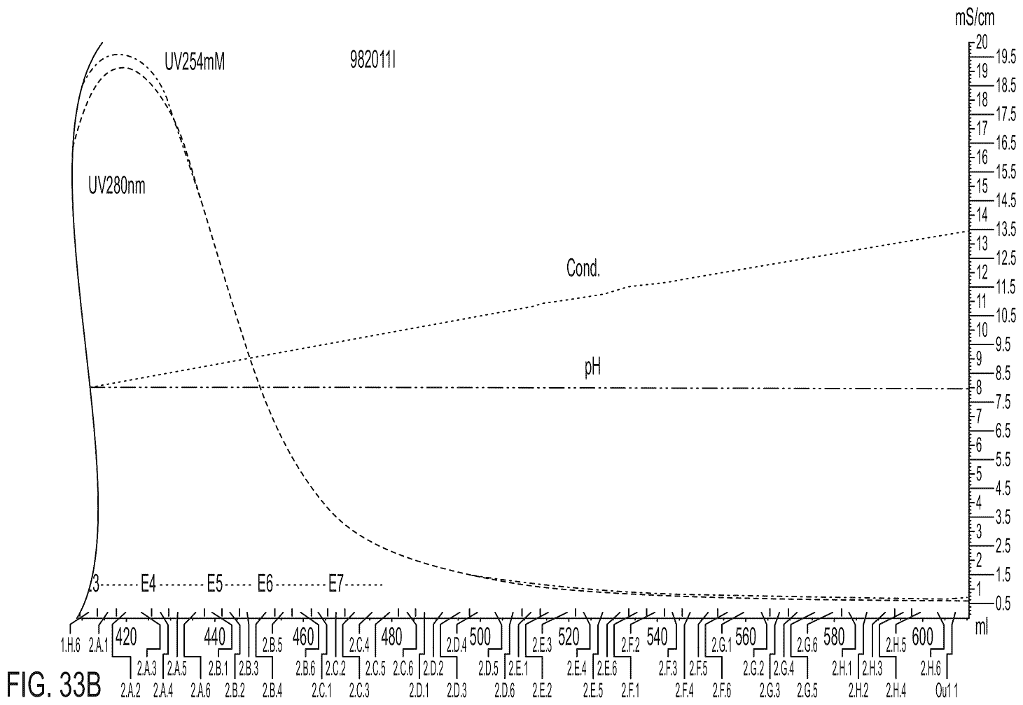

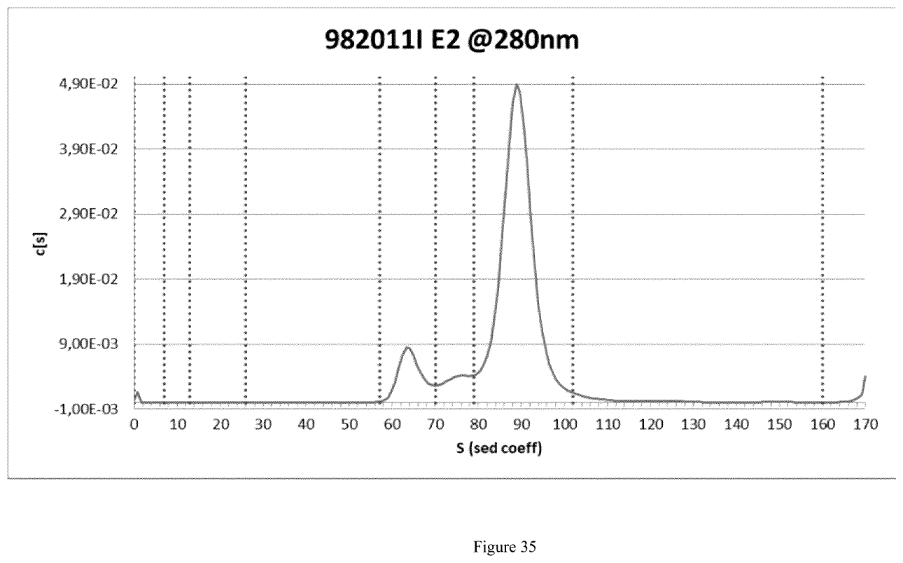

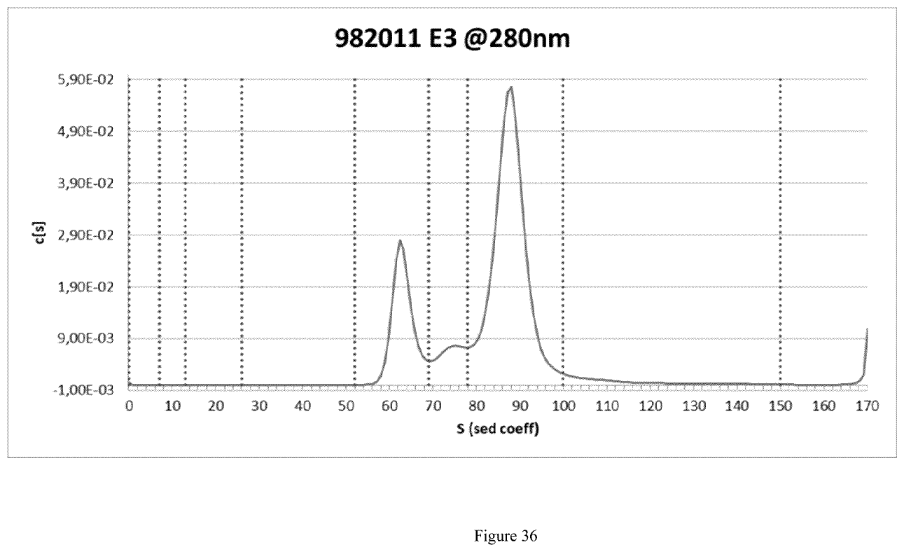

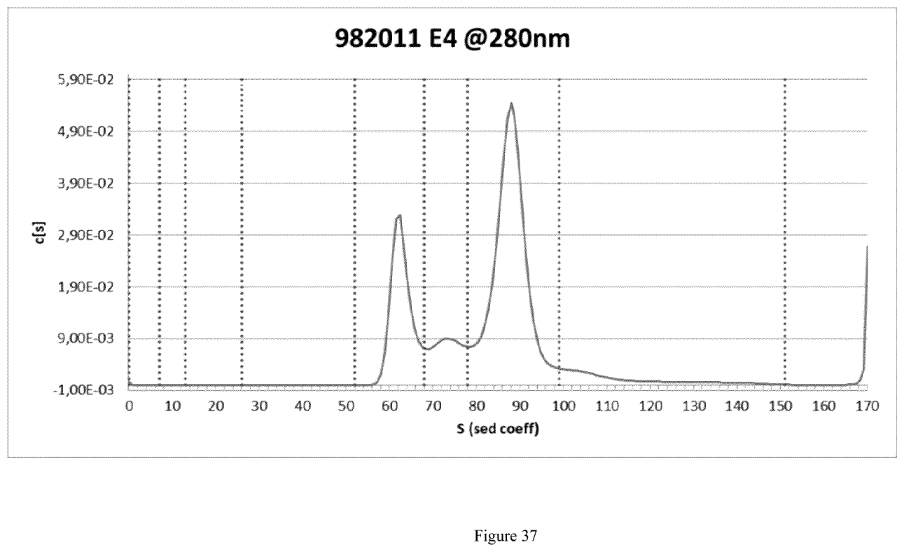

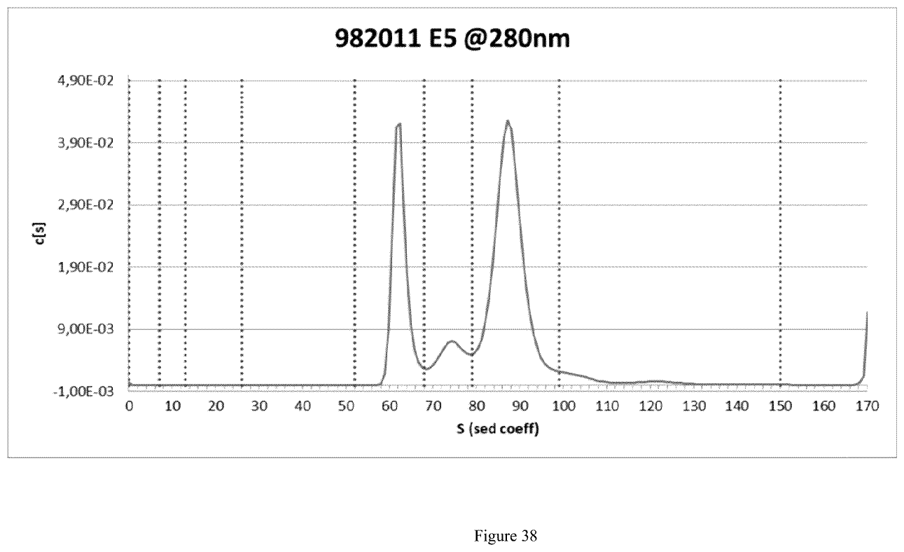

Results are shown in FIGS. 33-38 and yields are shown in Table 31.

FIGS. 33-38 represents a run with data of most of the fractions Load (Starting material, FT=Flow through, W=Wash, E=Eluate, ET-E6=Fractions of the peak (Chromatogramm). The ratio vg/cp which indicates the AAV's with the highest amount of full capsids (Full capsid fractions). The higher the value the higher is the amount of full capsids.

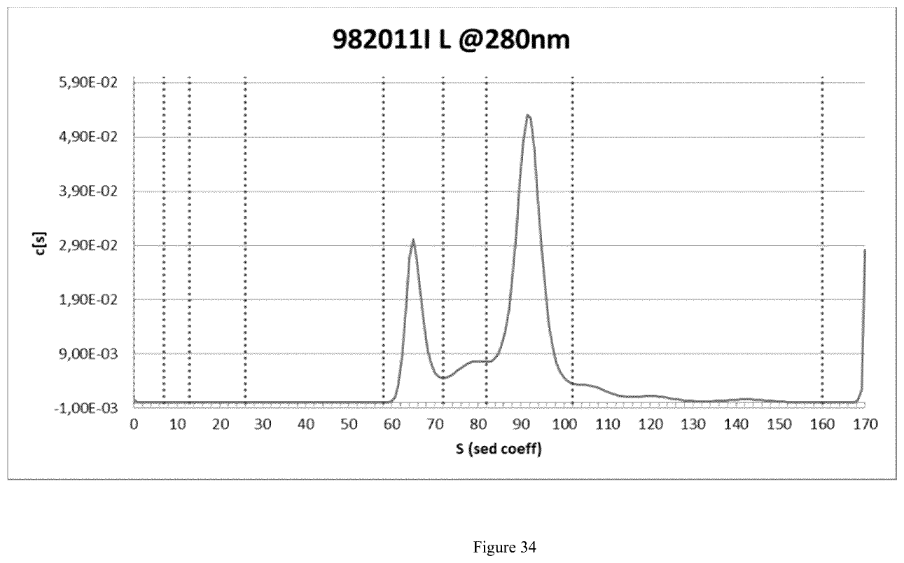

FIGS. 33 and 34 represent a chromatogram from the CEX run. It contains data of the complete run incl. regeneration procedure. Curves are shown for UV280 nm, UV254 nm, conductivity, pH, pressure, fluorescence and fractions. X-Axis: UV280 nm(left) conductivity (right), Y-Axis: Volume (ml).

TABLE 31

| | | HT2 | | | | | | |
|---|---|---|---|---|---|---|---|---|
| | | ddPCR | | | | AAV9 ELISA | | |
| | Volumen | [vg/mL] | HT2 ddPCR | HT2 ddPCR | AAV9 ELISA | total | AAV9 ELISA | Ratio |
| Sample code | [g] | ×10¹¹ | [vg] ×10¹¹ | Yield [%] | [cp/mL] ×10¹¹ | [cp] ×10¹¹ | Yield [%] | vg/cp |
| 98209H_LOAD | 92.88 | 38.7 | 3594.46 | 100.00% | 81.30 | 7551.14 | 100.00% | 0.48 |
| 98209H_FT | 143.16 | <LOQ | — | — | <0.0383 | — | — | — |
| 98209H_E1 | 19.53 | 35.8 | 699.17 | 19.45% | 81.17 | 1585.25 | 20.99% | 0.44 |
| 98209H_E2 | 7.77 | 82.8 | 643.36 | 17.90% | 227.5 | 1767.29 | 23.40% | 0.36 |
| 98209H_E3 | 15.57 | 44.7 | 695.98 | 19.36% | 139.2 | 2166.72 | 28.69% | 0.32 |
| 98209H_E4 | 31.20 | 7.28 | 227.14 | 6.32% | 26.3 | 819.31 | 10.85% | 0.28 |
| 98209H_E5 | 81.86 | 0.96 | 78.18 | 2.17% | 3.3 | 269.73 | 3.57% | 0.29 |
| 98209H_E6 | 7.73 | 11.6 | 89.67 | 2.49% | 29.9 | 230.82 | 3.06% | 0.39 |
| 98209H_NE | 49.50 | 0.24 | 11.73 | 0.33% | 0.8 | 38.07 | 0.50% | 0.31 |
| 98209H_Strip | 50.36 | 0.16 | 7.81 | 0.22% | 0.6 | 28.86 | 0.38% | 0.27 |
| Recovery | | | | 68.24% | | | 91.46% | |

1. A method for purifying full AAV capsids from an AAV preparation comprising full AAV capsids and empty AAV capsids, to provide an AAV product substantially free of empty AAV capsids comprising the steps of:
(a) providing a first solution comprising full AAV capsids, empty AAV capsids, one or more monovalent cations, and one or more divalent cations;
(b) loading said first solution onto a cation exchange column under conditions whereby said full AAV capsids and said empty AAV capsids bind to the column; and
(c) adding a second solution comprising one or more monovalent cations and one or more divalent cations to the cation exchange column under conditions wherein the full AAV capsids are purified from the empty AAV capsids.

2. A method of separating full AAV capsids and empty AAV capsids in an AAV preparation comprising the steps of:
(a) providing a first solution comprising full AAV capsids, empty AAV capsids, one or more monovalent cations, and one or more divalent cations;
(b) loading said first solution onto a cation exchange column under conditions whereby said full AAV capsids and said empty AAV capsids bind to the column; and
(c) adding a second solution comprising one or more monovalent cations and one or more divalent cations to the cation exchange column under conditions wherein the full AAV capsids are separated from the empty AAV capsids.

3. The method of claim 1 or claim 2, wherein the one or more monovalent cations of the first solution is selected from the group consisting of Na⁺, K⁺, NH₄⁺, Li⁺, Cs⁺, and combinations thereof.

4. The method of any one of claims 1-3, wherein the one or more monovalent cation of the first solution is Na⁺.

5. The method of any of claims 1-4, wherein the one or more monovalent cations of the first solution is in a total concentration of about 5 mM to about 1500 mM.

6. The method of claim 5, wherein the one or more monovalent cations of the first solution is in a total concentration of about 30 mM.

7. The method of any one of claims 1-6, wherein the one or more divalent cations of the first solution is selected from the group consisting of Ca²⁺, Mg²⁺, Zn²⁺, Mn²⁺, Cu²⁺, Fe²⁺, Ba²⁺, Sr²⁺, Co²⁺, and combinations thereof.

8. The method of claim 7, wherein the one or more divalent cation of the first solution is Ca²⁺.

9. The method of any one of claims 1-8, wherein the one or more divalent cations of the first solution is in a total concentration of about 1 mM to about 30 mM.

10. The method of claim 9, wherein the one or more divalent cations of the first solution is in a total concentration of about 2 mM.

11. The method of any one of claims 1-10, wherein the first solution has a pH of about 5.0 to about 8.5.

12. The method of claim 11, wherein the first solution has a pH of about 6.0.

13. The method of any one of claims 1-12, where in the first solution further comprises one or more surfactants.

14. The method of claim 13, wherein the one or more surfactants is selected from the group consisting of polysorbate 20, polysorbate 40, polysorbate 65, polysorbate 80, polyoxyethylene glycol tert-octylphenol ether, sorbitan monolaurate, sorbitan monopalmitate, sorbitan monostearate, sorbitan tristearate, sorbitan monooleate, sorbitan trioleate, polyoxyethylene (20) sorbitan monopalmitate, polyoxyethylene (20) sorbitan monostearate, polyoxyethylene (20) sorbitan tristearate, polyoxyethylene (20) sorbitan trioleate, polyoxyethylen(20)-sorbitan-monooleate (Tween 80/Polysorbate 80)), poloxamer 124, poloxamer 188, poloxamer 407, cremophor, Triton N-101 reduced, Triton X-100, and combinations thereof.

15. The method of claim 14, wherein the surfactant is polysorbate 80.

16. The method of any one of claims 13-15, wherein the one or more surfactants is in a total amount of about 0.0025 w/w % to about 0.0075 w/w %.

17. The method of claim 16, wherein the one or more surfactants is in a total amount of about 0.005 w/w %.

18. The method of any one of claims 1-17, wherein the cation exchange column comprises a resin with a charged group wherein the charged group is sulfonate, sulfate, sulfopropyl, carboxyl, phosphate, or combinations thereof.

19. The method of any one of claims 1-18Error! Reference source not found., wherein the cation exchange column comprises a resin wherein it the resin is CaptoS, Eshmuno S, Mustang S, Poros 50HS, Poros 50 XS, S-Sepharose FF, Source S, Capto MMC, Toyopearl Gigacap S, Gigacap CM, Toyopearl SP, Toyopearl CM, MacroPrep S, UNOsphereS, MacroprepCM, Fractogel EMD S03, Fractogel EMD COO, Fractogel EMD SE Hicap, Cellufine Sulfate, CM and SP Trisacryl, CM and S HyperD, S and CM Sepharose CL, CM Sepharose FF, S and CM CAPTO™, MonoS, Nuvia S, Cellufine phosphat, Cellufine MAX-S r, Cellufine MAX-S h, Cellufine MAX DexS-HbP, Cellufine MAX DexS-VirS, Toyopearl Sulfate 650, or Heparin Sepharose High Performance.

20. The method of claim 19, wherein the resin is CaptoS.

21. The method of claim 19, wherein the resin is Eshmuno S.

22. The method of claim 19, wherein the resin is Mustang S.

23. The method of any one of claims 1-22, wherein the one or more monovalent cations of the second solution is selected from the group consisting of Na⁺, K⁺, NH₄⁺, Li⁺, Cs⁺, and combinations thereof.

24. The method of claim 23, wherein the monovalent cation of the second solution is Na⁺.

25. The method of any one of claims 1-24, wherein the one or more divalent cations of the second solution is selected from the group consisting of Ca²⁺, Mg²⁺, Zn²⁺, Mn²⁺, Cu²⁺, Fe²⁺, Ba²⁺, Sr²⁺, and combinations thereof.

26. The method of claim 25, wherein the one or more divalent cation of the second solution is Ca²⁺.

27. The method of any one of claims 1-27, wherein the second solution has a pH of about 5.0 to about 8.5.

28. The method of claim 27, wherein the second solution has a pH of about 6.0.

29. The method of any one of claims 1-28, where in the second solution further comprises one or more surfactants.

30. The method of claim 29, wherein the one or more surfactants is selected from the group consisting of polysorbate 20, polysorbate 40, polysorbate 65, polysorbate 80, polyoxyethylene glycol tert-octylphenol ether, sorbitan monolaurate, sorbitan monopalmitate, sorbitan monostearate, sorbitan tristearate, sorbitan monooleate, sorbitan trioleate, polyoxyethylene (20) sorbitan monopalmitate, polyoxyethylene (20) sorbitan monostearate, polyoxyethylene (20) sorbitan tristearate, polyoxyethylene (20) sorbitan trioleate, polyoxyethylen(20)-sorbitan-monooleate (Tween 80/Polysorbate 80)), poloxamer 124, poloxamer 188, poloxamer 407, cremophor, Triton N-101 reduced, Triton X-100, and combinations thereof.

31. The method of claim 30, wherein the surfactant is polysorbate 80.

32. The method of any one of claims 29-31, wherein the one or more surfactants is in a total amount of about 0.00 w/w25% to about 0.0075 w/w %.

33. The method of claim 32, wherein the one or more surfactants is in a total amount of about 0.005 w/w %.

34. The method of any one of claims 1-33, wherein the adding the second solution is carried out at a constant total concentration of the one or more monovalent cations.

35. The method of claim 34, wherein the one or more monovalent cations of the second solution is in a constant total concentration of about 5 mM to about 1500 mM.

36. The method of claim 35, wherein the one or more monovalent cations of the second solution is in a constant total concentration of about 30 mM.

37. The method of any one of claims 1-33, wherein the adding the second solution is carried out at a constant total concentration of the one or more divalent cations.

38. The method of any one of claim 37, wherein the one or more divalent cations of the second solution is in a constant total concentration of about 1 mM to about 30 mM.

39. The method of claim 38, wherein the one or more divalent cations of the second solution is in a constant total concentration of about 2 mM.

40. The method of any one of claims 1-33, wherein the adding the second solution comprises a stepwise increase of the total concentration of the one or more monovalent cations.

41. The method of claim 40, wherein the initial total concentration of the one or more monovalent cations of the second solution is about 15 mM to about 60 mM.

42. The method of claim 41, wherein the initial total concentration of the one or more monovalent cations of the second solution is about 30 mM.

43. The method of any one of claims 40-42, wherein the intermediate total concentration of the one or more monovalent cations of the second solution is about 100 mM to about 300 mM.

44. The method of claim 43, wherein the intermediate total concentration of the one or more monovalent cations of the second solution is about 200 mM.

45. The method of any one of claims 40-44, wherein the final total concentration of the one or more monovalent cations of the second solution is about 500 mM to about 1500 mM.

46. The method of claim 45, wherein the final total concentration of the one or more monovalent cations of the second solution is about 1000 mM.

47. The method of any one of claims 1-33, wherein the adding the second solution comprises a stepwise increase of the total concentration of the one or more divalent cations.

48. The method of claim 47, wherein the initial total concentration of the one or more divalent cations of the second solution is about 1 mM to about 10 mM.

49. The method of claim 47 or claim 48, wherein the intermediate total concentration of the one or more divalent cations of the second solution is about 10 mM to about 20 mM.

50. The method of any one of claims 47-49, wherein the final total concentration of the one or more divalent cations of the second solution is about 20 mM to about 30 mM.

51. The method of any one of claims 1-33, wherein the adding the second solution comprises a continuous linear increase of the total concentration of the one or more monovalent cations.

52. The method of claim 51, wherein the continuous linear increase of the total concentration of the one or more monovalent cations of the second solution is about 30 mM to about 200 mM in 40 column volumes.

53. The method of claim 52, wherein the continuous linear increase of the total concentration of the one or more monovalent cations of the second solution is about 30 mM to about 80 mM in 40 column volumes.

54. The method of claim 52, wherein the continuous linear increase of the total concentration of the one or more monovalent cations of the second solution is about 30 mM to about 40 mM in 40 column volumes.

55. The method of claim 51, wherein the continuous linear increase of the total concentration of the one or more monovalent cations of the second solution is about 30 mM to about 200 mM in 5 column volumes.

56. The method of claim 55, wherein the continuous linear increase of the total concentration of the one or more monovalent cations of the second solution is about 30 mM to about 80 mM in 5 column volumes.

57. The method of claim 55, wherein the continuous linear increase of the total concentration of the one or more monovalent cations of the second solution is about 30 mM to about 80 mM in 5 column volumes.

58. The method of any one of claims 1-33 and 51-54, wherein the adding the second solution comprises a continuous linear increase of the total concentration of the one or more divalent cations.

59. The method of claim 58, wherein the continuous linear increase of the total concentration of the one or more divalent cations of the second solution is about 1 mM to about 30 mM in 40 column volumes.

60. The method of claim 58, wherein the continuous linear increase of the total concentration of the one or more divalent cations of the second solution is about 1 mM to about 15 mM in 40 column volumes.

61. The method of claim 58, wherein the continuous linear increase of the total concentration of the one or more divalent cations of the second solution is about 1 mM to about 5 mM in 40 column volumes.

62. The method of claim 58, wherein the continuous linear increase of the total concentration of the one or more divalent cations of the second solution is about 1 mM to about 30 mM in 5 column volumes.

63. The method of claim 62, wherein the continuous linear increase of the total concentration of the one or more divalent cations of the second solution is about 1 mM to about 15 mM in 5 column volumes.

64. The method of claim 62, wherein the continuous linear increase of the total concentration of the one or more divalent cations of the second solution is about 1 mM to about 5 mM in 5 column volumes.

65. The method of any one of the preceding claims, wherein the AAV capsid is derived from the group consisting of AAV2, AAV3, AAV3b, AAV4, AAV5, AAV6, AAV7, AAV8, AAV9, AAV10, AAV11, AAV12, genetical modified AAV, chemical modified AAV, genetical and chemical modified AAV, and combinations thereof.

66. The method of claim 65, wherein the AAV capsid is derived from AAV8.

67. The method of claim 65, wherein the AAV capsid is derived from AAV9.

68. The method of claim 65, wherein the AAV capsid is derived from AAV6.

69. An AAV formulation comprising full AAV capsids purified according to the method of any one of claims 1-68 in a pharmaceutically acceptable carrier, wherein the AAV formulation is substantially free of empty AAV capsids.

70. A pharmaceutical composition comprising an AAV product produced by a method according to any one of claims 1-68.

71. A method for purifying empty AAV capsids from an AAV preparation comprising empty AAV capsids and full AAV capsids, to provide an AAV product substantially free of empty AAV capsids comprising the steps of:
(a) providing a first solution comprising empty AAV capsids, full AAV capsids, one or more monovalent cations, and one or more divalent cations;
(b) loading said first solution onto a cation exchange column under conditions whereby said empty AAV capsids and said full AAV capsids bind to the column; and
(c) adding a second solution comprising one or more monovalent cations and one or more divalent cations to the cation exchange column under conditions wherein the empty AAV capsids are purified from the full AAV capsids.

72. A method of separating empty AAV capsids and full AAV capsids in an AAV preparation comprising the steps of:
(a) providing a first solution comprising empty AAV capsids, full AAV capsids, one or more monovalent cations, and one or more divalent cations;
(b) loading said first solution onto a cation exchange column under conditions whereby said empty AAV capsids and said full AAV capsids bind to the column; and
(c) adding a second solution comprising one or more monovalent cations and one or more divalent cations to the cation exchange column under conditions wherein the empty AAV capsids are separated from the full AAV capsids.

73. The method of claim 71 or 72, wherein the empty AAV capsid is derived from the group consisting of AAV2, AAV3, AAV3b, AAV4, AAV5, AAV6, AAV7, AAV8, AAV9, AAV10, AAV11, AAV12, genetical modified AAV, chemical modified AAV, genetical and chemical modified AAV, and combinations thereof.

74. The method of claim 73, wherein the empty AAV capsid is derived from AAV8.

75. The method of claim 73, wherein the empty AAV capsid is derived from AAV9.

76. The method of claim 73, wherein the empty AAV capsid is derived from AAV6.

77. A method of preparing an immune absorption column comprising the steps of
(a) concentrating the empty AAV capsids of claim 71 or 72 by ultrafiltration;
(b) applying a buffer exchange into an amine free buffer; and
(c) immobilizing the empty AAV capsids on an activated resin.

78. A method of preparing an immune absorption column comprising the steps of
(a) concentrating the empty AAV capsids of claim 71 or 72 by anion exchanger;
(b) applying a buffer exchange into an amine free buffer; and
(c) immobilizing the empty AAV capsids on an activated resin.

79. A method of preparing an immune absorption column comprising the steps of
(a) concentrating the empty AAV capsids of claim 71 or 72 a cation exchanger;
(b) applying a buffer exchange into an amine free buffer; and
(c) immobilizing the empty AAV capsids on an activated resin.

80. A method of preparing an immune absorption column comprising the steps of
(a) concentrating the full AAV capsids of claim 1 or 2 by ultrafiltration; and
(b) applying a buffer exchange into a buffer suitable for treating a patient.

81. A method of preparing an immune absorption column comprising the steps of
(a) concentrating the full AAV capsids of claim 1 or 2 by anion exchanger; and
(b) applying a buffer exchange into a buffer suitable for treating a patient.

82. A method of preparing an immune absorption column comprising the steps of
(a) concentrating the full AAV capsids of claim 1 or 2 a cation exchanger; and
(b) applying a buffer exchange into a buffer suitable for treating a patient.

83. The methods of any one of claims 1-68, wherein the AAV product comprises less than about 30% of empty AAV capsids.

84. The methods of claim 83, wherein the AAV product comprises less than about 20% of empty AAV capsids.

85. The methods of claim 84, wherein the AAV product comprises less than about 6% of empty AAV capsids.

86. The AAV formulation of claim 69 or the composition of claim 70, wherein the AAV formulation or composition comprises less than about 30% of empty AAV capsids.

87. The AAV formulation or the composition of claim 87, wherein the AAV formulation or composition comprises less than about 20% of empty AAV capsids.

88. The AAV formulation or the composition of claim 87, wherein the AAV formulation or composition comprises less than about 6% of empty AAV capsids.
